(19) United States
(12) Patent Application Publication
(10) Pub. No.: US 2022/0127644 A1
(43) Pub. Date: Apr. 28, 2022

(54) CHIMERIC ANTIGEN RECEPTOR (CAR) NK CELLS AND USES THEREOF

(71) Applicant: RESEARCH INSTITUTE AT NATIONWIDE CHILDREN'S HOSPITAL, Columbus, OH (US)

(72) Inventors: Meisam Naeimi Kararoudi, Columbus, OH (US); Dean Anthony Lee, Canal Winchester, OH (US)

(21) Appl. No.: 17/511,187

(22) Filed: Oct. 26, 2021

Related U.S. Application Data
(60) Provisional application No. 63/105,722, filed on Oct. 26, 2020.

(51) Int. Cl. C12N 15/90; A61K 35/17; C07K 14/725; C07K 16/28; C07K 14/73; C07K 14/705; C12N 5/0783; A61P 35/00

(52) U.S. Cl. CPC C12N 15/907; C12N 2310/20; C07K 14/7051; C07K 16/2803; C07K 14/70514; C07K 14/70517; C07K 14/70521; C07K 14/70578; C07K 14/7056; C12N 5/0646; A61P 35/00; C07K 2319/03; C07K 2319/02; C07K 2317/622; C12N 2501/2302; A61K 35/17

(57) ABSTRACT

Disclosed are plasmid and methods for genetically engineering NK cells using Adeno-associated viral (AAV) delivery of a CRISPR/CAS9 system. In some aspects, disclosed herein are method of using such engineering NK cells for treating cancers.

### I. CROSS-REFERENCE TO RELATED APPLICATIONS

This application claims the benefit of U.S. Provisional Application No. 63/105,722, filed Oct. 26, 2020, which is expressly incorporated herein by reference in its entirety.

### II. BACKGROUND

Human peripheral blood natural killer (NK) cells have intense antitumor activity and have been used successfully in several clinical trials. Modifying NK cells with a chimeric antigen receptor (CAR) can improve their targeting and increase specificity. However, genetic modification of NK cells has been challenging due to the high expression of innate sensing mechanisms for viral nucleic acids. What are needed are new methods and vectors for engineering NK cells.

### III. SUMMARY

Disclosed are methods and compositions related to electroporation of NK cells for delivery of a CRISPR/CAS9 gene editing system to a cell (e.g., NK cell).

In one aspect, disclosed herein are plasmids for use with clustered regularly interspaced short palindromic repeat (CRISPR)/CRISPR-associated 9 (Cas9) integration systems wherein the plasmid comprises in order a left homology arm, a polynucleotide sequence encoding a chimeric antigen receptor (CAR) polypeptide (such as, for example, a CAR comprising a scFv targeted to a receptor on a target cell (e.g., CD33), a transmembrane domain (e.g., an NKG2D transmembrane domain, a CD4 transmembrane domain, a CD8 transmembrane domain, a CD28 transmembrane domain, and/or a CD3ξ transmembrane domain), a costimulatory domain (e.g., a 2B4 domain, a CD28 co-stimulatory domain, a 4-1 BB co-stimulatory domain, or any combination of a 2B4 domain, a CD28 co-stimulatory domain, and/or a 4-1 BB co-stimulatory domain), and a CD3ξ signaling domain), and a right homology arm; wherein the left and right homology arms are each 1000 bp in length or less (for example, 30 bp in length, 300 bp in length, 600 bp in length).

Also disclosed herein are plasmids for use with CRISPR/Cas9 integration systems of any preceding aspect, wherein the left homology arm and right homology arm are the same length or different lengths. In some aspects, the homology arms specifically hybridize to the Adeno-Associated Virus Integration Site 1 (AAVS1) of chromosome 19 of humans.

In some embodiments, disclosed herein are plasmids for use with CRISPR/Cas9 integration systems of any preceding aspect, wherein the plasmid further comprises a murine leukemia virus-derived (MND) promoter.

Also disclosed herein are Adeno-associated viral (AAV) vectors (such as, for example, an AAV vector comprising the AAV6 serotype) comprising the plasmid of any preceding aspect. In some aspects, AAV plasmids further comprise a polynucleotide sequence encoding a chimeric antigen receptor (CAR) polypeptide. In some embodiments, the vector further comprises a plasmid encoding a crRNA, a tracer RNA (trcrRNA), and a Cas endonuclease. The AAV vector can be a single stranded AAV (ssAAV) or a self-complimentary AAV (scAAV).

In one aspect, disclosed herein are modified cells (such as, for example NK cells and NK T cells) comprising the plasmid or the AAV vector of any preceding aspect.

Also disclosed herein are methods of treating, decreasing, reducing, inhibiting, ameliorating, and/or preventing a cancer and/or metastasis (such as, for example, acute lymphocytic leukemia (ALL), acute myeloid leukemia (AML), chronic myeloid leukemia (CML), hairy cell leukemia (HCL), and/or myelodysplastic syndromes (MDS)) in a subject comprising administering to a subject with a cancer the modified cell of any preceding aspect.

In one aspect, disclosed herein are methods creating a chimeric antigen receptor (CAR) natural killer (NK) cell or CAR NK T cell comprising a) obtaining a ribonucleoprotein (RNP) complex comprising a class 2 CRISPR/Cas endonuclease (Cas9) complexed with a corresponding CRISPR/Cas guide RNA and an AAV vector comprising a plasmid comprising a transgene (such as, for example, a chimeric antigen receptor for a tumor antigen); wherein the transgene is flanked by homology arms; and wherein the homology arms are 1000 bp in length or less; and b) introducing the transgene and the RNP complex into an NK cell or NK T cell; wherein the transgene (such as, for example, a chimeric antigen receptor for a tumor antigen) is introduced into the NK cell or NK T cell via infection with the Adeno-associated virus (AAV); wherein the RNP complex hybridizes to a target sequence within the genomic DNA of the NK cell or NK T cell and the DNA repair enzymes of the NK cell or NK T cell insert the transgene into the host genome (for example, by homologous repair) at the target sequence, thereby creating a CAR NK cell or CAR NK T cell. In some aspects, the RNP complex can be introduced into the cell via electroporation. In some aspects, the RNP complex can be introduced into the cell via viral delivery in the same or a different AAV (i.e., superinfection).

In one aspect, disclosed herein are methods of genetically modifying a cell (T cells, B cells, macrophages, NK cells, NK T cells, fibroblasts, osteoblasts, hepatocytes, neuronal cells, epithelial cells, and/or muscle cells, including, but not limited to primary or expanded cells) comprising a) obtaining a ribonucleoprotein (RNP) complex comprising a class 2 CRISPR/Cas endonuclease (Cas9) complexed with a corresponding CRISPR/Cas guide RNA and an AAV vector comprising a plasmid comprising a chimeric antigen receptor (CAR) polypeptide; wherein the polynucleotide sequence is flanked by homology arms; and wherein the homology arms are 1000 bp in length or less; and b) introducing the polynucleotide sequence and the RNP complex into the cell; wherein the polynucleotide sequence is introduced into the cell via infection with the AAV into the cell; wherein the RNP complex hybridizes to a target sequence within the genomic DNA of the cell and the cell's DNA repair enzymes insert the transgene into the host genome at the target sequence within the genomic DNA of the cell thereby creating a modified cell.

In some embodiments, disclosed herein are methods of genetically modifying a cell of any preceding aspect, wherein the cell (e.g., NK cell or NK T cell) is infected with about 5 to 500K multiplicity of infection (MOI) of the AAV disclosed herein.

Also disclosed herein are methods of genetically modifying a cell of any preceding aspect, wherein the primary cells are incubated for about 4 to 10 days in the presence of IL-2 and/or irradiated feeder, plasma membrane particles, or exosomes cells prior to infection and/or electroporation. In some embodiments, disclosed herein are methods of genetically modifying a cell of any preceding aspect further comprising expanding the primary cells for about 4 to 10 days in the presence of irradiated feeder cells, plasma membrane particles, or exosomes prior to infection, wherein the irradiated feeder cells, plasma membrane particles, or exosomes express membrane bound 4-1BBL, membrane-bound IL-21, or membrane-bound IL-15, or any combination thereof. Also disclosed herein are methods of genetically modifying a cell of any preceding aspect, further comprising expanding the modified cell with irradiated feeder cells, plasma membrane particles, or exosomes following infection, wherein the irradiated feeder cells, plasma membrane particles, or exosomes express membrane bound 4-1BBL, membrane-bound IL-21, or membrane-bound IL-15, or any combination thereof.

In some aspects, disclosed herein is a method of treating, decreasing, reducing, inhibiting, ameliorating, and/or preventing a cancer and/or metastasis (such as, for example, acute lymphocytic leukemia (ALL), acute myeloid leukemia (AML), chronic myeloid leukemia (CML), hairy cell leukemia (HCL), and/or myelodysplastic syndromes (MDS)) in a subject comprising administering to the subject a therapeutically effective amount of a natural killer (NK) cell, wherein the NK cell comprises a plasmid for use with clustered regularly interspaced short palindromic repeat (CRISPR)/CRISPR-associated 9 (Cas9) integration systems wherein the plasmid comprises in order a left homology arm, a polynucleotide sequence encoding a chimeric antigen receptor (CAR) polypeptide (such as, for example a CD33 targeting CAR), and a right homology arm; wherein the left and right homology arms are each 1000 bp in length or less (for example, 600 bp).

In some aspects, disclosed herein is a plasmid for use with clustered regularly interspaced short palindromic repeat (CRISPR)/CRISPR-associated 9 (Cas9) integration systems wherein the plasmid comprises a polynucleotide sequence encoding a chimeric antigen receptor (CAR) polypeptide; wherein the polynucleotide sequence is adjacent to one protospacer adjacent motif (PAM) and one sequence encoding crispr RNA (crRNA) or flanked by two PAMs and sequences encoding crRNAs. It some aspects, the disclosed plasmid can be used in any of the methods of treating, decreasing, reducing, inhibiting, ameliorating, and/or preventing a cancer and/or metastasis of any preceding aspect; methods of creating a CAR NK cell and/or CAR NK T cell of any preceding aspect; and/or genetically modifying a cell of any preceding aspect.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A-1E show efficient CRISPR targeting of AAVS1 in mbIL-21 expanded human primary NK cells. FIG. 1A shows schematic of steps for isolation and ex vivo expansion of NK cells using mbIL21-K562. FIG. 1B shows relative gene expression level of HR-related genes (FIG. 1C) and NHEJ-related genes in different NK cells, ***P<0.001 for all comparisons. FIG. 1D shows ATAC-seq data showing that AAVS1 has a similar chromatin accessibility between freshly isolated (Naïve), mbIL-21 expanded NK cells (n=2). FIG. 1E shows efficiency of Cas9/RNP-mediated targeting of AAVS1 in NK cells. The sequences in FIG. 1E include: SEQ ID NO: 52, SEQ ID NO: 53, SEQ ID NO: 54, SEQ ID NO: 55, SEQ ID NO: 56, SEQ ID NO: 57, SEQ ID NO: 58.

FIGS. 2A-2C show constructs of mCherry encoding DNA for insertion into AAVS1 through HR and CRISPaint. In FIG. 2A, the left panel shows that Cas9/RNP introduces DSB in AAVS1, DNA encoding gene of interest can be integrated into NK cells through HR with optimal length of Has; the right panel shows schematics of constructs design for integration of DNA encoding mCherry with HAs between 30-1000 bp for Cas9 targeting site in AAVS1 and cloned in ssAAV6 and/or scAAV6 backbone. In FIG. 2B, the top panel shows schematics of how CRISPaint gene insertion works through homology independent DNA repair pathway; the bottom panel shows schematic of construct design for insertion of DNA encoding mCherry through CRISPaint and cloned in scAAV. FIG. 2C shows schematics of workflow to electroporate Cas9/RNP and transduce day seven mbIL21 expanded IL2-stimulated NK transduced with AAV6 for gene delivery.

FIGS. 3A-3C show targeting AAVS1 in expanded CD3negativeCD56positive NK cells does not alter normal function of the cells. FIG. 3A shows representative flow cytometry analysis showing the purity of CD3negativeCD56positive NK cells isolated from healthy donor buffy coats. FIG. 3B shows schematic of workflow for electroporation of Cas9/RNP into day 7 expanded human primary NK cells to target AAVS1. FIG. 3C shows cytotoxicity assay of AAVS1KO NK cells that does not show any suppression in their antitumor activity against AML cell lines.

FIGS. 4A-4C show that combinations of AAV6 and Cas9/RNP results in efficient generation of mCherry expressing NK cells. FIG. 4A shows representative flow cytometry of human primary NK cells expressing mCherry, 2 days after CRISPR electroporation and AAV6 transduction (MOI=3×105). FIG. 4B shows efficiency of Cas9/RNP and AAV6-mediated mCherry expression in human primary NK cells through HR and CRISPaint (n=3). FIG. 4C shows stable mCherry expression in NK cells after enrichment and expansion using mbIL21 K562.

FIG. 5 shows representative flow cytometry analysis of mCherry expression level in freshly isolated NK cells electroporated with Cas9/RNP and transduced with AAV6.

FIGS. 6A-6F show successful generation of CD33CAR expressing NK cells using combination of Cas9/RNP and AAV6. FIGS. 6A and 6B show schematic of anti-CD33 CAR constructs (Gen2 and Gen4v2) with HAs for AAVS1 targeting site and cloned in ssAAV. FIG. 6C shows representative flow cytometry showing expression of CD33CAR on NK cells, 7 days after Cas9/RNP electroporation and AAV6 transduction (MOI=3×10⁵). FIG. 6D shows that MFI of CD33CAR expression of Gen2 was significantly higher than Gen4v2, **P=0.0014. FIG. 6E shows CD33CAR expression level on NK cells seven and fourteen days after transduction and electroporation showed no significant reduction (n=3). FIG. 6F shows fold expansion of CD33CAR expressing NK cells on feeder cells for 14 days starting from 3×10⁵ cells (n=3) was similar to wildtype NK cells.

FIGS. 7A-7B show representative flow cytometry analysis of CD33CAR-Gen2 expression level in day 14 NK cells before freezing and after thaw showed no reduction. FIG. 7A also shows that the freeze and thaw did not affect the enhanced cytotoxic effect of CD33CAR-Gen2 NK cells against Kasumi-1.

FIGS. 8A-8I show that CD33CAR NK cells have enhanced anti-AML activity. CD33CAR NK cells degranulate significantly higher than wildtype NK cells when cocultured with Kasumi-1, **adjusted P value=0.004. FIG. 8A and FIG. 8B HL60, *adjusted P value=0.01. FIG. 8B shows that expressing CD33CAR on NK cells also enhances antitumor activity of NK cells against Kasumi-1 as shown in representative cytotoxicity assay performed in different effector:target ratios and in three donors, ****adjusted P value<0.0001. FIG. 8C shows that this enhanced cytotoxic activity was observed against HL-60 only in CD33CAR-Gen2 NK cells (FIGS. 8E and 8F), *adjusted P value=0.01. CD33CAR-Gen2 and Gen4v2 significantly killed higher AML-10 primary cells, ****adjusted P value<0.0001 (FIGS. 8G and 8H). The improved killing was not seen against K562, **adjusted P value=0.001.

FIGS. 9A-9D show that integration of the transgene in AAVS1 locus was confirmed by PCR and TLA. FIG. 9A shows schematic of PCR primers designed inside and outside of CD33CARs encoding DNA and integrated in AAVS1. FIG. 9B shows that amplicons were amplified and visualized on 1% agar gel only in NK cells with successful CD33CAR gene insertion at AAVS1 locus (condition 1 and 2). The gene insertion in human primary NK cells also was seen when primers designed outside of the transgenes and were used to amplify AAVS1 locus in wildtype, mCherry or CD33CARs (condition 3, primers: Forward-1200 bp (2) Reverse—1200 bp (1)). FIG. 9C shows TLA sequence coverage across the human genome using designed primers to detect integration of CD33CAR-Gen2 in day 14 cells. FIG. 9D shows that the chromosomes are indicated on the y-axis, the chromosomal position on the x-axis. Identified integration site is encircled in red.

FIGS. 10A-10B shows representative flow cytometry (FIG. 10A) analysis of CD33CAR-Gen2 expression level in NK cells transduced with 10K-300K MOI of ssAAV6 encoding CD33CAR-Gen2 showed successful expression of CAR on NK cells isolated from three healthy donors (FIG. 10B).

FIG. 11 shows CD33 expression level in different cancer cells.

FIG. 12 shows representative Calcein-AM release assay of NK cells against K562.

FIGS. 13A-13B shows representative flow cytometry analysis of CD33CAR expression level 7 days (FIG. 13A) and 14 days (FIG. 13B) post electroporation and AAV6 transduction in human NK cells.

FIG. 14 shows NGS sequencing coverage (in grey) across the vector. Black arrows indicate the primer location. The blue arrows indicate the locations of the identified vector-genome breakpoint sequences (described below). The vector map is shown on the bottom. Y-axes are limited to 100×. High coverage is observed across the region between the ITR sites, vector sequence Vector: 12-4,255. Low/no coverage is observed across the Vector: 0-11 and 4,256-6, 864 indicating the backbone has not integrated in a large proportion of this sample, potentially a small subset of the sample might contain the backbone as well. Also, coverage is observed at the ITRs, indicating that next to the integration through the homology arms also ITR based integrations occurred in the sample. Sequence variants and structural variants were called in the covered regions.

FIG. 15 shows TLA sequence coverage (in grey) across the vector integration locus, human chr19:54,550,476-55,682,266. The blue arrow indicates the location of the breakpoint sequences. Y-axes are limited to 20× and 100× resp. The coverage profile this figure shows that no genomic rearrangements have occurred in the region of the integration site. From this data it is concluded that the vector has integrated as intended in human chromosome chr19: 55,115,754-55,115,767. According to the RefSeq this is in intron 1 of PPP1R12C. Other integration sites were observed between chr19: 55,115,155-55,116,371. According to the RefSeq this is also in intron 1 of PPP1R12C.

FIG. 16 shows the construct design of pAAV AAVS1(600bpHA) MND-CD33CAR(gen2) (CoOp). The sequence of the construct is SEQ ID NO: 22.

FIG. 17 shows the construct design of pAAV AAVS1(600bpHA) MND-CD33CAR(gen4v2) (CoOp). The sequence of the construct is SEQ ID NO: 23.

FIG. 18 shows kinetic assessment of cytotoxicity of non-modified (WT) and CD33-CAR-expressing expanded NK cells against K562. The assay was performed with xCelligence to monitor target viability at 15 minute intervals, using two E:T ratios. % cell lysis was calculated in reference to control wells without NK cells. Even though K562 is highly sensitive to WT expanded NK cells and serial killing is evident (>50% lysis at 0.5:1 E:T ratio), K562 does also express CD33 so the CD33 CAR enables more rapid onset of killing in both E:T ratios, and increased overall killing at the lower E:T ratio.

FIG. 19 shows kinetic assessment of cytotoxicity against Kasumi. The assay was performed as in the previous figure. In contrast to K562, Kasumi is very resistant to WT expanded NK cells, but the addition of CD33 CAR targeting to the NK cells enables more rapid onset of high-level killing with faster kinetics and increased overall killing at the both E:T ratios.

FIG. 20 shows that AML cell co-culture with WT-NK or CD33 CAR-NK cells induces AML cell death as shown in SPADE plots (colored for pRb expression indicative of viable cycling cells), green arrows indicate live AML cells while red arrows indicate dead/dying AML cells. CD33 CAR-NK cells demonstrate increased AML cell killing, surviving AML cells have reduced CD33 surface expression and increased CD38 expression, suggesting that a combination of CD33 CAR and CD38 antibody could be synergistic. This assay used a patient-derived AML cell line as the target.

FIG. 21 shows the construct design of PAMgRNA mCherry. The sequence of the construct is SEQ ID NO: 51.

FIG. 22 shows the construct design of PAMgPAMg mCherry. The sequence of the construct is SEQ ID NO: 50.

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Biocompatible” generally refers to a material and any metabolites or degradation products thereof that are generally non-toxic to the recipient and do not cause significant adverse effects to the subject.

A “control” is an alternative subject or sample used in an experiment for comparison purposes. A control can be “positive” or “negative.”

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

A DNA sequence that “encodes” a particular RNA is a DNA nucleic acid sequence that is transcribed into RNA. A DNA polynucleotide may encode an RNA (mRNA) that is translated into protein (and therefore the DNA and the mRNA both encode the protein), or a DNA polynucleotide may encode an RNA that is not translated into protein (e.g. tRNA, rRNA, microRNA (miRNA), a “non-coding” RNA (ncRNA), a guide RNA, etc.).

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.)

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “naturally-occurring” or “unmodified” or “wild type” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is wild type (and naturally occurring).

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The term “nucleic acid” as used herein means a polymer composed of nucleotides, e.g., deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides.

“Optional” or “optionally” means that the subsequently described event or circumstance may or may not occur, and that the description includes instances where said event or circumstance occurs and instances where it does not.

As used herein, “operatively linked” can indicate that the regulatory sequences useful for expression of the coding sequences of a nucleic acid are placed in the nucleic acid molecule in the appropriate positions relative to the coding sequence so as to effect expression of the coding sequence. This same definition is sometimes applied to the arrangement of coding sequences and/or transcription control elements (e.g. promoters, enhancers, and termination elements), and/or selectable markers in an expression vector. The term “operatively linked” can also refer to the arrangement of polypeptide segments within a single polypeptide chain, where the individual polypeptide segments can be, without limitation, a protein, fragments thereof, linking peptides, and/or signal peptides. The term operatively linked can refer to direct fusion of different individual polypeptides within the single polypeptides or fragments thereof where there are no intervening amino acids between the different segments as well as when the individual polypeptides are connected to one another via one or more intervening amino acids.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically, a probe can be made from any combination of nucleotides or nucleotide derivatives or analogs available in the art.

A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g., a composition comprising an agent) refers to an amount that is effective to achieve a desired therapeutic result. In some embodiments, a desired therapeutic result is the control of cancer. In some embodiments, a desired therapeutic result is the control of metastasis. In some embodiments, a desired therapeutic result is the reduction of tumor size. In some embodiments, a desired therapeutic result is the prevention and/or treatment of relapse. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

As used herein, “transgene” refers to exogenous genetic material (e.g., one or more polynucleotides) that has been or can be artificially provided to a cell. The term can be used to refer to a “recombinant” polynucleotide encoding any of the herein disclosed polypeptides that are the subject of the present disclosure. The term “recombinant” refers to a sequence (e.g., polynucleotide or polypeptide sequence) which does not occur in the cell to be artificially provided with the sequence, or is linked to another polynucleotide in an arrangement which does not occur in the cell to be artificially provided with the sequence. It is understood that “artificial” refers to non-natural occurrence in the host cell and includes manipulation by man, machine, exogenous factors (e.g., enzymes, viruses, etc.), other non-natural manipulations, or combinations thereof. A transgene can comprise a gene operably linked to a promoter (e.g., an open reading frame), although is not limited thereto. Upon artificially providing a transgene to a cell, the transgene may integrate into the host cell chromosome, exist extrachromosomally, or exist in any combination thereof.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Plasmids and Methods of Genetically Modifying Cells

Gene modification of NK cells using viral or non-viral vectors has been challenging due to robust foreign DNA- and RNA-sensing mechanisms, which may limit the efficiency of gene delivery methods into NK cells. To overcome this limitation, a new method was developed to electroporate Cas9/ribonucleoprotein complexes (Cas9/RNP) directly into human primary NK cells. This method introduces a double-strand break (DSB) in the genome of NK cells, which results in successful gene knock-out and enhanced antitumor activity. After this initial success in gene silencing, the development of a gene insertion method was further pursued. After Cas9 introduces a DSB, two independent and innate DNA repair mechanisms can be employed to repair the break: homologous recombination (HR) or non-homologous end-joining (NHEJ). In the presence of a DNA template encoding a gene of interest, the exogenous gene can be integrated into the Cas9-targeting site using either of these repair mechanisms.

Accordingly, disclosed herein are plasmids for use with clustered regularly interspaced short palindromic repeat (CRISPR)/CRISPR-associated 9 (Cas9) integration systems wherein the plasmid comprises in order a left homology arm, a polynucleotide sequence encoding a chimeric antigen receptor (CAR) polypeptide (such as, for example, a CAR comprising a scFv targeted to a receptor on a target cell (e.g., CD33), a transmembrane domain (e.g., an NKG2D transmembrane domain, a CD4 transmembrane domain, a CD8 transmembrane domain, a CD28 transmembrane domain, or a CD3ξ transmembrane domain), a costimulatory domain (e.g., a 2B4 domain, a CD28 co-stimulatory domain, a 4-1 BB co-stimulatory domain, or any combination of a 2B4 domain, a CD28 co-stimulatory domain, and/or a 4-1 BB co-stimulatory domain), and a CD3ξ signaling domain) and a right homology arm; wherein the left and right homology arms are each 1000 bp in length or less (for example, about 30 bp in length, about 300 bp in length, or about 600 bp in length).

In general, “CRISPR system” or “CRISPR integration system” refers collectively to transcripts and other elements involved in the expression of or directing the activity of CRISPR-associated “Cas” genes. In some embodiments, one or more elements of a CRISPR system is derived from a type I, type II, or type III CRISPR system. CRISPR systems are known in the art. See, e.g., U.S. Pat. No. 8,697,359, incorporated by reference herein in its entirety.

Endonuclease/RNPs (for example, a Cas9/RNP) are comprised of three components, recombinant endonuclease protein (for example, a Cas9 endonuclease) complexed with a CRISPR loci. The endonuclease complexed to the CRISPR loci can be referred to as a CRISPR/Cas guide RNA. The CRISPR loci comprises a synthetic single-guide RNA (gRNA) comprised of a RNA that can hybridize to a target sequence complexed complementary repeat RNA (crRNA) and trans complementary repeat RNA (tracrRNA). Accordingly, the CRISPR/Cas guide RNA hybridizes to a target sequence within the genomic DNA of the cell. In some cases, the class 2 CRISPR/Cas endonuclease is a type II CRISPR/Cas endonuclease. In some cases, the class 2 CRISPR/Cas endonuclease is a Cas9 polypeptide and the corresponding CRISPR/Cas guide RNA is a Cas9 guide RNA. These Cas9/RNPs are capable of cleaving genomic targets with higher efficiency as compared to foreign DNA-dependent approaches due to their delivery as functional complexes. Additionally, rapid clearance of Cas9/RNPs from the cells can reduce the off-target effects such as induction of apoptosis.

To make the RNP complex, crRNA and tracrRNA can be mixed at a 1:1, 2:1, or 1:2 ratio of concentrations between about 50 μM and about 500 μM (for example, 50 μM, 60 μM, 70 μM, 80 μM, 90 μM, 100 μM, 125 μM, 150 μM, 175 μM, 200 μM, 225 μM, 250 μM, 275 μM, 300 μM, 325 μM, 350 μM, 375 μM, 400 μM, 425 μM, 450 μM, 475 μM, or 500 μM), preferably between 100 μM and about 300 μM, most preferably about 200 μM at 95° C. for about 5 min to form a crRNA:tracrRNA complex (i.e., the guide RNA). The crRNA:tracrRNA complex can then be mixed with between about 20 μM and about 50 μM (for example 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, or 50 μM) final dilution of a Cas endonuclease (such as, for example, Cas9).

Once bound to the target sequence in the target cell, the CRISPR loci can modify the genome by introducing into the target DNA insertion or deletion of one or more base pairs, by insertion of a heterologous DNA fragment (e.g., the donor polynucleotide), by deletion of an endogenous DNA fragment, by inversion or translocation of an endogenous DNA fragment, or a combination thereof. Thus, the disclosed methods can be used to generate knock-outs, or knock-ins when combined with DNA for homologous recombination. It is shown herein that transduction via Adeno-associated viral (AAV) of Cas9/RNPs is a relatively efficient method that overcomes previous constraints of genetic modification in cells (such as, for example, T cells, B cells, macrophages, NK cells, NK T cells, fibroblasts, osteoblasts, hepatocytes, neuronal cells, epithelial cells, and/or muscle cells).

The CRISPR/Cas9 system has recently been shown to facilitate high levels of precise genome editing using Adeno-associated viral (AAV) vectors to serve as donor template DNA during homologous recombination (HR). However, the prior use of AAV has been limited, as due to their immune function, NK cells and NK T cells are resistant to viral and bacterial vectors and the induction of NK cell/NK T cell apoptosis by said vectors. Thus, prior to the present methods CRISPR/Cas modification of NK cells or NK T cells has been unsuccessful. Moreover, the maximum AAV packaging capacity of ˜4.5 kilobases limits the donor size which includes homology arms. There are recommendations that any transcript above 100 bp and any transgene is to have homology arms that are at least 800 bp for each arm with many systems employing asymmetric arms of 800 bp and 1000 bp for a total of 1800 bp. Thus, the AAV vector cannot deliver a transgene larger than ˜2.5 kb. In one aspect, disclosed herein are AAV CRISPR/CAS9 nucleotide delivery systems comprising a donor construct plasmid with homology arms between 30 bp and 1000 bp, including, but not limited to 30 bp, 50 bp, 100 bp, 110 bp, 120 bp, 130 bp, 140 bp, 150 bp, 160 bp, 170 bp, 180 bp, 190 bp, 200 bp, 210 bp, 220 bp, 230 bp, 240 bp, 250 bp, 260 bp, 270 bp, 280 bp, 290 bp, 300 bp, 310 bp, 320 bp, 330 bp, 340 bp, 350 bp, 360 bp, 370 bp, 380 bp, 390 bp, 400 bp, 410 bp, 420 bp, 430 bp, 440 bp, 450 bp, 460 bp, 470 bp, 480 bp, 490 bp, 500 bp, 510 bp, 520 bp, 530 bp, 540 bp, 550 bp, 560 bp, 570 bp, 580 bp, 590 bp, 600 bp, 610 bp, 620 bp, 630 bp, 640 bp, 650 bp, 660 bp, 670 bp, 680 bp, 690 bp, 700 bp, 710 bp, 720 bp, 730 bp, 740 bp, 750 bp, 760 bp, 770 bp, 780 bp, 790 bp, 800 bp, 810 bp, 820 bp, 830 bp, 840 bp, 850 bp, 860 bp, 870 bp, 880 bp, 890 bp, 900 bp, 910 bp, 920 bp, 930 bp, 940 bp, 950 bp, 960 bp, 970 bp, 980 bp, 990 bp, or 1000 bp. For example, the homology arms can be symmetrical 30 bp homology arms, symmetrical 300 bp homology arms, symmetrical 500 bp homology arms, symmetrical 600 bp homology arms, symmetrical 800 bp homology arms, symmetrical 1000 bp homology arms, or asymmetrical 800 bp homology arms comprising a 800 bp left homology arm (LHA) and a 1000 bp right homology arm (RHA) for homologous recombination (HR) or no homology arms at all for non-homologous end joining using homology-independent targeted integration (HITI) plasmids. In some examples, the plasmids with or without homology arms are those disclosed in International Publication Number WO2020/198675, which is incorporated herein by reference in its entirety. In some embodiments, the plasmids have clinically approved splice acceptor (SA) (SEQ ID NO: 10) and clinically approved polyadenylation terminator (PA) (such as, for example BGH polyA terminator SEQ ID NO: 11). It is understood and herein contemplated that homology arms can be symmetrical (same length on each side) or asymmetrical (different lengths on each side) to accommodate differing transgene lengths. That is, homology arm lengths can have any combination of left homology arm (LHA) length and right homology arm (RHA) length including but not limited to LHA 30 bp (SEQ ID NO: 2) and RHA 30 bp (SEQ ID NO: 1), LHA 30 bp and RHA 100 bp, LHA 30 bp and RHA 300 bp (SEQ ID NO: 3), LHA 30 bp and RHA 500 bp (SEQ ID NO: 5), LHA 30 bp and RHA 800 bp (SEQ ID NO: 7), LHA 30 bp and RHA 1000 bp, LHA 100 bp and RHA 30 bp, LHA 100 bp and RHA 100 bp, LHA 100 bp and RHA 300 bp, LHA 100 bp and RHA 500 bp, LHA 100 bp and RHA 800 bp, LHA 100 bp and RHA 1000 bp, LHA 300 bp (SEQ ID NO: 4) and RHA 30 bp, LHA 300 bp and RHA 100 bp, LHA 300 bp and RHA 300 bp, LHA 300 bp and RHA 500 bp, LHA 300 bp and RHA 800 bp, LHA 300 bp and RHA 1000 bp, LHA 500 bp (SEQ ID NO: 6) and RHA 30 bp, LHA 500 bp and RHA 100 bp, LHA 500 bp and RHA 300 bp, LHA 500 bp and RHA 500 bp, LHA 500 bp and RHA 800 bp, LHA 500 bp and RHA 1000 bp, LHA 800 bp (SEQ ID NO: 8) and RHA 30 bp, LHA 800 bp and RHA 100 bp, LHA 800 bp and RHA 300 bp, LHA 800 bp and RHA 500 bp, LHA 800 bp and RHA 800 bp, LHA 800 bp and RHA 1000 bp, LHA 1000 bp and RHA 30 bp, LHA 1000 bp and RHA 100 bp, LHA 1000 bp and RHA 300 bp, LHA 1000 bp and RHA 500 bp, LHA 1000 bp and RHA 800 bp, and LHA 1000 bp and RHA 1000 bp.

There are several ways to provide the DNA template, including viral and non-viral methods. In non-viral approaches, the single-stranded or double-stranded DNA template is typically electroporated along with Cas9/RNP, however, it has a lower efficiency in comparison to viral transduction. For viral gene delivery, adeno-associated viruses (AAV), including AAV6, were used safely in clinical trials and are useful as vectors for sensitive primary immune cells, including T-cells.

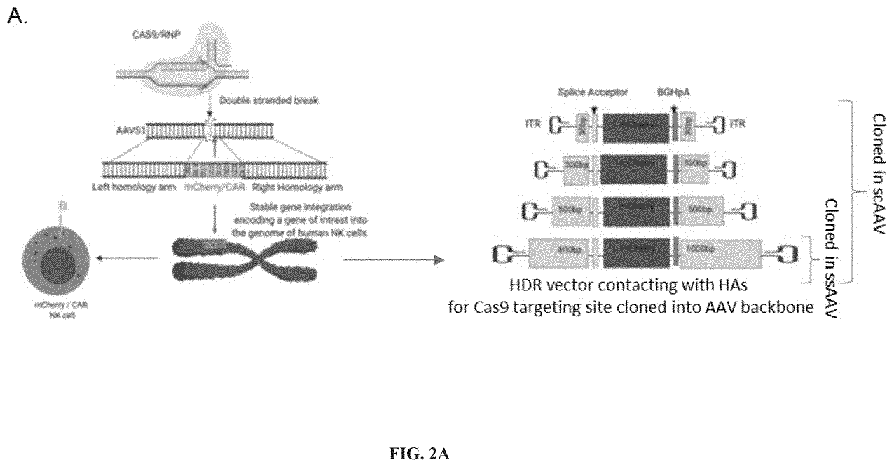

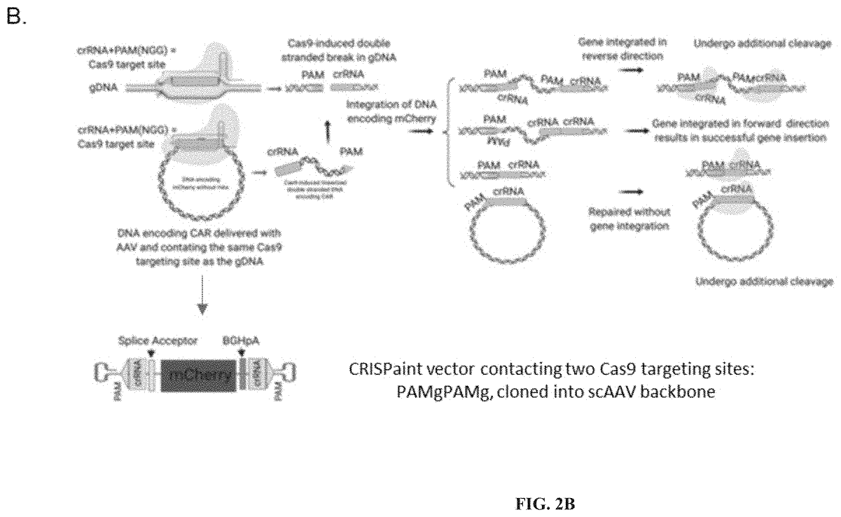

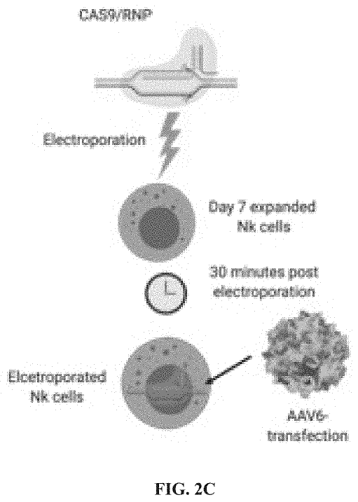

Transcripts that are delivered via AAV vectors can be packaged as a linear single-stranded (ss) DNA with a length of approximately 4.7 kb (ssAAV) or as linear self-complementary (sc) DNA (scAAV). The benefit of the scAAV vector is that it contains a mutated inverted terminal repeat (ITR), which is required for replication and helps to bypass rate-limiting steps of second strand generation in comparison to ssDNA vectors. Due to the limitation in the packaging capacity of scAAV, 30 bp, 300 bp, 500 bp, and 800-1000 bps of HAs for the right and left side of the Cas9-targeting site were designed to find the most optimal length of HAs and to provide possible lengths of HAs to be chosen based on the size of transgenes by researchers (for examples, as shown in FIG. 2A). Additionally, due to limitations in packaging capacity compared to ssAAV, scAAV may not be suitable for larger transgenes such as chimeric antigen receptor (CAR) targeting CD33. Therefore, based on the size of transgenes, both ssAAV and scAAV were designed and tested, which provides a wide range of options for gene insertion in primary NK cells and/or NK T cells.

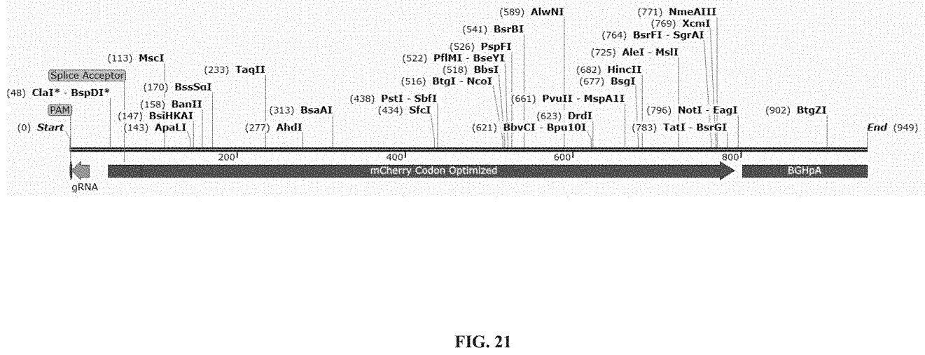

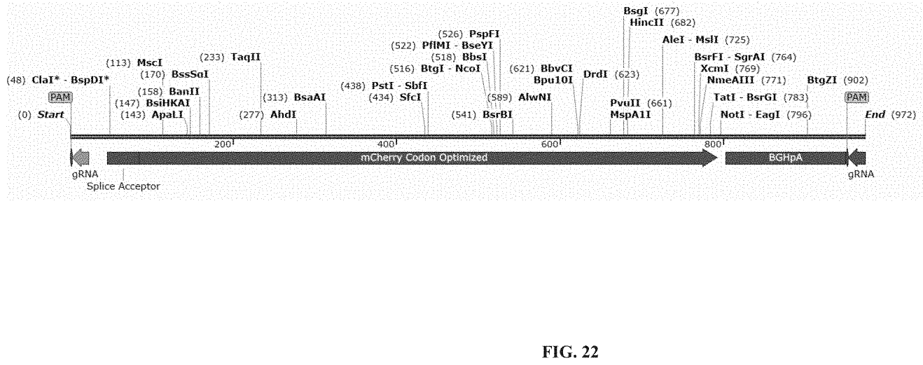

It has been shown that the efficiency of recombination increases as the length of HAs increases. Therefore, for the ssAAV backbone, the longest possible length of the left and right homology arm (HA) was used for either mCherry (e.g., 800 bp-1000 bp of HAs) and CD33 CAR-NK (e.g., 600 bp of HAs). Since designing homology arms is a time-consuming procedure and requires multiple optimizations, the CRISPaint approach has also been investigated, a homology-independent method for gene insertion or tagging. In this method, the same Cas9 targeting site, including the sequence encoding crRNA and PAM sequence (herein also termed as PAMg, e.g., SEQ ID NO: 9), is provided in the DNA template encoding the gene of interest. Upon the introduction of the Cas9 complex, both template and genomic DNA are cut simultaneously. As a result, the CRISPaint template is presented as a linearized double-stranded DNA that can be integrated through non-homology repair machinery (e.g., as shown in FIG. 2B). In some examples, the CRISPaint DNA template is as shown in FIG. 21 and FIG. 22. Accordingly, in one aspect, disclosed herein are plasmids for delivering donor transgene to a cell and integrating said transgene (e.g., CAR) into the cell in combination with CRISPR/Cas9. Thus, disclosed herein are plasmids for use with CRISPR/Cas9 integration systems of any preceding aspect, wherein the left homology arm and right homology arm are the same length or different lengths.

In some aspects, the homology arms specifically hybridize to the Adeno-Associated Virus Integration Site 1 (AAVS1) of chromosome 19 of humans. In some embodiments, the LHA is 600 bp in length. In some embodiment, the LHA comprises a sequence at least about 70% (for example, at least about 75%, 80%, 85%, 90%, 95%, 97%, or 99%) identical to SEQ ID NO: 31 or a fragment thereof. In some embodiments, the RHA is 600 bp in length. In some embodiment, the RHA comprises a sequence at least about 70% (for example, at least about 75%, 80%, 85%, 90%, 95%, 97%, or 99%) identical to SEQ ID NO: 32 or a fragment thereof.

The plasmid disclosed herein comprises a polynucleotide sequence encoding a chimeric antigen receptor CAR polypeptide. As used herein “chimeric antigen receptor” or “CAR” refers to a chimeric receptor that targets a cancer antigen and serves to bring the cell expressing the receptor to a cancer cell expressing the target antigen. Typically, the CAR comprises a molecule that recognizes peptides derived from the tumor antigen presented by major histocompatibility (MHC) molecules, or an antibody or fragment thereof (such as for example, a Fab′, scFv, Fv) expressed on the surface of the CAR cell that targets a cancer antigen. The receptor is fused to a signaling domain (such as, for example, the CD3ζ domain for T cells and NKG2C, NKp44, or CD3ζ domain for NK cells or NK T cells) via a linker. Tumor antigen targets are proteins that are produced by tumor cells that elicit an immune response, particularly B-cell, NK cell, NK T cells, and T-cell mediated immune responses. The selection of the antigen binding domain will depend on the particular type of cancer to be treated. Tumor antigens are well known in the art and include, for example, a glioma-associated antigen, carcinoembryonic antigen (CEA), EGFRvIII, IL-11Ra, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, CD123, cyclin B1, lectin-reactive AFP, Fos-related antigen 1, ADRB3, thyroglobulin, EphA2, RAGE-1, RU1, RU2, SSX2, AKAP-4, LCK, OY-TES1, PAXS, SART3, CLL-1, fucosyl GM1, GloboH, MN-CA IX, EPCAM, EVT6-AML, TGS5, human telomerase reverse transcriptase, plysialic acid, PLAC1, RU1, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6, E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRCSD, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, IL13Ra2, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG1 6, TA-90\Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

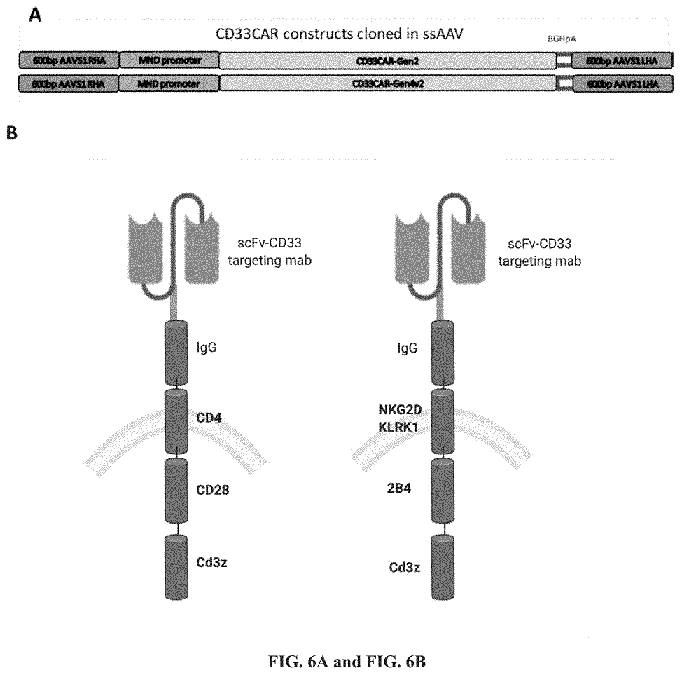

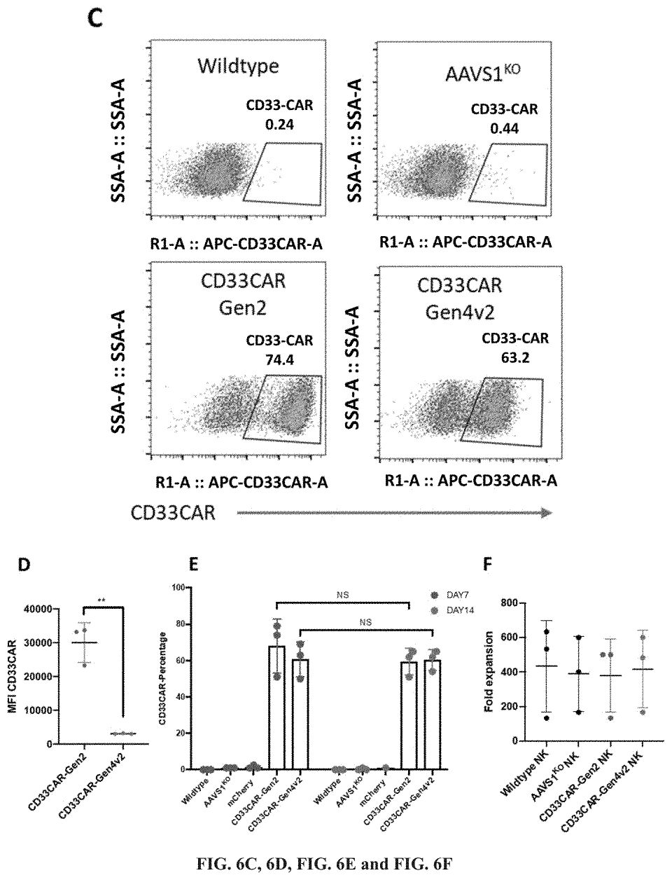

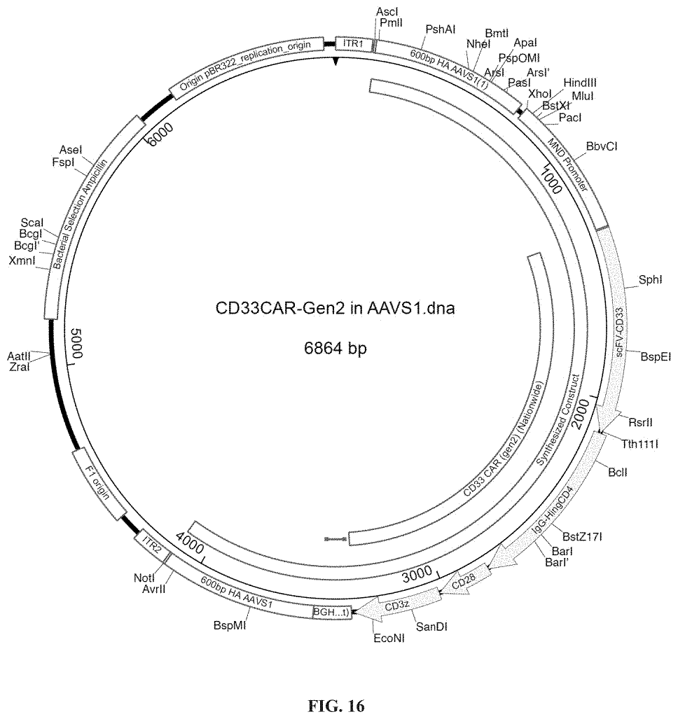

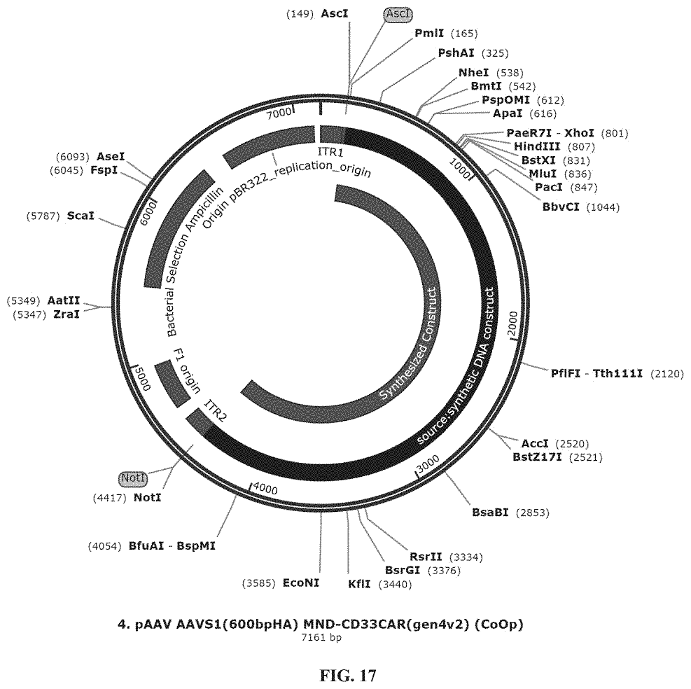

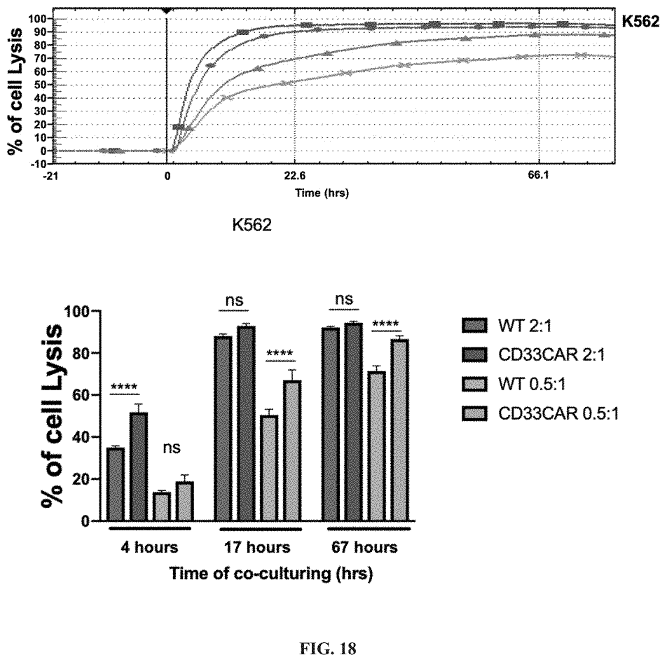

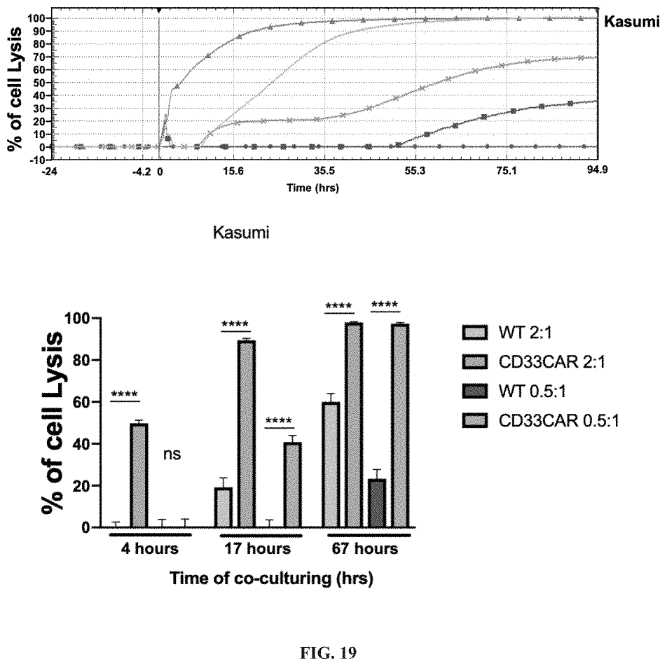

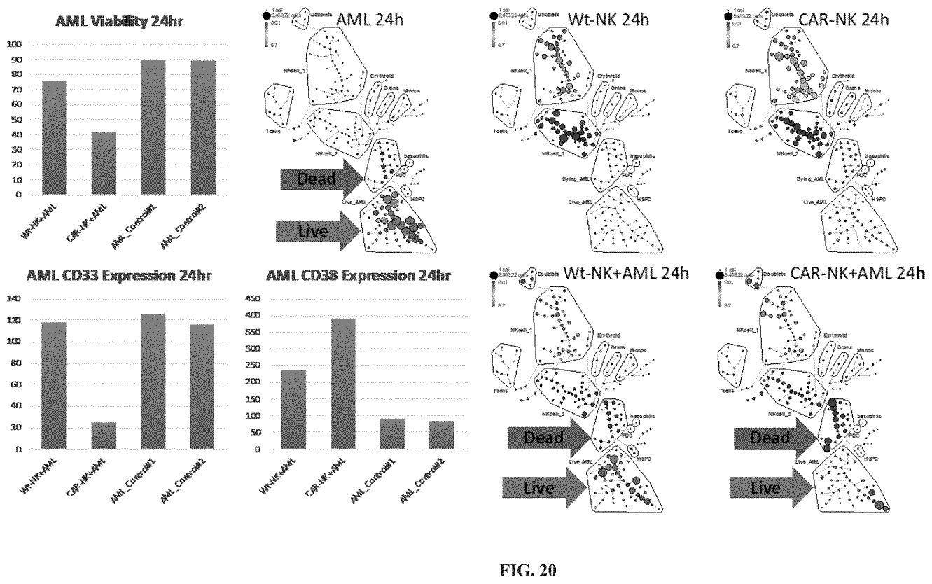

The CAR polypeptide can also comprise a transmembrane domain (such as, for example, an NKG2D transmembrane domain, a CD4 transmembrane domain, a CD8 transmembrane domain, a CD28 transmembrane domain, and/or a CD3ξ transmembrane domain) and a co-stimulatory domain (such as, for example, a 2B4 domain, a CD28 co-stimulatory domain, a 4-1 BB co-stimulatory domain, or any combination of a 2B4 domain, a CD28 co-stimulatory domain and/or a 4-1 BB co-stimulatory domain). For example, in some embodiments, the CAR polypeptide comprises a IgG4 hinge domain, a CD4 transmembrane domain, a CD28 co-stimulatory domain, a CD3zeta polypeptide, and a single-chain variable fragment (scFV) that specifically binds to a receptor on a target cell including, but not limited to, a cancer cell expressing a target antigen (for example, CD33). In some embodiments, the CAR polypeptide comprises a IgG4 hinge domain, a NKG2D transmembrane domain, a 2B4 domain, a CD3zeta polypeptide, and a single-chain variable fragment (scFV) that specifically binds to a receptor on a target cell including, but not limited to, a cancer cell expressing a target antigen (for example, CD33). In some embodiments, the CAR polypeptides are those shown in FIG. 6B. In some embodiments, the polynucleotide encoding the CAR polypeptide described herein comprises a sequence at least about 70% (for example, at least about 75%, 80%, 85%, 90%, 95%, 97%, or 99%) identical to SEQ ID NO: 22, SEQ ID NO: 23 or a fragment thereof. In some examples, the design of the plasmid comprising the CAR-coding polynucleotide is as shown in FIG. 16 and FIG. 17.

In some embodiments, the polynucleotide encoding the scFV described herein comprises a sequence at least about 70% (for example, at least about 75%, 80%, 85%, 90%, 95%, 97%, or 99%) identical to SEQ ID NO: 18 or a fragment thereof.

In some embodiments, the polynucleotide encoding the IgG4-hinge described herein comprises a sequence at least about 70% (for example, at least about 75%, 80%, 85%, 90%, 95%, 97%, or 99%) identical to SEQ ID NO: 19 or a fragment thereof.

In some embodiments, the polynucleotide encoding the CD28 co-stimulatory domain described herein comprises a sequence at least about 70% (for example, at least about 75%, 80%, 85%, 90%, 95%, 97%, or 99%) identical to SEQ ID NO: 20 or a fragment thereof.

In some embodiments, the polynucleotide encoding the CD3zeta described herein comprises a sequence at least about 70% (for example, at least about 75%, 80%, 85%, 90%, 95%, 97%, or 99%) identical to SEQ ID NO: 21, SEQ ID NO: 28, or a fragment thereof.

In some embodiments, the polynucleotide encoding the NKG2D transmembrane domain described herein comprises a sequence at least about 70% (for example, at least about 75%, 80%, 85%, 90%, 95%, 97%, or 99%) identical to SEQ ID NO: 24 or a fragment thereof.

In some embodiments, the polynucleotide encoding the 2B4 domain described herein comprises a sequence at least about 70% (for example, at least about 75%, 80%, 85%, 90%, 95%, 97%, or 99%) identical to SEQ ID NO: 26 or a fragment thereof.

In some embodiments, the polynucleotide encoding the anti-CD33 scFV comprises a sequence at least about 70% (for example, at least about 75%, 80%, 85%, 90%, 95%, 97%, or 99%) identical to SEQ ID NO: 29 or a fragment thereof.

In some embodiments, the MND promoter described herein comprises a sequence at least about 70% (for example, at least about 75%, 80%, 85%, 90%, 95%, 97%, or 99%) identical to SEQ ID NO: 30 or a fragment thereof.

In some embodiments, the expression vector described herein comprises one or more linker sequences, wherein the linker sequence comprises a sequence at least about 70% (for example, at least about 75%, 80%, 85%, 90%, 95%, 97%, or 99%) identical to SEQ ID NO: 25 or a fragment thereof.

Accordingly, in some embodiments, the plasmid disclosed herein comprises a polynucleotide sequence encoding a CAR polypeptide, wherein the CAR polypeptide comprises a transmembrane domain (e.g., an NKG2D transmembrane domain, a CD4 transmembrane domain, a CD8 transmembrane domain, a CD28 transmembrane domain, or a CD3ξ transmembrane domain), a costimulatory domain (e.g., a 2B4 domain, a CD28 co-stimulatory domain, a 4-4 BB co-stimulatory domain, or any combination of a 2B4 domain, a CD28 co-stimulator)/domain and/or a 4-1 BB co-stimulatory domain), CD3zeta, and a single-chain variable fragment (scFV) that specifically binds to a receptor on target cell (for example a cancer cell expressing CD33). In some embodiments, the CAR polypeptide specifically binds CD33.

Also disclosed herein are plasmids that can be integrated into the genome of the transduced cells via HITI, CRISPaint, or other nonhomologous end joining (NHEJ). As such, they have an advantage of integrating with higher efficiency. In some examples, the plasmids for NHEJ are those disclosed in International Publication Number WO2020/198675, which is incorporated herein by reference in its entirety. To aid in the identification of cleavage site to remove the transgene for integration, the plasmids comprise one or more PAMg sequences (i.e., the protospacer adjacent motif (PAM) and the sequence encoding crRNA (i.e., the gRNA)) (SEQ ID NO: 9) to target the donor transgene integration. In some examples, for the NHEJ DNA templates (e.g., CRISPaint DNA templates), a single (PAMg) or a double (PAMgPAMg) Cas9-targeting sequences are incorporated around the transgene (e.g., a polynucleotide encoding the CAR, such as CD33 CAR, disclosed herein) but within the ITRs. Therefore, Cas9 can simultaneously cut gDNA and the CRISPaint DNA template, enabling integration at the genomic DSB.

Accordingly, in some aspects, disclosed herein is a plasmid for use with clustered regularly interspaced short palindromic repeat (CRISPR)/CRISPR-associated 9 (Cas9) integration systems wherein the plasmid comprises a polynucleotide sequence encoding a chimeric antigen receptor (CAR) polypeptide; wherein the polynucleotide sequence is adjacent to one protospacer adjacent motif (PAM) and one polynucleotide sequence encoding crispr RNA (crRNA) or flanked by two PAMs and two polynucleotide sequences encoding crRNAs. In some aspects, disclosed herein is a plasmid for use with clustered regularly interspaced short palindromic repeat (CRISPR)/CRISPR-associated 9 (Cas9) integration systems wherein the plasmid comprises in order one protospacer adjacent motif (PAM) sequence and one polynucleotide sequence encoding crRNA, a polynucleotide sequence encoding a chimeric antigen receptor (CAR) polypeptide, and one PAM sequence and one polynucleotide sequence encoding crRNA. In some examples, the plasmid is as shown in FIGS. 2B, 21, and 22.

Additionally, despite the benefit of using the single stranded (SS) plasmids to insert the larger transgenes, SS plasmids may need more time to fold and serve as a double stranded DNA inside the cells prior to the integration which increases the DNA-sensing mechanism and cytotoxicity in some cells (such as, for example, T cells, B cells, macrophages, NK cells, fibroblasts, osteoblasts, hepatocytes, neuronal cells, epithelial cells, and/or muscle cells). This problem is overcome herein by the use of self-complementary (SC)(double stranded) constructs in order to decrease the time of exposure to the exogenous DNA in cells.

It is understood and herein contemplated that to target the Cas9 nuclease activity to the target site and also cleave the donor plasmid to allow for recombination of the donor transgene into the host DNA, a crispr RNA (crRNA) is used. In some cases, the crRNA is combined with a tracrRNA to form guide RNA (gRNA). The disclosed plasmids use AAV integration, intron 1 of the protein phosphatase 1, regulatory subunit 12C (PPP1R12C) gene on human chromosome 19, which is referred to the AAVS1, as the target site for the integration of the transgene. This locus is a “safe harbor gene” and allows stable, long-term transgene expression in many cell types. As disruption of PPP1R12C is not associated with any known disease, the AAVS1 locus is often considered a safe-harbor for transgene targeting. Because the AAVS1 site is being used as the target location, the CRSPR RNA (crRNA) must target said DNA. Herein, the guide RNA disclosed herein comprises GGGGCCACTAGGGACAGGAT (SEQ ID NO: 17) or any 10 nucleotide sense or antisense contiguous fragment thereof. Accordingly, in some examples, the PAM+the sequence encoding crRNA comprises SEQ ID NO: 9. While AAVS1 is used for exemplary purposes here, it is understood and herein contemplated that other “safe harbor genes” can be used with equivalent results and can be substituted for AAVS1 if more appropriate given the particular cell type being transfected or the transgene. Examples of other safe harbor genes, include but are not limited to C—C chemokine receptor type 5 (CCR5), the ROSA26 locus, and TRAC.

In one example, the plasmid disclosed herein further comprise a murine leukemia virus-derived (MND) promoter.

As noted above, the use of the AAV as a vector to deliver the disclosed CRISPR/Cas9 plasmid and any donor transgene is limited to a maximum of ˜4.5 kb. It is understood and herein contemplated that one method of increasing the allowable size of the transgene is to create additional room by exchanging the Cas9 of Streptococcus pyogenes (SpCas9) typically used for a synthetic Cas9, or Cas9 from a different bacterial source. Substitution of the Cas9 can also be used to increase the targeting specificity so less gRNA needs to be used. Thus, for example, the Cas9 can be derived from Staphylococcus aureus (SaCas9), Acidaminococcus sp. (AsCpf1), Lachnospiracase bacterium (LbCpf1), Neisseria meningitidis (NmCas9), Streptococcus thermophilus (StCas9), Campylobacter jejuni (CjCas9), enhanced SpCas9 (eSpCas9), SpCas9-HF1, Fokl-Fused dCas9, expanded Cas9 (xCas9), and/or catalytically dead Cas9 (dCas9).

It is understood and herein contemplated that the use of a particular Cas9 can change the PAM sequence which the Cas9 endonuclease (or alternative) uses to screen for targets. As used herein, suitable PAM sequences comprises NGG (SpCas9 PAM) NNGRRT (SaCas9 PAM) NNNNGATT (NmCAs9 PAM), NNNNRYAC (CjCas9 PAM), NNAGAAW (St), TTTV (LbCpf1 PAM and AsCpf1 PAM); TYCV (LbCpf1 PAM variant and AsCpf1 PAM variant); where N can be any nucleotide; V=A, C, or G; Y═C or T; W=A or T; and R=A or G.

In one aspect, disclosed here are methods of genetically modifying a cell comprising obtaining a ribonucleoprotein (RNP) complex comprising a class 2 CRISPR/Cas endonuclease (Cas9) complexed with a corresponding CRISPR/Cas guide RNA (gRNA) specific for a target DNA sequence in the cell and a plasmid comprising a transgene (such as, for example, a chimeric antigen receptor for a tumor antigen); wherein the transgene is flanked by homology arms; and b) introducing the transgene and the RNP complex into the cell; wherein the transgene is introduced into the cell via infection with the Adeno-associated virus (AAV) into a target cell; wherein the RNP complex hybridizes to a target sequence within the genomic DNA of the cell. In one aspect, the method can further comprise introducing the RNP complex into the cell via electroporation (such as when modifying an NK cell or NK T cell). In one aspect, the method can further comprise superinfecting the target cell with a second AAV virus comprising the RNP complex. In one aspect, where the transgene is sufficiently small, the same AAV can comprise both the transgene and the RNP complex. In still further aspects, the transgene and RNP complex can be encoded on the same plasmid.

In one aspect, disclosed herein are methods of genetically modifying a cell (e.g., an NK cell or NK T cell) comprising a) obtaining a ribonucleoprotein (RNP) complex comprising a class 2 CRISPR/Cas endonuclease (Cas9) complexed with a corresponding CRISPR/Cas guide RNA and an AAV vector comprising a plasmid comprising a transgene (such as, for example, a chimeric antigen receptor for a tumor antigen); wherein the transgene is adjacent to one PAM and crRNA or flanked by two PAMs and two sequences encoding crRNAs; and b) introducing the transgene and the RNP complex into the cell; wherein the transgene is introduced into the cell via infection with the AAV into a target cell; wherein in the ribonucleoprotein (RNP) complex hybridizes to the target sequence within the genomic DNA of the cell, and the cell's DNA repair enzymes insert the transgene into the host genome at the target sequence (for example by non-homologous end joining), thereby creating a modified cell. In one aspect, the method can further comprise introducing the RNP complex into the cell via electroporation (such as when modifying an NK cell or NK T cell). In one aspect, the method can further comprise superinfecting the target cell with a second AAV virus comprising the RNP complex. In one aspect, where the transgene is sufficiently small, the same AAV can comprise both the transgene and the RNP complex. In still further aspect, the transgene and RNP complex can be encoded on the same plasmid.

In some examples, the AAV described herein can be used as a vector to deliver the disclosed a prime-editing plasmid and any donor transgene described herein (e.g., a polynucleotide encoding CAR). Prime-editing is a “search-and-replace” genome editing technology that mediates targeted insertions, deletions base-to-base conversions, and combinations thereof in human cells without requiring DSBs or donor DNA templates. Prime-editing can uses a fusion protein that comprises a catalytically impaired Cas9 endonuclease, an engineered reverse transcriptase enzyme, an RNA-programmable nickase, and/or a prime editing guide RNA (pegRNA), to copy genetic information directly from an extension on the pegRNA into the target genomic locus. Methods for designing and using prime-editing are known in the art. See, e.g., Anzalone, A. V., Randolph, P. B., Davis, J. R. et al. Search-and-replace genome editing without double-strand breaks or donor DNA. Nature 576, 149-157 (2019), incorporated by reference herein in its entity.

It is understood and herein contemplated that the disclosed methods can be utilized with any cell type including T cells, B cells, macrophages, NK cells, NK T cells, fibroblasts, osteoblasts, hepatocytes, neuronal cells, epithelial cells, and/or muscle cells as well as any other cell type. Human NK cells are a particularly excellent target for the disclosed plasmids and methods of their use. NK cells are a subset of peripheral blood lymphocytes defined by the expression of CD56 or CD16 and the absence of T cell receptor (CD3). NK cells sense and kill target cells that lack major histocompatibility complex (MHC)-class I molecules. NK cell activating receptors include, among others, the natural cytotoxicity receptors (NKp30, NKp44 and NKp46), and lectin-like receptors NKG2D and DNAM-1. Their ligands are expressed on stressed, transformed, or infected cells but not on normal cells, making normal cells resistant to NK cell killing. NK cell activation is negatively regulated via inhibitory receptors, such as killer immunoglobin (Ig)-like receptors (KIRs), NKG2A/CD94, TGFβ, and leukocyte Ig-like receptor-1 (LIR-1). In one aspect, the target cells can be primary NK cells from a donor source, such as, for example, an allogeneic donor source for an adoptive transfer therapy or an autologous donor source (i.e., the ultimate recipient of the modified cells), NK cell line (including, but not limited to NK RPMI8866; HFWT, K562, and EBV-LCL), or from a source of expanded NK cells derived a primary NK cell source or NK cell line.

Prior to the transduction of the cells (such as, for example, T cells, B cells, macrophages, NK cells, NK T cells, fibroblasts, osteoblasts, hepatocytes, neuronal cells, epithelial cells, and/or muscle cells), the cell can be incubated in a media suitable for the propagation of the cells. It is understood and herein contemplated that the culturing conditions can comprise the addition of cytokines, antibodies, and/or feeder cells. Thus, in one aspect, disclosed herein are methods of genetically modifying a cell (such as, for example, a T cell, B cell, macrophage, NK cell, NK T cells fibroblast, osteoblast, hepatocyte, neuronal cell, epithelial cell, and/or muscle cell), further comprising incubating the cells for at least 1, 2, 3, 4, 5, 6, 7, 8 9, 10, 11, 12, 13, or 14 days prior to transducing the cells in media that supports the propagation of cells; wherein the media further comprises cytokines, antibodies, and/or feeder cells. For example, the media can comprise IL-2, IL-12, IL-15, IL-18, and/or IL-21. In one aspect, the media can also comprise anti-CD3 antibody. In one aspect, the feeder cells can be purified from feeder cells that stimulate cells. For example, NK cell stimulating feeder cells for use in the claimed invention, disclosed herein can be either irradiated autologous or allogeneic peripheral blood mononuclear cells (PBMCs) or nonirradiated autologous or PBMCs; RPMI8866; HFWT, K562; K562 cells transfected with membrane bound IL-15, and 41BBL, or IL-21 or any combination thereof; or EBV-LCL. In some aspects, the feeder cells provided in combination with a solution of IL-21, IL-15, and/or 41BBL. Feeder cells can be seeded in the culture of cells at a 1:2, 1:1, or 2:1 ratio. It is understood and herein contemplated that the period of culturing can be between 1 and 14 days post AAV infection (i.e., 1, 2, 3, 4, 5, 6, 7, 8, 9, 10, 11, 12, 13, or 14 days), preferably between 3 and 7 days, most preferably between 4 and 6 days.

It is understood and herein contemplated that the incubation conditions for primary cells and expanded cells (including, but not limited to primary and expanded T cells, NK cells, NK T cells, or B cells) can be different. In one aspect, the culturing of primary NK cells or NK T cells prior to AAV infection comprises media and cytokines (such as, for example, IL-2, IL-12, IL-15, IL-18, and/or IL-21) and/or anti-CD3 antibody for less than 5 days (for example 1, 2, 3, or 4 days). For expanded NK cells the culturing can occur in the presence of NK feeder cells (at for example, a 1:1 ratio) in addition to or in lieu of cytokines (such as, for example, IL-2, IL-12, IL-15, IL-18, and/or IL-21) and/or anti-CD3 antibody. Culturing of expanded NK cells can occur for 1, 2, 3, 4, 5, 6, 7, 8, 9, or 10 days prior to transduction. Thus, in one aspect, disclosed herein are methods of genetically modifying a cell (such as for example, a T cell, B cell, macrophage, NK cell, NK T cells, fibroblast, neuronal cell osteoblast, hepatocyte, epithelial cell, and/or muscle cell) comprising incubating primary cells for 4 days in the presence of IL-2 prior to infection with an AAV vector and/or electroporation (when the RNP complex is introduced via electroporation) or incubating expanded cells in the presence of irradiated feeder cells for 4, 5, 6, or 7 days prior to infection with AAV and/or electroporation when the RNP complex is introduced via electroporation.

Following transduction (e.g., via AAV infection or electroporation) of the cell (such as, for example, a T cell, B cell, macrophage, NK cell, NK T cells, fibroblast, osteoblast, hepatocyte, neuronal cell, epithelial cell, and/or muscle cell), the now modified cell can be propagated in a media comprising feeder cells that stimulate the modified cells (such as, for example, a T cell, B cell, macrophage, NK cell, NK T cells, fibroblast, osteoblast, hepatocyte, neuronal cell, epithelial cell, and/or muscle cell). Thus, the modified cells retain viability and proliferative potential, as they are able to be expanded post-AAV infection and/or electroporation (when the RNP complex is introduced via electroporation) using irradiated feeder cells. For example, NK cell stimulating feeder cells for use in the claimed invention, disclosed herein can be either irradiated autologous or allogeneic peripheral blood mononuclear cells (PBMCs) or nonirradiated autologous or PBMCs; RPMI8866; HFWT, K562; K562 cells transfected with membrane bound IL-15, and 41BBL, or IL-21 or any combination thereof; or EBV-LCL. In some aspects, the NK cell feeder cells provided in combination with a solution of IL-21, IL-15, and/or 41BBL. Feeder cells can be seeded in the culture of NK cells at a 1:2, 1:1, or 2:1 ratio. It is understood and herein contemplated that the period of culturing can be between 1 and 14 days post infection and/or electroporation (i.e., 1, 2, 3, 4, 5, 6, 7, 8, 9, 10, 11, 12, 13, or 14 days), preferably between 3 and 7 days, most preferably between 4 and 6 days. In some aspect, the media for culturing the modified NK cells can further comprise cytokines such as, for example, IL-2, IL-12, IL-15, IL-18, and/or IL-21.

In one aspect, it is understood and herein contemplated that one goal of the disclosed methods of genetically modifying a cell is to produce a modified cell. Accordingly, disclosed herein are modified T cells, B cells, macrophages, NK cells, NK T cells, fibroblasts, osteoblasts, hepatocytes, neuronal cells, epithelial cells, and/or muscle cells made by the disclosed methods. Thus, in one aspect, disclosed herein are modified NK cells and/or NK T cells (including, but not limited to CAR NK cells and/or CAR NK T cells) comprising any of the plasmids or vectors disclosed herein. For example, disclosed herein are anti-CD33 CAR NK cells and anti-CD33 CAR NK T cells (including, but not limited to anti-CD33 CAR NK cells and/or NK T cells wherein the anti-CD33 CAR comprises an scFv that targets CD33, a transmembrane domain (such as, for example, a NKG2D transmembrane domain, a CD4 transmembrane domain, a CD8 transmembrane domain, a CD28 transmembrane domain, and/or a CD3ξ transmembrane domain) and a co-stimulatory domain (such as, for example, a 2B4 domain, a CD28 co-stimulatory domain, a 4-1 BB co-stimulatory domain, or any combination of 2B4 domain, a CD28 co-stimulatory domain and/or a 4-1 BB co-stimulatory domain).

In one aspect, disclosed herein are methods of creating a chimeric antigen receptor (CAR) natural killer (NK cell) comprising a) obtaining a ribonucleoprotein (RNP) complex comprising a class 2 CRISPR/Cas endonuclease (Cas9) complexed with a corresponding CRISPR/Cas guide RNA and an AAV vector comprising a plasmid comprising a transgene (such as, for example, a chimeric antigen receptor for a tumor antigen); wherein the transgene is adjacent to one PAM and crRNA or flanked by two PAMs and crRNAs; and b) introducing the transgene and the RNP complex into the cell; wherein the transgene is introduced into the cell via infection with the Adeno-associated virus (AAV) into a target cell; wherein in the ribonucleoprotein (RNP) complex hybridizes to a target sequence within the genomic DNA of the cell, and the cell's DNA repair enzymes insert the transgene into the host genome at the target sequence (for example by non-homologous end joining), thereby creating a modified cell. In one aspect, the method can further comprise introducing the RNP complex into the cell via electroporation (such as when modifying an NK cell or NK T cell). In one aspect, the method can further comprise superinfecting the target cell with a second AAV virus comprising the RNP complex. In one aspect, where the transgene is sufficiently small, the same AAV can comprise both the transgene and the RNP complex. In still further aspect, the transgene and RNP complex can be encoded on the same plasmid.

In some aspect, disclosed herein is a method of genetically modifying a cell comprising a) obtaining a ribonucleoprotein (RNP) complex comprising a class 2 CRISPR/Cas endonuclease (Cas9) complexed with a corresponding CRISPR/Cas guide RNA and an AAV vector comprising a plasmid comprising a polynucleotide sequence encoding a chimeric antigen receptor (CAR) polypeptide; wherein the polynucleotide sequence is flanked by homology arms; and wherein the homology arms are 800 bp in length or less; and b) introducing the polynucleotide sequence and the RNP complex into the cell; wherein the polynucleotide sequence is introduced into the cell via infection with the AAV into the cell; wherein the RNP complex hybridizes to a target sequence within the genomic DNA of the cell and the cell's DNA repair enzymes insert the transgene into the host genome at the target sequence within the genomic DNA of the cell thereby creating a modified cell. In some embodiments, the cell is an NK cell.

In one aspect, the modified cells (e.g., NK cells) used in the disclosed immunotherapy methods and created by the disclosed modification methods can be primary cells from a donor source (such as, for example, an allogeneic donor source for an adoptive transfer therapy or an autologous donor source (i.e., the ultimate recipient of the modified cells), a cell line (including, but not limited to NK cell lines NK RPMI8866; HFWT, K562, and EBV-LCL), or from a source of expanded cells derived a primary cell source or cell line. Because primary cells can be used, it is understood and herein contemplated that the disclosed modifications of the cell can occur ex vivo or in vitro.

The cells used herein can be primary cell or expanded cells. The primary cells may be incubated for about 4 to 10 days in the presence of IL-2 prior to infection of AAV vectors. In one example, the primary cells are expanded for about 4 to 10 days in the presence of irradiated feeder cells, plasma membrane particles, or exosomes prior to infection. In some embodiments, the irradiated feeder cells, plasma membrane particles, or exosomes express membrane bound 4-1BBL, membrane-bound IL-21, or membrane-bound-15 or any combination thereof.

Following transduction of the cells (e.g., NK cells), the modified cells can be expanded and stimulated prior to administration of the modified (i.e., engineered) cells to the subject. For example, disclosed herein are methods of adoptively transferring immune cells to a subject in need thereof wherein the immune cell (e.g., natural killer (NK) cell) is expanded with irradiated feeder cells, plasma membrane (PM) particles, or exosomes (EX) expressing membrane bound IL-21 (mbIL-21) (PM particles and EX exosomes expressing mbIL-21 are referred to herein as PM21 particles and EX21 exosomes, respectively) prior to administration to the subject. In some aspects, expansion can further comprise irradiated feeder cells, plasma membrane (PM) particles, or exosomes expressing membrane bound IL-15 (mblL-15) and/or membrane bound 4-1BBL (mb4-1BBL). In some aspects, it is understood and herein contemplated that the stimulation and expansion of the modified (i.e., engineered) cells can occur in vivo following or concurrent with the administration of the modified cells to the subject. Accordingly disclosed herein are immunotherapy methods wherein the cells (e.g., NK cells) are expanded in the subject following transfer of the cells to the subject via the administration of IL-21 or PM particles with mblL-21, exosomes with mblL-21, and/or irradiated mblL-21 expressing feeder cells. In some aspect, the expansion further comprises the administration of IL-15 and/or 4-1BBL or PM particles, exosomes, and/or irradiated feeder cells that express membrane bound IL-15 and/or 4-1BBL.

In some embodiments, the method disclosed herein comprises infecting the NK cell with a range of MOI of AAV from about 1 to about 1000K MOI (e.g., about 5 to 500K MOI) of AAV. For example, the method disclosed herein comprises infecting the NK cell with at least about 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 100, 150, 200, 250, 300, 350, 400, 450, or 500 MOI of AAV.

1. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000-fold excess. This type of assay can be performed at under conditions where both the limiting and non-limiting primer are for example, 10-fold or 100-fold or 1000-fold below their k_{d}, or where only one of the nucleic acid molecules is 10-fold or 100-fold or 1000-fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

2. Nucleic Acids

There are a variety of molecules disclosed herein that are nucleic acid based. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or 0) at the C6 position of purine nucleotides.

b) Sequences

There are a variety of sequences related to the protein molecules involved in the signaling pathways disclosed herein, for example CD33, 4-1BB, NKG2D, or 2B4, all of which are encoded by nucleic acids or are nucleic acids. The sequences for the human analogs of these genes, as well as other analogs, and alleles of these genes, and splice variants and other types of variants, are available in a variety of protein and gene databases, including Genbank. Those of skill in the art understand how to resolve sequence discrepancies and differences and to adjust the compositions and methods relating to a particular sequence to other related sequences. Primers and/or probes can be designed for any given sequence given the information disclosed herein and known in the art.

c) Primers and Probes

Disclosed are compositions including primers and probes, which are capable of interacting with the disclosed nucleic acids, such as the CD33 as disclosed herein. In certain embodiments the primers are used to support DNA amplification reactions. Typically the primers will be capable of being extended in a sequence specific manner Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the CD33 gene typically will be used to produce an amplified DNA product that contains a region of CD33 gene or the complete gene. In general, typically the size of the product will be such that the size can be accurately determined to within 3, or 2 or 1 nucleotides.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

3. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). In some examples, the plasmid descried herein can be a DNA template or a nucleotide construction that comprises the polynucleotide sequences provided herein.

As used herein, plasmid or viral vectors are agents that transport the disclosed nucleic acids into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19 (such as, for example at AAV integration site 1 (AAVS1)). Vectors which contain this site-specific integration property are preferred. AAVs used can be derived from any AAV serotype, including but not limited to AAC1, AAV2, AAV3, AAV4, AAV5, AAV6, AAV7, AAV8, AAV9, and recombinant (rAAV) such as, for example AAV-Rh74, and/or synthetic AAV (such as, for example AAV-DJ, Anc80). AAV serotypes can be selected based on cell or tissue tropism. AAV vectors for use in the disclosed compositions and methods can be single stranded (SS) or self-complementary (SC).

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically, the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

It is understood and herein contemplated that the packaging capacity of an AAV is limited. One method to overcome the loading capacity of an AAV vector is through the use of two vectors, wherein the transgene is split between the two plasmids and a 3′ splice donor and 5′ splice acceptor are used to join the two sections of transgene into a single full-length transgene. Alternatively, the two transgenes can be made with substantial overlap and homologous recombination will join the two segments into a full-length transcript.

4. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g., beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

Peptides

a) Protein Variants

Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 5 and 6 and are referred to as conservative substitutions.

TABLE 5
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 6
Amino Acid Substitutions Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 6, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 5 and Table 6. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

6. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

7. Method of Treating Cancer

The plasmids, vectors, and modified NK cells and NK T cells disclosed herein can be used to treat, inhibit, reduce, decrease, ameliorate, and/or prevent any disease where uncontrolled cellular proliferation occurs such as cancers. Cancer immunotherapy has been advanced in recent years; genetically-modified chimeric antigen receptor (CAR) T cells are an excellent example of engineered immune cells successfully deployed in cancer immunotherapy. These cells were recently approved by the FDA for treatment against CD19+B cell malignancies, but success has so far been limited to diseases bearing a few targetable antigens, and targeting such limited antigenic repertoires is prone to failure by immune escape. Furthermore, CAR T cells have been focused on the use of autologous T cells because of the risk of graft-versus-host disease (GvHD) caused by allogeneic T cells. In contrast, NK cells are able to kill tumor targets in an antigen-independent manner and do not cause GvHD, which makes them a good candidate for cancer immunotherapy. It is understood and herein contemplated that the disclosed plasmids and methods can be used to generate, for example, CAR NK T cells and CAR NK cells to target a cancer.

Thus, disclosed herein are methods of treating, decreasing, reducing, inhibiting, ameliorating, and/or preventing a cancer and/or metastasis (such as, for example, acute lymphocytic leukemia (ALL), acute myeloid leukemia (AML), chronic myeloid leukemia (CML), hairy cell leukemia (HCL), and/or myelodysplastic syndromes (MDS)) in a subject comprising administering to a subject with a cancer any modified cell (for example, modified NK cells and NK T cells) disclosed herein. For example, disclosed herein are methods of treating, decreasing, reducing, inhibiting, ameliorating, and/or preventing a cancer and/or metastasis (such as, for example, acute lymphocytic leukemia (ALL), acute myeloid leukemia (AML), chronic myeloid leukemia (CML), hairy cell leukemia (HCL), and/or myelodysplastic syndromes (MDS)) in a subject comprising administering to the subject a therapeutically effective amount of a natural killer (NK) cell or NK T cell, wherein the NK cell or NK T cell comprises a plasmid for use with clustered regularly interspaced short palindromic repeat (CRISPR)/CRISPR-associated 9 (Cas9) integration systems wherein the plasmid comprises in order a left homology arm, a polynucleotide sequence encoding a chimeric antigen receptor (CAR) polypeptide (such as, for example a CD33 targeting CAR), and a right homology arm; wherein the left and right homology arms are each 1000 bp in length or less (for example, 600 bp).

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

As noted above, the plasmids, vectors, and modified NK cells and NK T cells disclosed herein can be used to treat, inhibit, reduce, decrease, ameliorate, and/or prevent cancer. A representative but non-limiting list of cancers that the disclosed compositions can be used to treat is the following: lymphoma, B cell lymphoma, T cell lymphoma, mycosis fungoides, Hodgkin's Disease, acute lymphocytic leukemia (ALL), hairy cell leukemia (HCL), myelodysplastic syndromes (MDS), myeloid leukemia (including, but not limited to acute myeloid leukemia (AML) and chronic myeloid leukemia (CML)), bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

As noted throughout the present disclosure, the disclosed modified NK cells are ideally suited for use in immunotherapy such as the adoptive transfer of modified (i.e, engineered NK cells to a subject in need thereof). Thus, in one aspect, disclosed herein are methods of adoptively transferring an engineered NK cells to a subject in need thereof said method comprising a) obtaining an NK cell to be modified; b) obtaining a ribonucleoprotein (RNP) complex comprising a class 2 CRISPR/Cas endonuclease (Cas9) complexed with a corresponding CRISPR/Cas guide RNA and an AAV vector comprising a plasmid comprising a transgene (such as, for example, a chimeric antigen receptor for a tumor antigen); wherein the transgene is flanked by homology arms; and wherein the homology arms are less than 1000 bp; and c) introducing the transgene and the RNP complex into the NK cell; wherein the transgene is introduced into the cell via infection with the Adeno-associated virus (AAV) into the NK cell; wherein the RNP complex hybridizes to a target sequence within the genomic DNA of the NK cell and the NK cell's DNA repair enzymes insert the transgene into the host genome (for example, by homologous repair) at the target sequence within the genomic DNA of the target cell thereby creating an engineered NK cell; and d) transferring the engineered NK cell into the subject. In one aspect the transgene can be comprised on the same plasmid as the Cas9 endonuclease or encoded on a second plasmid in the same or different AAV vector. In one aspect, the target cell can be transduced with the RNP complex via electroporation before or concurrently with the infection of the cell with the transgene comprising AAV.

In one aspect, the modified cells cell (e.g., NK cells) used in the disclosed immunotherapy methods can be primary cells from a donor source (such as, for example, an allogeneic donor source for an adoptive transfer therapy or an autologous donor source (i.e., the ultimate recipient of the modified cells), a cell line (including, but not limited to NK cell lines NK RPMI8866; HFWT, K562, and EBV-LCL), or from a source of expanded cells derived a primary cell source or cell line. Because primary cells can be used, it is understood and herein contemplated that the disclosed modifications of the cell can occur ex vivo or in vitro.

Also disclosed herein is a plasmid comprising in order a left homology arm, a polynucleotide sequence encoding a chimeric antigen receptor (CAR) polypeptide, and a right homology arm; wherein the left and right homology arms are each 1000 bp in length or less.

In another aspect, disclosed herein are a plasmid, an AAV vector or a modified cell as disclosed herein for use as a medicament. Also disclosed herein are a use of a plasmid, an AAV vector or a modified cell as disclosed herein for the manufacture of a medicament.

Also disclosed herein are a plasmid, an AAV vector or a modified cell as disclosed herein for use in the treatment of cancer. Also disclosed herein are a use of a plasmid, an AAV vector, or a modified cell as disclosed herein for the manufacture of a medicament for the treatment of cancer.

Also disclosed herein are a CAR NK cell, created by using a method of creating a chimeric antigen receptor (CAR) natural killer (NK) cell or NK T cell as disclosed herein, for use in the treatment of cancer. Also disclosed herein are a use of a CAR NK cell, created by using a method of creating a chimeric antigen receptor (CAR) natural killer (NK) cell or NK T cell as disclosed herein, for the manufacture of a medicament for the treatment of cancer.

### VI. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Highly Efficient Site-Directed Gene Insertion in Primary Human Natural Killer Cells Using Homologous Recombination and CRISPaint Delivered by AAV6

Using the approaches described herein, highly efficient and stable transgene-modified human primary NK cells were successfully generated, including two CAR-NK cells which showed enhanced anti-AML activity.

a) Methods

(1) Human NK Cell Purification and Expansion.

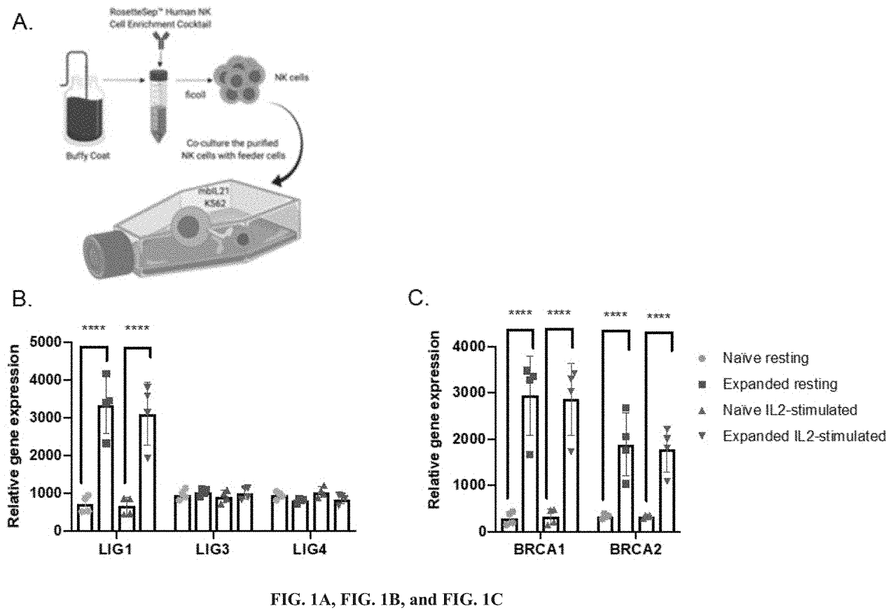

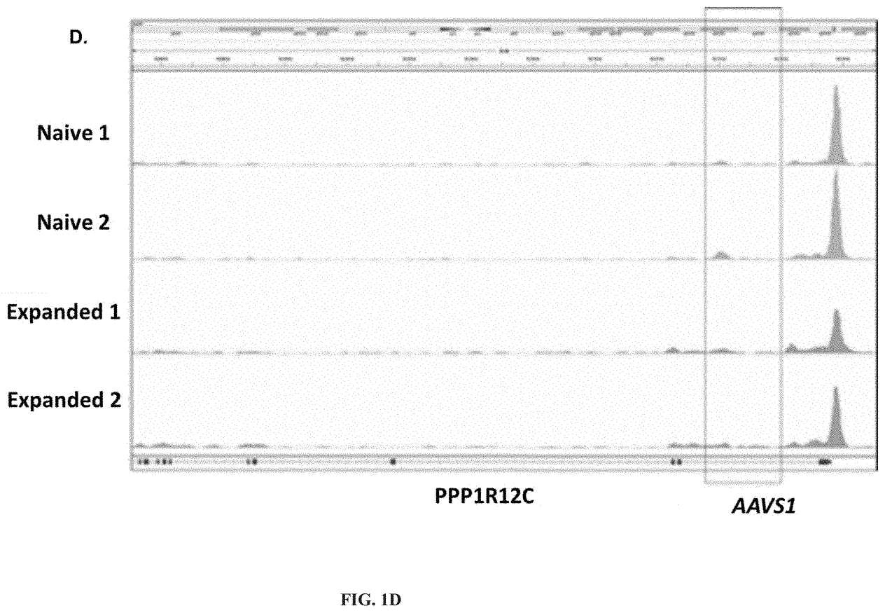

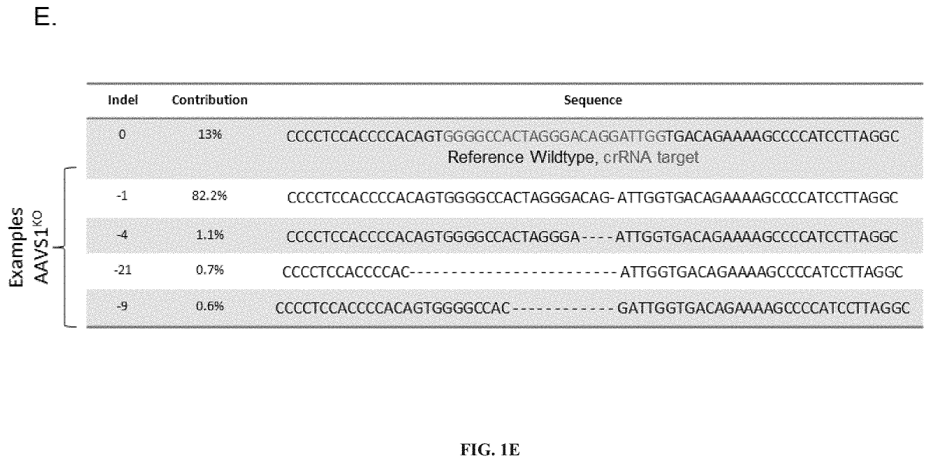

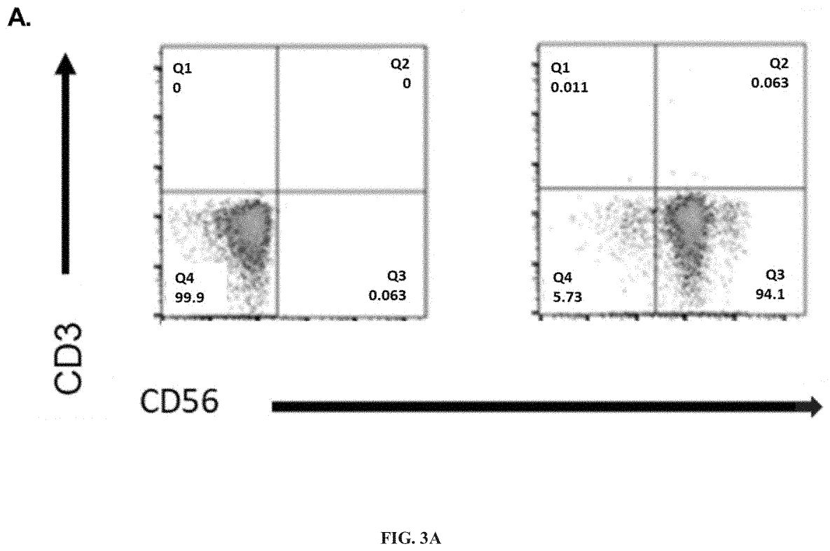

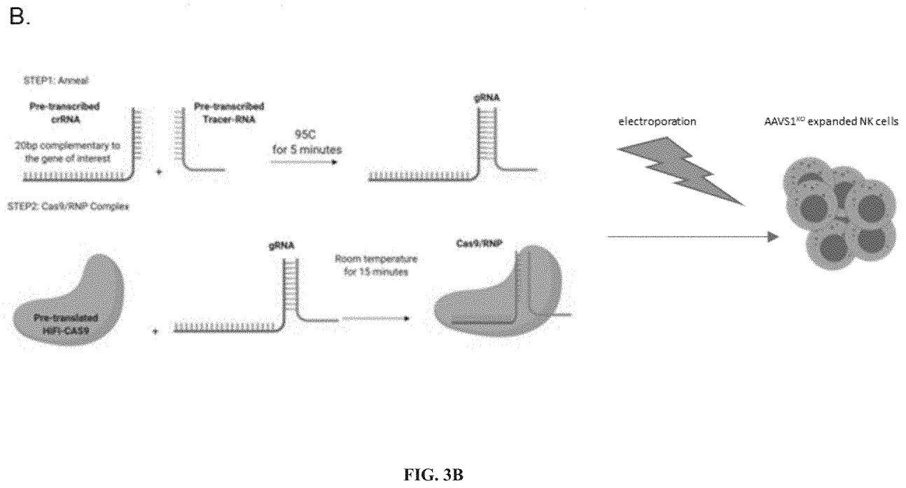

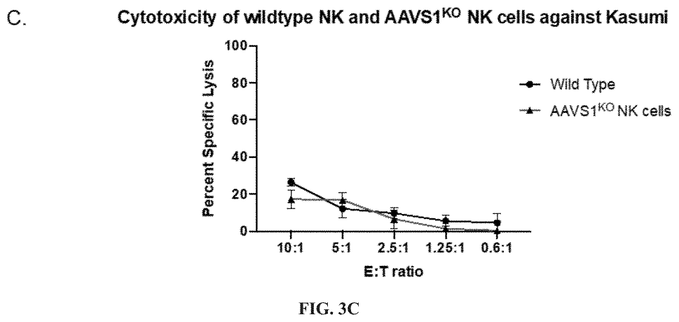

NK cells were purified as previously described. Briefly, NK cells were isolated from PBMC collected from healthy individuals using RosetteSep™ Human NK Cell Enrichment Cocktail (FIG. 1A). Purified NK cells were phenotyped using flow cytometry as >90% CD3-negative/CD56-positive population (FIG. 3A). These cells were stimulated with irradiated K562 feeder cells expressing 4-1BBL and membrane-bound IL-21 (FC21) at a ratio of 2:1 (feeder: NK) at the day of purification (FIG. 1A). The stimulated cells were cultured for 7 days in the serum-free AIM-V/ICSR expansion medium containing 50 IU/mL of IL-2.

(2) ATAC-Seq Assay.

Freshly-isolated (naïve), FC15-, and FC21-expanded NK cells were cryopreserved in aliquots of 100,000 viable cells/vial before processing for ATAC-seq. ATAC-seq was performed as previously described. DNA libraries were sequenced using Illumina HiSeq 2500 at 50 bp paired-end reads.

(3) Cas9/RNP Electroporation for Targeting AAVS1 in NK Cells.

AAVS1 was targeted using one gRNA (crRNA: 5′ GGGGCCACTAGGGACAGGAT) (SEQ ID NO: 17) via electroporation of Cas9/RNP into day seven expanded NK cells as described before. Briefly, 3×10⁶ expanded NK cells were harvested and washed twice with 13 ml of PBS followed by centrifugation for 5 minutes at 400 g and aspiration of PBS. The cell pellet was resuspended in 20 ul of P3 Primary Cell 4D-Nucleofector Solution. 5 ul of pre-complexed Cas9/RNP (ALT-R® CRISPR-Cas9 crRNA, ALT-R® CRISPR-Cas9 tracrRNA, and ALT-R® S.p. HiFi Cas9 Nuclease V3) (Integrated DNA Technologies, Inc., Coralville, Iowa), targeting AAVS1 and 1 ul of 100 uM electroporation enhancer (ALT-R® Cas9 Electroporation Enhancer) were added to the cell suspension. The total volume of 26 ul of CRISPR reaction was transferred into 4D-Nucleofector™ 16-well Strip and electroporated using program EN-138 (FIG. 3B). After electroporation, the cells were transferred into 2 ml of media containing 50 IU of IL-2 in a 12 well plate and incubated at 37 degrees and 5% CO₂ pressure. Two days post electroporation, cells were stimulated with 2×10⁶ feeder cells, and 8 ml fresh media complemented with 50 IU was added in cell suspension and kept in a T25 flask.

(4) ICE Mutation Detection Assay.

To measure the indel rate in AAVS1KO NK cells, PCR was used to amplify the Cas9/RNP targeting site using forward and reverse primes mentioned in Table 1. The amplicons were sequenced using sanger sequencing, and results were analyzed using ICE.

(5) RNA-Seq Sample Preparation and Sequencing.

Total RNA was purified from naïve resting, expanded resting, naïve IL-21-stimulated, and day seven FC21-expanded NK cells using the Total RNA Purification Plus Kit (Norgen Biotek, Ontario, Canada). The resulting total RNA was quantified in a Nanodrop ND-1000 spectrophotometer, checked for purity and integrity in a Bioanalyzer-2100 device (Agilent Technologies Inc., Santa Clara, Calif.) and submitted to the genomics core at the Nationwide Children's Hospital for sequencing. Libraries were prepared using the TruSeq RNA Sample Preparation Kit (Illumina Inc.) according to the protocols recommended by the manufacturer. Library quality was determined via Agilent 4200 Tapestation using a High Sensitivity D1000 ScreenTape Assay kit and quantified by KAPA qPCR (KAPA BioSystems). Approximately 60-80 million paired-end 150 bp sequence reads per library were generated using the Illumina HiSeq4000 platform.

Sequencing reads from each sample were aligned to the GRCh38.p9 assembly of the Homo sapiens reference from NCBI using version 2.5.2b of the splice-aware aligner STAR. Feature coverage counts were calculated with HTSeq, using the GFF file that came with the assembly from NCBI. The default options for feature type, exon, and feature identifier, gene id, from the GFF were used to identify features for RNA-Seq analysis. Quality control checks for sample preparation and alignment were performed using custom Perl scripts, which count types of reads using STAR's mapping quality metric and the number of reads aligned to each feature class defined by the feature table that came with the assembly from NCBI.

(6) AAV6 Production.

The transgenes cloned into ssAAV or scAAV plasmids were packaged in AAV6 capsids as described before.

(7) Combining Cas9/RNP and AAV6 to Generate mCherry and CAR NK Cells.

A media change and resuspension at 5×10⁵ cells per ml were performed on day 6 of NK cell expansion one day before experimental manipulation. The NK cells were then electroporated with Cas9/RNP targeting AAVS1 on day 7, as described above. Thirty minutes after electroporation, 3×10⁵ live cells were collected and resuspended at 1×10⁶ cells per ml in media containing 50 IU IL2 (Novartis) in a 24 well plate in a total volume of 300 ul. For each transduction condition with ssAAV6 or scAAV6 to deliver HR or CRISPaint DNA encoding mCherry or CD33CARs, we transduced 3×10⁵ electroporated cells with 300K MOI (10-500K MOI if needed). Negative controls included as NK cells that were not electroporated were electroporated with Cas9/RNP but not AAV transduced or were transduced with 300K MOI of AAV6 without electroporation of Cas9/RNP. The day after electroporation and transduction, we added 300 ul of fresh media containing 50 IU of IL2 to each well without changing the old media. The cells were kept in culture for 48 hours after electroporation and were then restimulated with 2×10⁶ feeder cells and kept in a total volume of 2 ml media containing 50 IU in 12 well plate, without changing the old media. 48 hours later, 8 ml fresh media supplemented with IL2 was added to cells, a total volume of 10 ml was kept in a T25 flask. At day 7 post-transduction, cells were re-stimulated with feeder cells at a ratio of 1:1 and grown for one more week, every 2 days fresh media was added to the cells.

(8) Flow Cytometry for Detection of CAR-NK Cells.

7 days and 14 days following electroporation, 5×10⁵ NK cells were washed twice with staining buffer containing 2% FBS in PBS. Next, 2.5 ug of recombinant human siglec-3/CD33 Fc chimera protein, (CF; R&D systems #1137-SL-050) was added to cell suspension in a total volume of 80 ul and incubated for 30 minutes at 4 C. Cells were washed twice with staining buffer before staining with 2 ul of Alexa Fluor® 647 affinipure goat anti-human IgG, Fcγ fragment specific, (Jackson ImmunoResearch #109-605-098) at 1:100 ratio in 200 ul of staining buffer and kept at 4 C for 30 minutes. Once stained, cells were washed twice with staining buffer then acquired on MacsQuant flow cytometers. Flow cytometry data were analyzed using FlowJo software (FlowJo, LLC).

(9) Cytotoxicity Assay.

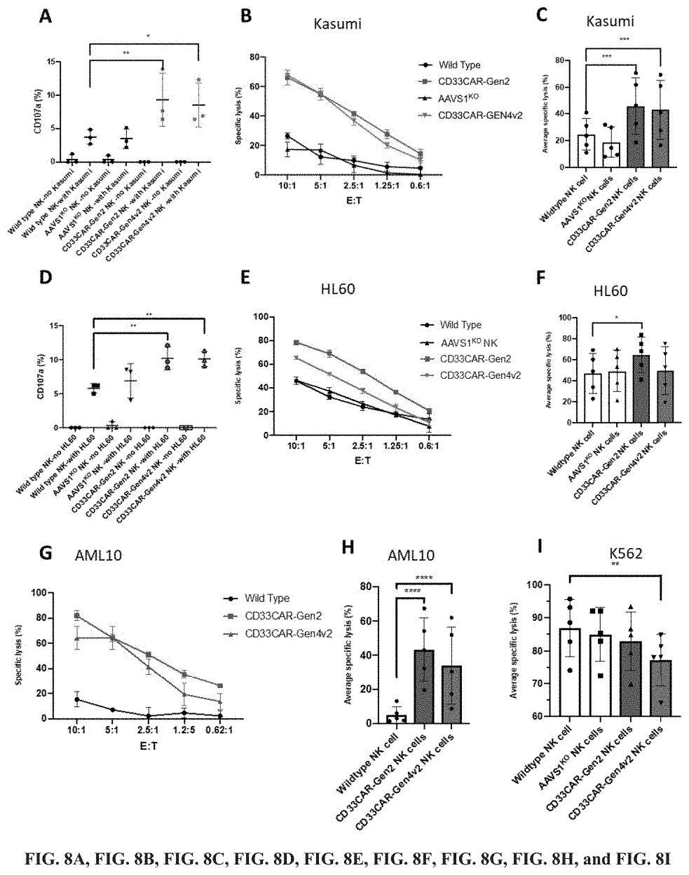

Cytotoxicity assays were performed for 3-4 h as described previously using a calcein-acetoxymethyl-release assay. Cytotoxicity was assessed against Kasumi-1, HL60, or AML10 cells at different ratios of target: effector as defined in FIG. 8.

(10) CD107a Staining.

NK cells and cancer cells were cocultured at 10:1 ratio and supplemented with 20 ul of PE mouse anti-human CD107a antibody (BD Pharmingen™, #555801) in a total volume of 220 ul in a 96 well plate. We kept the plate at 37 C incubator for 90 minutes. Then, the cells were washed with staining buffer once and collected for acquiring on MacsQuant flow cytometers.

(11) PCR-Based Detection of Transgenes Integration.

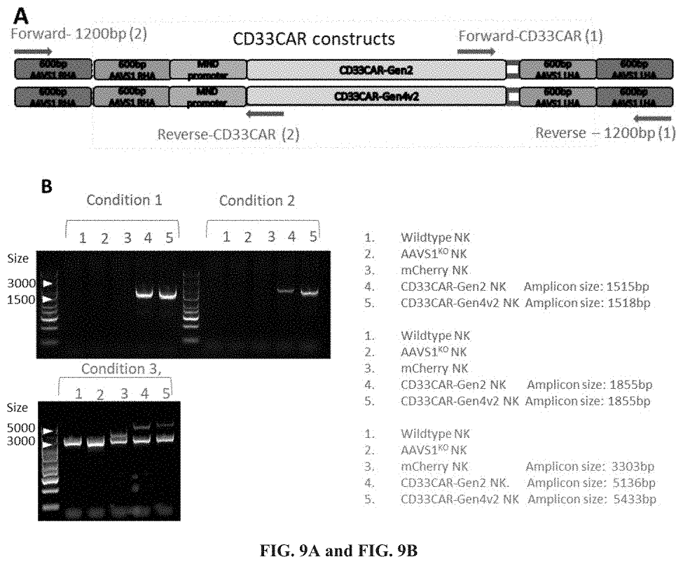

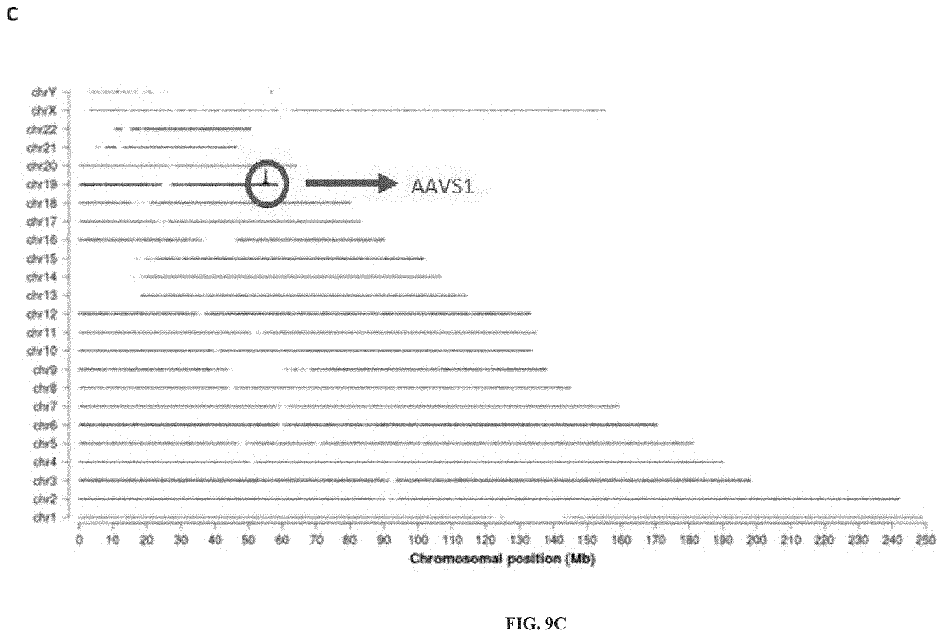

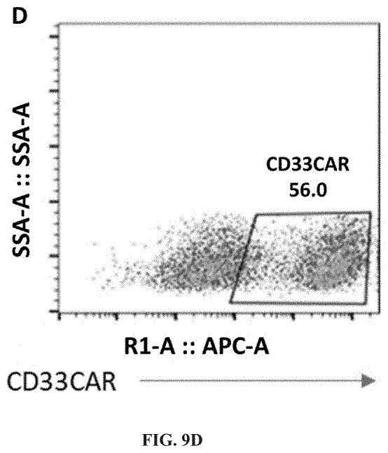

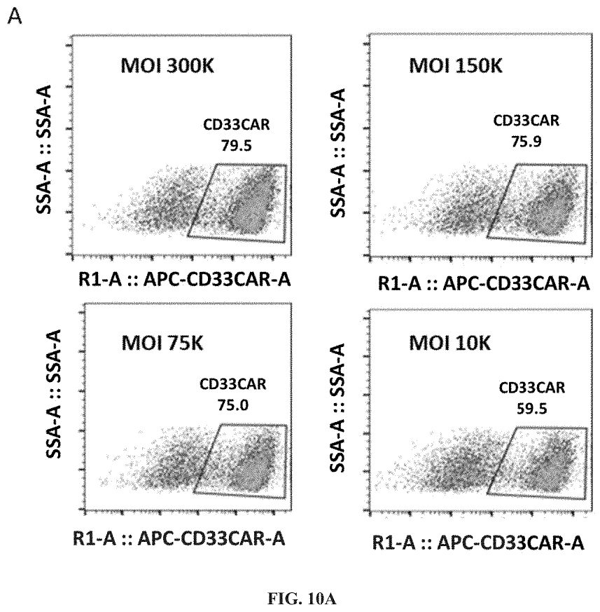

In-out PCR was performed using 2 pairs of primers (FIGS. 9A and 9B and Table 2) designed inside or outside of the CD33CAR constructs. We also added a set of primers to amplify 1200 bp right and left flanking region of Cas9 targeting and transgene integration site (FIG. 9C). PCRs were performed using the Platinum™ Taq DNA polymerase high fidelity kit (Thermofisher #11304011).

TLA. For the whole-genome mapping of CD33CAR-Gen2 integration, we used the TLA technology (Cergentis B.V.). For details, see FIG. 9C.

b) Results

(1) Expansion of NK Cells Using FC21 Provides Optimal Condition for Gene Insertion.

Enzymatic reactions regulate CRISPaint and HR. CRISPaint is a LIG4-dependent process, while other proteins such as BRCA1 and BRCA2 regulate HR. Therefore, the expression level of these genes were analyzed in NK cells freshly isolated or seven days after stimulation with feeder cells expressing membrane-bound IL-21 (FC21) (n=4) to evaluate which repair pathway was more efficient in this cell type and in which stage of expansion (FIG. 1A, FIGS. 3A and 3B). RNA-seq analysis showed that the day seven expanded NK cells have higher expression of BRCA1 and BRCA2 in comparison to naïve NK cells. Additionally, there is no decrease in LIG4 level in these cells; however the level of LIG1, which is a DNA-repair enzyme, was significantly higher in expanded cells (FIGS. 1B and 1C), providing optimal conditions for either HR or NHEJ-directed gene insertion through CRISPaint in day 7 expanded NK cells.

(2) Successful Targeting of the Genomic Safe Harbor for Gene Insertion.

Genomic safe harbors (GSHs) are sites in the genome that can be modified with no change in the normal function of the host cell and allow adequate expression of the transgene. For gene insertion in NK cells, the adeno-associated virus site 1 (AAVS1) was chosen, which is one of the GSHs and an exemplary locus within the phosphatase 1 regulatory subunit 12C (PPP1R12C) gene. This locus has been successfully used for directed gene insertion into several cell types. First, the chromatin accessibility of AAVS1 in naïve and expanded NK cells (n=2) was evaluated by ATAC-seq assay and showed no reduction in chromatin accessibility in FC21-expanded NK cells in comparison to naïve NK cells (FIG. 1D). Next, AAVS1 was targeted using one gRNA via electroporation of Cas9/RNP into day seven expanded NK cells. After 48 hours, NK cell DNA was isolated for detection of Insertions deletions (Indels) in CRISPR edited NK cells using Inference of CRISPR Edits (ICE) to analyze the frequency of Indels. The ICE results showed that up to 85% of CRISPR modified NK cells had at least one indel at the AAVS1 Cas9-targeting site (FIG. 1E). To ensure that genome modifications at this locus did not interfere with the ability of NK cells to target cancer cells, the cytotoxicity of AAVS1KO NK cells was assessed against Kasumi-1, an acute myeloid leukemia (AML) cancer cell line. Using a Calcein AM assay, no difference between wild type and CRISPR modified NK cells in their killing ability was observed (FIG. 3C).

(3) Successful Generation of mCherry Expressing Primary Human NK Cells Using a Combination of Single-Stranded AAV6 and Cas9/RNP.

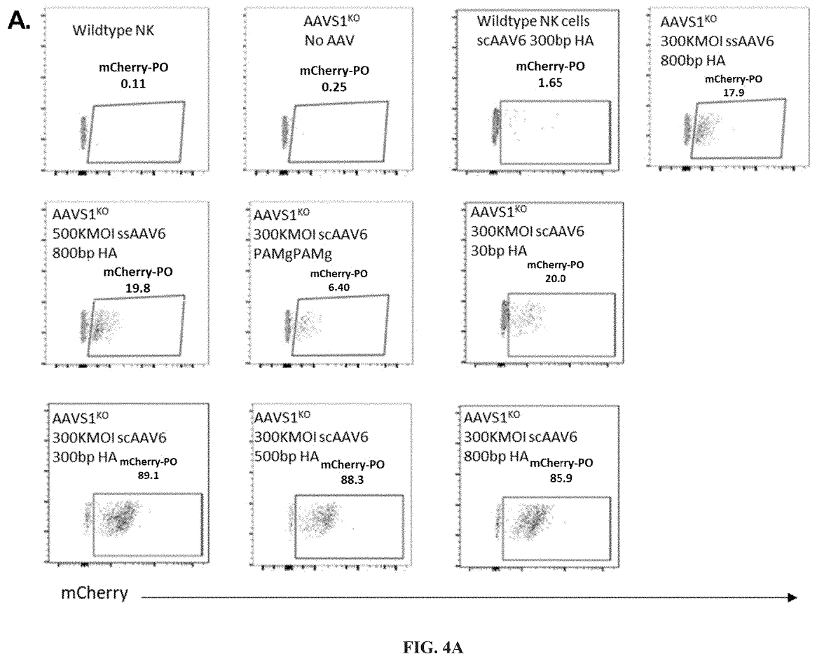

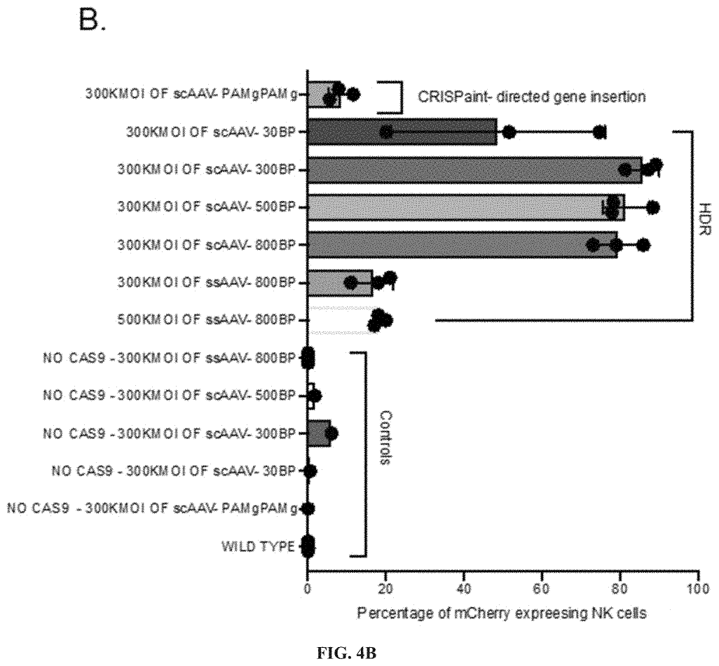

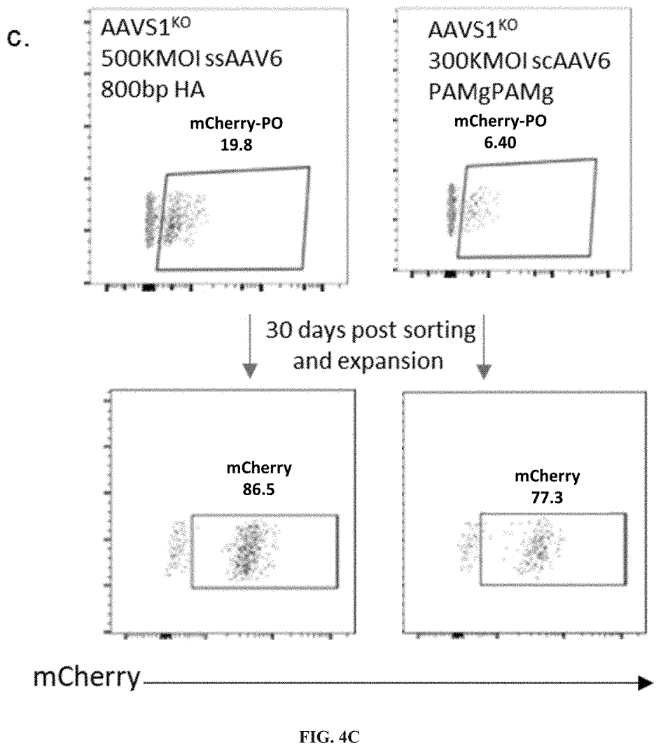

For HDR-mediated gene insertion, DNA-encoding mCherry with 800 bp HA for the right and 1000 bp for the left site flanking region of cas9 targeting site in AAVS1 locus was cloned into the backbone of single-stranded AAV plasmid and packaged into the AAV6 viral capsid. The constructs were designed to have a splice acceptor downstream of the transgene to improve the transcription of the mCherry gene (FIG. 2A). As described in the methods, the NK cells were electroporated with Cas9/RNP targeting AAVS1, and after half an hour, the cells were transduced with 300K MOI or 500K MOI of AAV6 (FIG. 2C). This resulted in generating 17% (300K MOI) and 19% (500K MOI) mCherry positive NK cells, evaluated 48 hours post electroporation using flow cytometry. These cells were further expanded for one week using FC21 and enriched the mCherry positive cells by FACS sorting. This resulted in an enriched population of mCherry positive NK cells (77% mCherry positive NK cells transduced with 300K MOI, and 86% for the NK cells transduced with 500K MOI of ssAAV6). These cells were restimulated using feeder cells and expanded for another 30 days and no reduction in the expression level of mCherry was observed (FIGS. 4A, 4B, and 4C).

(4) Improved Gene Insertion by Using Self-Complementary AAV6 and Cas9/RNP.

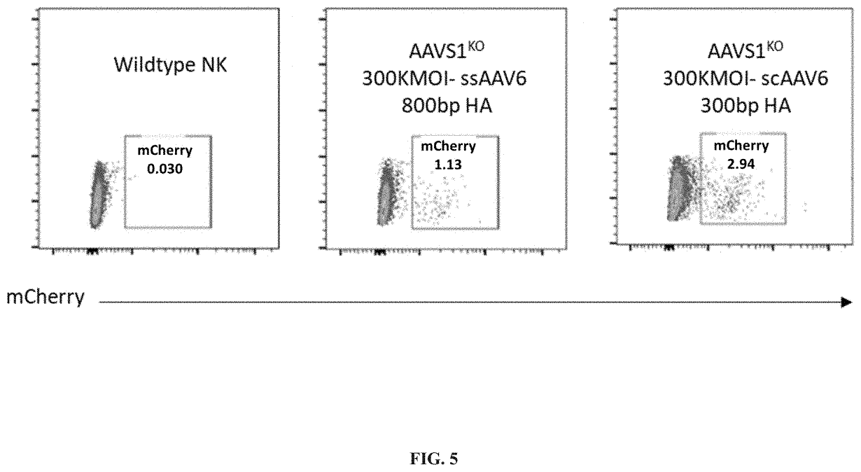

As described earlier, scAAV vectors can become double-stranded in a shorter time frame in comparison to ssAAV, after entering into the host cells. It may increase the efficiency of gene insertion in NK cells. To test this, scAAV6 and combine them with Cas9/RNP was used to improve the gene insertion outcome of the ssAAV6 method. Due to the size limitation of packaging transgenes in scAAV, several lengths of HAs were designed to provide a wide range of possibilities for cloning transgenes with different sizes into scAAV backbones. Hence, DNA encoding mCherry with 30 bp, 300 bp, 500 bp, and 1000 bp of HA for the right and 30 bp, 300 bp, 500 bp, and 800 bp for the left HA (FIG. 2A) were cloned into the scAAV backbone and packaged into AAV6 capsid. The same steps as described earlier were then followed for the ssAAV section to electroporate and transduce the day 7 expanded NK cells. This approach significantly increased the efficiency of generating mCherry expressing NK cells, with the positive percentages reported as follows: 30 bp (19-20%), 300 bp (80-85%), 500 bp (75-85%), and 800 bp (80-89%) (FIGS. 4A and 4B). These cells can be further expanded using feeder cells for more than 3 weeks and did not see any drop in the percentage of mCherry expressing NK cells, showing stable exogenous gene expression. Although, due to the size limitation in scAAV, these vectors cannot be used for generating CAR NK cells, mCherry can be considered as a proof of concept for generating NK cells with the ability to produce highly efficient and stable exogenous proteins. When the same approach of Cas9/RNP electroporation and AAV6 transduction was used in freshly isolated NK cells, the percentages of mCherry expression were significantly low (%1.13 for ss800 bp AAV6 and %2.9 for sc300 bp AAV6, FIG. 5). Based on these observations, FC21-expanded NK cells were used.

(5) CRISPaint can be Used for Gene Insertion in NK Cells.

To overcome the complexity of HAs optimization seen in HDR directed gene insertion, a homology independent gene insertion approach called CRISPaint was tested. For the CRISPaint DNA templates, double Cas9-targeting sequences of AAVS1 (PAMgPAMg) were incorporated around the mCherry transgene but within the ITRs of scAAV and packaged it into AAV6 (FIG. 2B). The methods used for electroporation and transduction of NK cells for HR directed gene insertion were also performed here. Two days after electroporation and transduction and before expansion, flow cytometry was performed to assess mCherry expression in NK cells. The cells which were electroporated and transduced with 300K MOI of scAAV6 delivering CRISPaint PAMgPAMg were found to be up to 6% of mCherry positive. These cells can be further sorted out and enriched up to 77% mCherry expressing NK cells and expanded using FC21 for 30 days and saw no decline in the percentage of mCherry positive NK cells (FIGS. 4B and 4C). Although lower efficiency of gene integration using CRISPaint was seen compared to HR-directed gene insertion, this method is still desirable because it allows researchers to integrate genes of interest into a user-defined locus with no need for designing homology arms.

(6) Successful Generation of Human Primary CD33 CAR NK Cells.

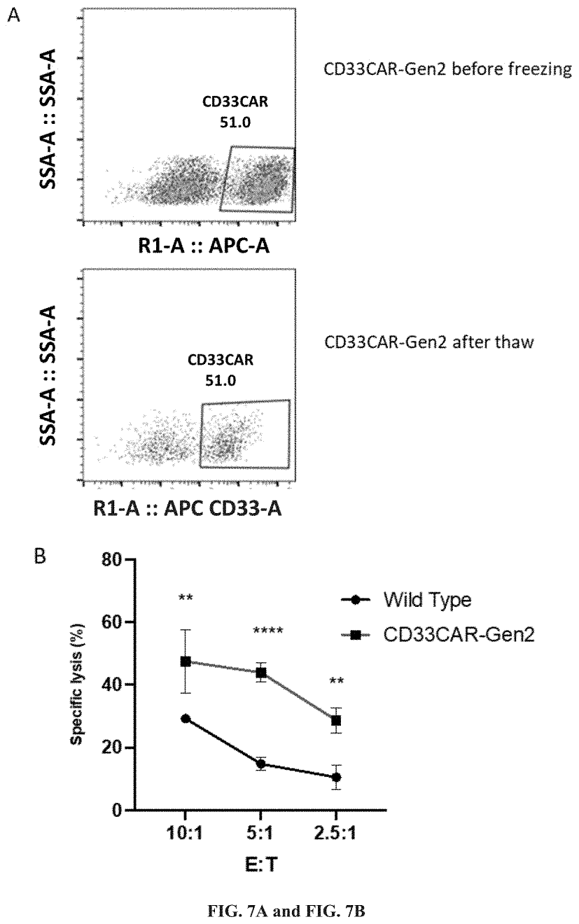

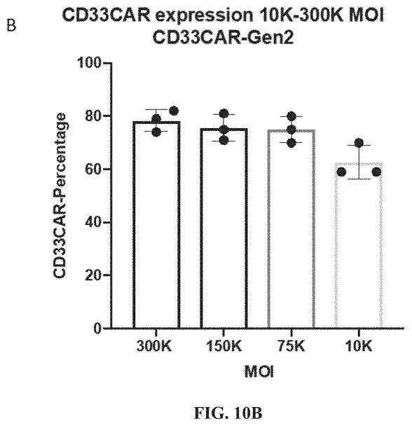

To generate the CD33 targeting CAR NK cells, two constructs (Gen2 and Gen4v2) were designed. The CARs used here contain the same scFv derived from CD33 monoclonal antibody followed by CD4 and CD28 as co-stimulatory domains, alongside CD3z for Gen2 and NKG2D, 2B4 followed by CD3z for Gen4v2 (FIGS. 6A and 6B). To improve the expression level of the CARs, which is larger than mCherry, instead of using splice acceptor, a murine leukemia virus-derived (MND) was incorporated, which is a highly and constitutively active promoter in the hematopoietic system before the starting codon of the CARs. The DNA encoding CD33CARs were then cloned with 600 bp HAs for the AAVS1 targeting site into a backbone of ssAAV and packaged them into the AAV6 capsid. Seven days post electroporation and transduction, the CAR expression on NK cells was analyzed using flow cytometry and up to 78% positive CD33 CAR-expressing NK cells was detected (mean 59.3% for Gen2 and 60% for Gen4v2 at day 14 post transduction). Higher mean florescent intensity (MFI) of CD33CAR-Gen2 expressed on NK cells was observed in comparison to Gen4v2 (FIGS. 6C and 6D). Next, the cells were expanded and grew on feeder cells for another week (Day 14) and no significant reduction in expression of CARs was shown between day 7 and day 14 CAR-NK cells (FIG. 6E). The gene manipulation also did not have any significant effect on the expansion of the CAR-expressing cells in comparison to wildtype cells (FIG. 6F). Freeze and thaw process also did not have any negative impact on CAR expression and the enhanced cytotoxicity of CAR NK cells (FIGS. 7A and 7B). Next, using PCR confirmed the integration of the DNA encoding transgenes at the DNA level (FIGS. 9A and 9B). Additionally, targeted locus amplification (TLA) technology was used for whole-genome mapping of CD33CAR-Gen2 integration with a sensitivity of detecting random integration of more than 5% and demonstrated that the vector integrated correctly at the targeted location in chromosome 19 in a subset of the sample. There are no indications for abundant off-target integration sites. In the sample 1 sequence variant and 4 structural variants were detected which indicate that at least in a subset of the sample a incorrect targeting event took place. In addition also random integrations were identified in chr19 in a subset of the sample (FIG. 9C). It was also shown that decreasing the virus concentration to 10K MOI also can be used for CD33CAR-Gen2 NK cell production (FIGS. 10A and 10B).

(7) Human Primary CAR-NK Cells have Enhanced Antitumor Activity.

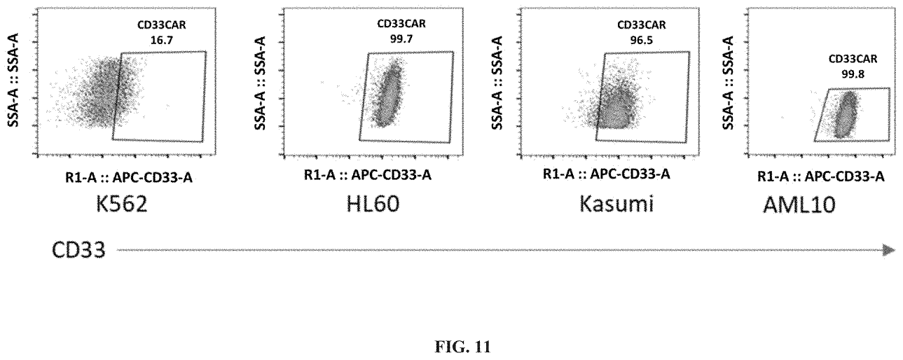

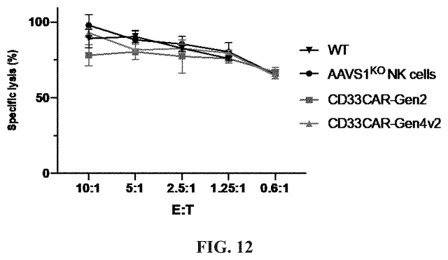

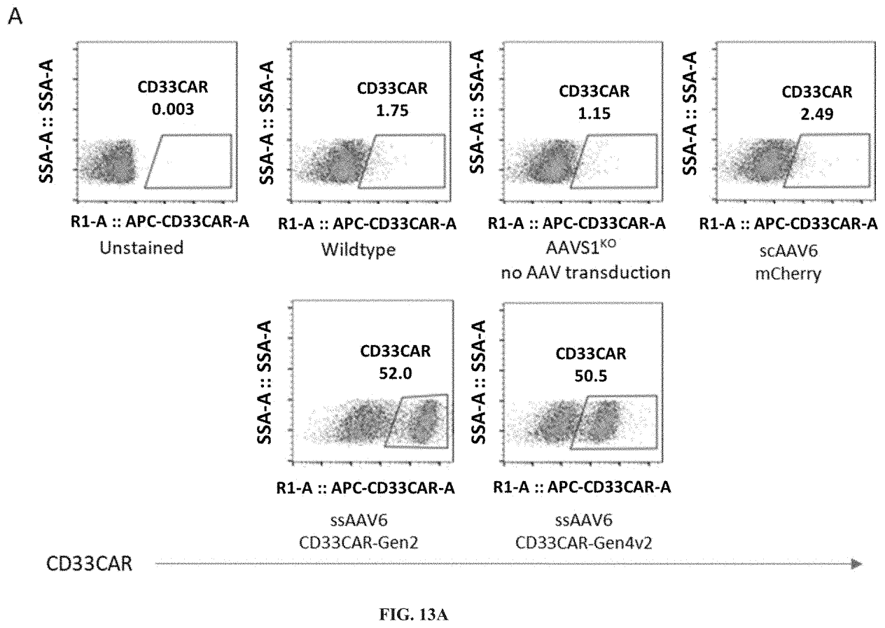

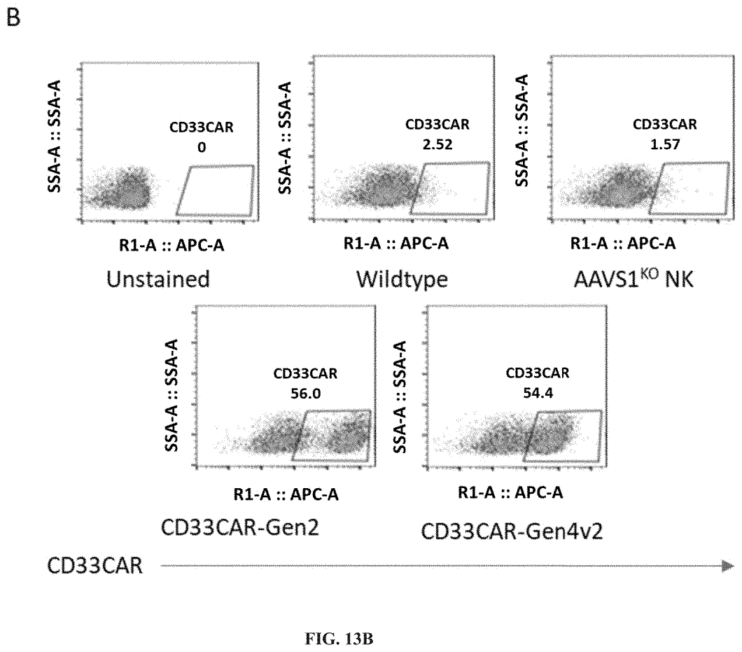

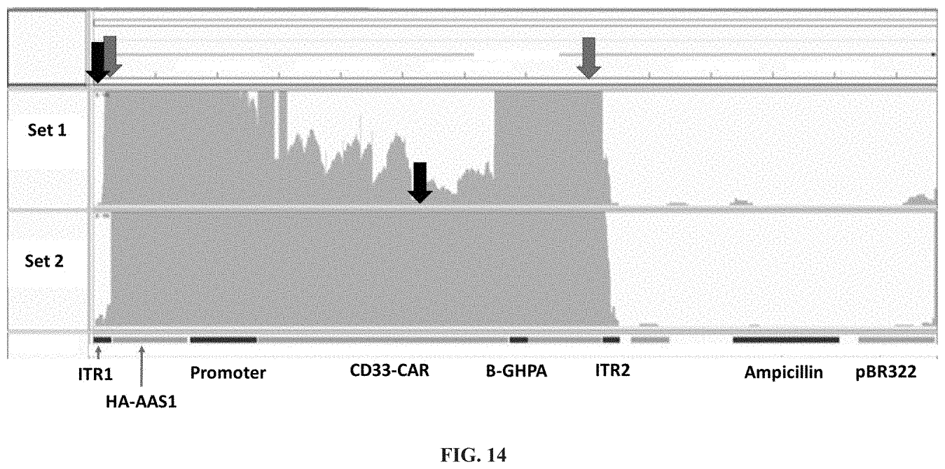

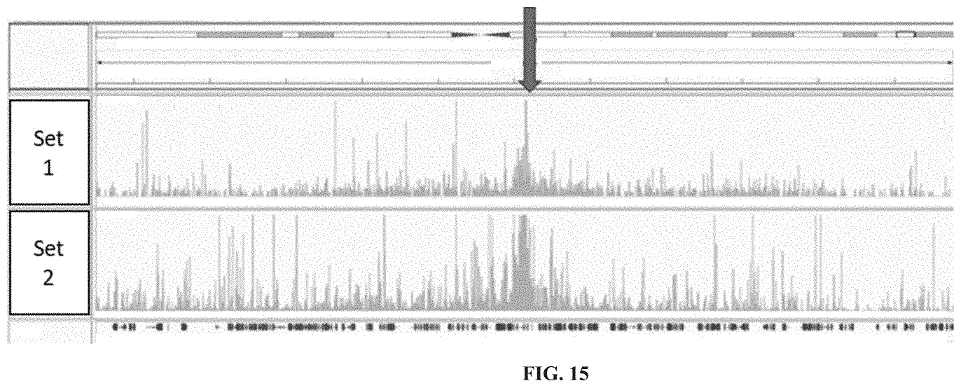

To study the cytotoxic effect of primary human CD33CAR NK cells against CD33 expressing AML cells, Calcein AM based cytotoxicity assay was performed. Two different CD33 expressing AML cell lines called Kasumi-1 and HL60 were used (FIG. 11) and cocultured them with NK cells isolated from peripheral blood collected of three different healthy individuals. CD33CAR-gen2 and CD33CAR-gen4v2 NK cells showed a significantly higher expression level of CD107a, an NK cell degranulation marker, when cocultured with Kasumi-1 or HL60 in comparison to wildtype or AAVS1^{K0} NK cells. This also resulted in a significantly higher specific lysis of Kasumi-1 by either CD33CAR NK cells. A higher killing ability of CD33CAR-Gen2 against HL60 was also observed (FIGS. 8A-8F). The specificity of enhanced tumor-killing of CD33CAR NK cells against CD33 expressing cancer cells was shown by performing cytotoxicity assay against K562 chronic myelogenous leukemia (CML) and did not see any improvement in killing ability of NK cells (FIG. 8I and FIG. 12). Importantly, significantly higher antitumor activity of CD33CAR NK cells was observed against AML-10, a primary human AML derived from a relapsed patient (FIGS. 8G and 8H, FIG. 12). Overall, CD33CAR-Gen2 NK showed better cytotoxicity in comparison to CD33CAR-Gen4v2 NK cells.

c) Discussion

Gene modification in primary human NK cells has always been challenging; reported herein is a successful, highly efficient site-directed gene integration into human primary NK cells using a combination of electroporation of Cas9/RNP and single-stranded or self-complementary AAV6 gene delivery through HR and homology-independent gene insertion (CRISPaint). Here for the first time, it is shown how the expression level of genes regulating HR and NHEJ pathways in human NK cells alter during expansion with FC21 and there is provided an optimal condition for site-directed gene insertion. It was also demonstrated that AAVS1 can host and express exogenous genes at a highly efficient level, as shown previously in T cells and NK cells. Furthermore, it was shown that a range of HAs from 30-1000 bp that can be used for gene insertion into the AAVS1 locus in NK cells, but that the shortest optimal length is at 300 bp when used in scAAV6. This helps researchers to choose an optimal HA based on the size of their exogenous DNA for introducing in NK cells. CRISPaint gene insertion can be used for tagging endogenous genes and be used for studying the biology of proteins in NK cells.

The combination of Cas9/RNP and AAV6 gene delivery was used and two different human primary CD33CAR NK cells were generated with enhanced anti-AML activity. These results also showed that the gene-modified NK cells can be subsequently expanded with FC21, enabling the production of large numbers of gene-modified NK cells for cancer immunotherapy. Overall, the method shown herein can be used for several applications in immunology, cancer immunotherapy, and studying the biology of NK cells.

TABLE 1

| | |
|---|---|
| Forward primer | 5′ TTCTCCTGTGGATTCGGGTCAC 3′ |
| | (SEQ ID NO: 34) |
| Reverse primer | 5′ CTCTCTGGCTCCATCGTAAGCA 3′ |
| | (SEQ ID NO: 35) |

TABLE 2

| Condition 1 | |
|---|---|
| Reverse-1200 bp | TCCTGGGCAAACAGCATAA |
| (1) | (SEQ ID NO: 36) |
| Forward-CD33CAR | GAGCTGCAGAAGGACAAGAT |
| (1) | (SEQ ID NO: 37) |
| Condition 2 | |
| Reverse-CD33CAR | CTCTGTGTCATCTGGATGTCTG |
| (2) | (SEQ ID NO: 38) |
| Forward-1200 bp | CTTTGAGCTCTACTGGCTTCTG |
| (2) | (SEQ ID NO: 39) |
| Condition 3 | |
| Reverse-1200 bp | TCCTGGGCAAACAGCATAA |
| (1) | (SEQ ID NO: 40) |
| Forward-1200 bp | CTTTGAGCTCTACTGGCTTCTG |
| (2) | (SEQ ID NO: 41) |

TABLE 3

| Primer | Name/View | | |
|---|---|---|---|
| set | point | Direction | Sequence |
| 1 | 600 bp HA | Reverse | GCGAGTGAAGACGGCATG |
| | AAVS1 | | (SEQ ID NO: 42) |
| | | Forward | GTCTGTGCTAGCTCTTCCAG |
| | | | (SEQ ID NO: 43) |
| 2 | CD33CAR- | Reverse | GCGATGTCAGAAGGGTAAA |
| | Gen2 | | (SEQ ID NO: 44) |
| | | Forward | GGCGGACACTCTGACTACAT |
| | | | (SEQ ID NO: 45) |

TLA was performed with 2 independent primer sets specific for the vector sequence (Table 3).

Sequence variants. Detected sequence variants are presented in table 4. The frequency of this variant might indicate a variation in the vector used.

TABLE 4
Identified sequence variants.

| | | | | Primer | | Primer | |
|---|---|---|---|---|---|---|---|
| | | Refer- | | set 1 | | set 2 | |
| Region | Position | ence | Mutation | Coverage | % | Coverage | % |
| 600bp | 4,116 | T | C | 193 | 37 | 341 | 14 |
| HA | | | | | | | |
| AAVS1 | | | | | | | |

Identifying Structural Variants

4 vector-vector breakpoints were found. All fusions were located at the annotated homology arm. Due to the heterogeneous nature of the sample it is expected that these fusions are only present in a subset of the sample. It should be noted that three out of four fusions show 9-12 bp homology which might indicate technical bias.

Vector: 149 (head) fused to Vector: 4116 (tail)
with 9 homologous bases
(SEQ ID NO: 46)
GGCATGGGGTTGGGTGAGGGAGGAGAGATGCCCGGAGAGGACCCAGA
CACGGGGAGGATCCGCTCAGAGGACATCACGTGGTGCAGCGGCGCGC
CGGCCGCAGAAAGGGAGTAGAGGCGGCCACGACCTGGTGAACACCTA
GGACGCACCATTCTCACAAAGGGAGTTTTCCACACGGACACCCCCCT
CCTCACCACAGCCCTGCCAGGACGGGGCTGGCTACTGGCCTTATCTC
Vector: 149 (head) fused to Vector: 4,113 (tail)
with 12 homologous bases
(SEQ ID NO: 47)
GCGAGTGAAGACGGCATGGGGTTGGGTGAGGGAGGAGAGATGCCCGG
AGAGGACCCAGACACGGGGAGGATCCGCTCAGAGGACATCACGTGGT
GCAGCGGCGCGCCGGCCGCAGGAAGGGAGTAGAGGCGGCCACGACCT
GGTGAACACCTAGGACGCACCATTCTCACAAAGGGAGTTTTCCACAC
GGACACCCCCCTCCTCACCACAGCCCTGCCAGGACGGGGCTGGCTAC
TGGCCTTA
Vector: 155 (head) fused to Vector: 4,163 (tail)
with 9 homologous bases
(SEQ ID NO: 48)
GCGAGTGAAGACGGCATGGGGTTGGGTGAGGGAGGAGAGATGCCCGG
AGAGGACCCAGACACGGGGAGGATCCGCTCAGAGGACATCACGTGGT
GCAGCGGCGCGCAGAGAGGGAGTGGCCAACTCCATCACTAGGGGTTC
CTGCGGCCGCAGAAAGGGAGTAGAGGCGGCCACGACCTGGTGAACAC
CTAGGACGCACCATTCTCACAAAGGGAGTTTTCCACACGGA
Vector: 158 (head) fused to Vector: 4,121 (tail)
with 4 homologous bases
(SEQ ID NO: 49)
GCGAGTGAAGACGGCATGGGGTTGGGTGAGGGAGGAGAGATGCCCGG
AGAGGACCCAGACACGGGGAGGATCCGCTCAGAGGACATCACGTGGT
GCAGCGGCCGCAGAAAGGGAGTAGAGGCGGCCACGACCTGGTGAACA
CCTAGGACGCACCATTCTCACAAAGGGAGTTTTCCACACGGACACCC
CCCTCCTCACCACAGCCCTGCCAGGACGGGGCTGGCTACTGGCCTT

### B. REFERENCES

Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat Methods 10, 1213-1218 (2013).
de Vree, P. J. et al. Targeted sequencing by proximity ligation for comprehensive variant detection and local haplotyping. Nat Biotechnol 32, 1019-1025 (2014).
Dutour, A. et al. In Vitro and In Vivo Antitumor Effect of Anti-CD33 Chimeric Receptor-Expressing EBV-CTL against CD33 Acute Myeloid Leukemia. Adv Hematol 2012, 683065 (2012).
Foust, K. D. et al. Therapeutic AAV9-mediated suppression of mutant SOD1 slows disease progression and extends survival in models of inherited ALS. Mol Ther 21, 2148-2159 (2013).
He, X. et al. Knock-in of large reporter genes in human cells via CRISPR/Cas9-induced homology-dependent and independent DNA repair. Nucleic Acids Res 44, e85 (2016).
Hsiau, T. et al. Inference of CRISPR Edits from Sanger Trace Data. bioRxiv, 251082 (2018).
Li, K., Wang, G., Andersen, T., Zhou, P. & Pu, W. T. Optimization of genome engineering approaches with the CRISPR/Cas9 system. PLoS One 9, e105779 (2014).
Li, Y., Hermanson, D. L., Moriarity, B. S. & Kaufman, D. S. Human iPSC-Derived Natural Killer Cells Engineered with Chimeric Antigen Receptors Enhance Anti-tumor Activity. Cell Stem Cell 23, 181-192 e185 (2018).
Liu, J., Zhou, G., Zhang, L. & Zhao, Q. Building Potent Chimeric Antigen Receptor T Cells With CRISPR Genome Editing. Front Immunol 10, 456 (2019).
MacLeod, D. T. et al. Integration of a CD19 CAR into the TCR Alpha Chain Locus Streamlines Production of Allogeneic Gene-Edited CAR T Cells. Mol Ther 25, 949-961 (2017).
Mali, P. et al. RNA-guided human genome engineering via Cas9. Science 339, 823-826 (2013).
McCarty, D. M. Self-complementary AAV vectors; advances and applications. Mol Ther 16, 1648-1656 (2008).
Mendell, J. R. et al. Single-Dose Gene-Replacement Therapy for Spinal Muscular Atrophy. N Engl J Med 377, 1713-1722 (2017).
Moseman, J. E., Foltz, J. A., Sorathia, K., Heipertz, E. L. & Lee, D. A. Evaluation of serum-free media formulations in feeder cell-stimulated expansion of natural killer cells. Cytotherapy (2020).
Naeimi Kararoudi, M. et al. CD38 deletion of human primary NK cells eliminates daratumumab-induced fratricide and boosts their effector activity. Blood (2020).
Naeimi Kararoudi, M. et al. Generation of Knock-out Primary and Expanded Human NK Cells Using Cas9 Ribonucleoproteins. J Vis Exp (2018).
Oceguera-Yanez, F. et al. Engineering the AAVS1 locus for consistent and scalable transgene expression in human iPSCs and their differentiated derivatives. Methods 101, 43-55 (2016).
Pomeroy, E. J. et al. A Genetically Engineered Primary Human Natural Killer Cell Platform for Cancer Immunotherapy. Mol Ther 28, 52-63 (2020).
Ran, F. A. et al. Genome engineering using the CRISPR-Cas9 system. Nat Protoc 8, 2281-2308 (2013).
Schmid-Burgk, J. L., Honing, K., Ebert, T. S. & Hornung, V. CRISPaint allows modular base-specific gene tagging using a ligase-4-dependent mechanism. Nat Commun 7, 12338 (2016).
Somanchi, S. S., Senyukov, V. V., Denman, C. J. & Lee, D. A. Expansion, purification, and functional assessment of human peripheral blood NK cells. J Vis Exp (2011).
Song, F. & Stieger, K. Optimizing the DNA Donor Template for Homology-Directed Repair of Double-Strand Breaks. Mol Ther Nucleic Acids 7, 53-60 (2017).
Suzuki, K. et al. In vivo genome editing via CRISPR/Cas9 mediated homology-independent targeted integration. Nature 540, 144-149 (2016).

C. SEQUENCES
SEQ ID NO: 1 30 bp right homology arm
gattggtgacagaaaagccccatccttagg
SEQ ID NO: 2 30 bp left homology arm
ttatctgtcccctccaccccacagtggggc
SEQ ID NO: 3 300 bp right homology arm
gattggtgacagaaaagccccatccttaggcctcctccacctagtctcctgatattgggtctaacccccacctcctgttaggcagattccttat
ctggtgacacacccccatttcctggagccatctctctccttgccagaacctctaaggtttgcttacgatggagccagagaggatcctgggag
ggagagcttggcagggggtgggagggaagggggggatgcgtgacctgcccggttctcagtggccaccctgcgctaccctctcccagaa
cctgagctgctctgacgcggctgtc
SEQ ID NO: 4 300 bp left homology arm
gttctcctgtggattcgggtcacctctcactcctttcatttgggcagctcccctaccccccttacctctctagtctgtgctagctcttccagccccc
tgtcatggcatcttccaggggtccgagagctcagctagtcttcttcctccaacccgggcccctatgtccacttcaggacagcatgtttgctgcc
tccagggatcctgtgtccccgagctgggaccaccttatattcccagggccggttaatgtggctctggttctgggtacttttatctgtcccctcca
ccccacagtggggc
SEQ ID NO: 5 500 bp right homology arm
gattggtgacagaaaagccccatccttaggcctcctccttcctagtctcctgatattgggtctaacccccacctcctgttaggcagattccttat
ctggtgacacacccccatttcctggagccatctctctccttgccagaacctctaaggtttgcttacgatggagccagagaggatcctgggag
ggagagcttggcagggggtgggagggaagggggggatgcgtgacctgcccggttctcagtggccaccctgcgctaccctctcccagaa
cctgagctgctctgacgcggctgtctggtgcgtttcactgatcctggtgctgcagcttccttacacttcccaagaggagaagcagtttggaaa
aacaaaatcagaataagttggtcctgagttctaactttggctcttcacctttctagtccccaatttatattgttcctccgtgcgtcagttttacctgtg
agataaggccagtagccagccccgtcctggcag
SEQ ID NO: 6 500 bp left homology arm
tcccttttccttctccttctggggcctgtgccatctctcgtttcttaggatggccttctccgacggatgtctcccttgcgtcccgcctccccttcttg
taggcctgcatcatcaccgtttttctggacaaccccaaagtaccccgtctccctggctttagccacctctccatcctcttgctttctttgcctgga
caccccgttctcctgtggattcgggtcacctctcactcctttcatttgggcagctcccctaccccccttacctctctagtctgtgctagctcttcca
gccccctgtcatggcatcttccaggggtccgagagctcagctagtcttcttcctccaacccgggcccctatgtccacttcaggacagcatgtt
tgctgcctccagggatcctgtgtccccgagctgggaccaccttatattcccagggccggttaatgtggctctggttctgggtacttttatctgtc
ccctccaccccacagtggggc
SEQ ID NO: 7 800 bp right homology arm
gattggtgacagaaaagccccatccttaggcctcctccttcctagtctcctgatattgggtctaacccccacctcctgttaggcagattccttat
ctggtgacacacccccatttcctggagccatctctctccttgccagaacctctaaggtttgcttacgatggagccagagaggatcctgggag
ggagagcttggcagggggtgggagggaagggggggatgcgtgacctgcccggttctcagtggccaccctgcgctaccctctcccagaa
cctgagctgctctgacgcggctgtctggtgcgtttcactgatcctggtgctgcagcttccttacacttcccaagaggagaagcagtttggaaa
aacaaaatcagaataagttggtcctgagttctaactttggctcttcacctttctagtccccaatttatattgttcctccgtgcgtcagttttacctgtg
agataaggccagtagccagccccgtcctggcagggctgtggtgaggaggggggtgtccgtgtggaaaactccctttgtgagaatggtgc
gtcctaggtgttcaccaggtcgtggccgcctctactccctttctctttctccatccttctttccttaaagagtccccagtgctatctgggacatattc
ctccgcccagagcagggtcccgcttccctaaggccctgctctgggcttctgggtttgagtccttggcaagcccaggagaggcgctcaggc
ttccctgtcccccttcctcgtccaccatctcatgcccctggctctcctgccccttccctacaggggttcctggctctgctcttcagactgagccc
cgttcccctgcatccccgttcccctgcatcccccttcccctgcatcccccagaggccccaggccacctacttggcctggaccccacgagag
gccaccccagccctgtctaccaggctgccttttgggtggattctcctccaactgtggggtgactgcttgg
SEQ ID NO: 8 800 bp left homology arm
tgctttctctgacctgcattctctcccctgggcctgtgccgctttctgtctgcagcttgtggcctgggtcacctctacggctggcccagatccttc
cctgccgcctccttcaggttccgtcttcctccactccctcttccccttgctctctgctgtgttgctgcccaaggatgctctttccggagcacttcct
tctcggcgctgcaccacgtgatgtcctctgagcggatcctccccgtgtctgggtcctctccgggcatctctcctccctcacccaaccccatgc
cgtcttcactcgctgggttcccttttccttctccttctggggcctgtgccatctctcgtttcttaggatggccttctccgacggatgtctcccttgcg
tcccgcctccccttcttgtaggcctgcatcatcaccgtttttctggacaaccccaaagtaccccgtctccctggctttagccacctctccatcct
cttgctttctttgcctggacaccccgttctcctgtggattcgggtcacctctcactcctttcatttgggcagctcccctaccccccttacctctcta
gtctgtgctagctcttccagccccctgtcatggcatcttccaggggtccgagagctcagctagtcttcttcctccaacccgggcccctatgtcc
acttcaggacagcatgtttgctgcctccagggatcctgtgtccccgagctgggaccaccttatattcccagggccggttaatgtggctctggt
tctgggtacttttatctgtcccctccaccccacagtggggc
SEQ ID NO: 9 PAMg (PAM + the sequence encoding crRNA)
Ccaatcctgtccctagtggcccc
SEQ ID NO: 10 splice acceptor
atcgatcgcaggcgcaatcttcgcatttcttttttccag
SEQ ID NO: 11 BGH polyA terminator
cctcgactgtgccttctagttgccagccatctgttgtttgcccctcccccgtgccttccttgaccctggaaggtgccactcccactgtcctttcct
aataaaatgaggaaattgcatcgcattgtctgagtaggtgtcattctattc
SEQ ID NO: 12 mCherry
gtgagcaagggcgaggaggataacatggccatcatcaaggagttcatgcgcttcaaggtgcacatggagggctccgtgaacggccacg
agttcgagatcgagggcgagggcgagggccgcccctacgagggcacccagaccgccaagctgaaggtgaccaagggtggccccctg
cccttcgcctgggacatcctgtcccctcagttcatgtacggctccaaggcctacgtgaagcaccccgccgacatccccgactacttgaagct
gtccttccccgagggcttcaagtgggagcgcgtgatgaacttcgaggacggcggcgtggtgaccgtgacccaggactcctccctgcagg
acggcgagttcatctacaaggtgaagctgcgcggcaccaacttcccctccgacggccccgtaatgcagaagaagaccatgggctggga
ggcctcctccgagcggatgtaccccgaggacggcgccctgaagggcgagatcaagcagaggctgaagctgaaggacggcggccact
acgacgctgaggtcaagaccacctacaaggccaagaagcccgtgcagctgcccggcgcctacaacgtcaacatcaagttggacatcac
ctcccacaacgaggactacaccatcgtggaacagtacgaacgcgccgagggccgccactccaccggcggcatggacgagctgtacaa
gtaa
SEQ ID NO: 13 30 bp plasmid with incorporated mCherry transgene.
ttatctgtcccctccaccccacagtggggccactagggacagcgatcgggtacatcgatcgcaggcgcaatcttcgcatttcttttttccaggt
gagcaagggcgaggaggataacatggccatcatcaaggagttcatgcgcttcaaggtgcacatggagggctccgtgaacggccacgag
ttcgagatcgagggcgagggcgagggccgcccctacgagggcacccagaccgccaagctgaaggtgaccaagggtggccccctgcc
cttcgcctgggacatcctgtcccctcagttcatgtacggctccaaggcctacgtgaagcaccccgccgacatccccgactacttgaagctgt
ccttccccgagggcttcaagtgggagcgcgtgatgaacttcgaggacggcggcgtggtgaccgtgacccaggactcctccctgcaggac
ggcgagttcatctacaaggtgaagctgcgcggcaccaacttcccctccgacggccccgtaatgcagaagaagaccatgggctgggagg
cctcctccgagcggatgtaccccgaggacggcgccctgaagggcgagatcaagcagaggctgaagctgaaggacggcggccactac
gacgctgaggtcaagaccacctacaaggccaagaagcccgtgcagctgcccggcgcctacaacgtcaacatcaagttggacatcacctc
ccacaacgaggactacaccatcgtggaacagtacgaacgcgccgagggccgccactccaccggcggcatggacgagctgtacaagta
acgcggccgccctcgactgtgccttctagttgccagccatctgttgtttgcccctcccccgtgccttccttgaccctggaaggtgccactccc
actgtcctttcctaataaaatgaggaaattgcatcgcattgtctgagtaggtgtcattctattcgattggtgacagaaaagccccatccttagg
SEQ ID NO: 14 300 bp plasmid with incorporated mCherry transgene.
gttctcctgtggattcgggtcacctctcactcctttcatttgggcagctcccctaccccccttacctctctagtctgtgctagctcttccagccccc
tgtcatggcatcttccaggggtccgagagctcagctagtcttcttcctccaacccgggcccctatgtccacttcaggacagcatgtttgctgcc
tccagggatcctgtgtccccgagctgggaccaccttatattcccagggccggttaatgtggctctggttctgggtacttttatctgtcccctcca
ccccacagtggggccactagggacagcgatcgggtacatcgatcgcaggcgcaatcttcgcatttcttttttccaggtgagcaagggcgag
gaggataacatggccatcatcaaggagttcatgcgcttcaaggtgcacatggagggctccgtgaacggccacgagttcgagatcgaggg
cgagggcgagggccgcccctacgagggcacccagaccgccaagctgaaggtgaccaagggtggccccctgcccttcgcctgggacat
cctgtcccctcagttcatgtacggctccaaggcctacgtgaagcaccccgccgacatccccgactacttgaagctgtccttccccgagggct
tcaagtgggagcgcgtgatgaacttcgaggacggcggcgtggtgaccgtgacccaggactcctccctgcaggacggcgagttcatctac
aaggtgaagctgcgcggcaccaacttcccctccgacggccccgtaatgcagaagaagaccatgggctgggaggcctcctccgagcgga
tgtaccccgaggacggcgccctgaagggcgagatcaagcagaggctgaagctgaaggacggcggccactacgacgctgaggtcaag
accacctacaaggccaagaagcccgtgcagctgcccggcgcctacaacgtcaacatcaagttggacatcacctcccacaacgaggacta
caccatcgtggaacagtacgaacgcgccgagggccgccactccaccggcggcatggacgagctgtacaagtaacgcggccgccctcg
actgtgccttctagttgccagccatctgttgtttgcccctcccccgtgccttccttgaccctggaaggtgccactcccactgtcctttcctaataa
aatgaggaaattgcatcgcattgtctgagtaggtgtcattctattcgattggtgacagaaaagccccatccttaggcctcctccttcctagtctc
ctgatattgggtctaacccccacctcctgttaggcagattccttatctggtgacacacccccatttcctggagccatctctctccttgccagaac
ctctaaggtttgcttacgatggagccagagaggatcctgggagggagagcttggcagggggtgggagggaagggggggatgcgtgac
ctgcccggttctcagtggccaccctgcgctaccctctcccagaacctgagctgctctgacgcggctgtc
SEQ ID NO: 15 500 bp plasmid with incorporated mCherry transgene.
tcccttttccttctccttctggggcctgtgccatctctcgtttcttaggatggccttctccgacggatgtctcccttgcgtcccgcctccccttcttg
taggcctgcatcatcaccgtttttctggacaaccccaaagtaccccgtctccctggctttagccacctctccatcctcttgctttctttgcctgga
caccccgttctcctgtggattcgggtcacctctcactcctttcatttgggcagctcccctaccccccttacctctctagtctgtgctagctcttcca
gccccctgtcatggcatcttccaggggtccgagagctcagctagtcttcttcctccaacccgggcccctatgtccacttcaggacagcatgtt
tgctgcctccagggatcctgtgtccccgagctgggaccaccttatattcccagggccggttaatgtggctctggttctgggtacttttatctgtc
ccctccaccccacagtggggccactagggacagcgatcgggtacatcgatcgcaggcgcaatcttcgcatttcttttttccaggtgagcaa
gggcgaggaggataacatggccatcatcaaggagttcatgcgcttcaaggtgcacatggagggctccgtgaacggccacgagttcgaga
tcgagggcgagggcgagggccgcccctacgagggcacccagaccgccaagctgaaggtgaccaagggtggccccctgcccttcgcc
tgggacatcctgtcccctcagttcatgtacggctccaaggcctacgtgaagcaccccgccgacatccccgactacttgaagctgtccttccc
cgagggcttcaagtgggagcgcgtgatgaacttcgaggacggcggcgtggtgaccgtgacccaggactcctccctgcaggacggcga
gttcatctacaaggtgaagctgcgcggcaccaacttcccctccgacggccccgtaatgcagaagaagaccatgggctgggaggcctcct
ccgagcggatgtaccccgaggacggcgccctgaagggcgagatcaagcagaggctgaagctgaaggacggcggccactacgacgct
gaggtcaagaccacctacaaggccaagaagcccgtgcagctgcccggcgcctacaacgtcaacatcaagaggacatcacctcccacaa
cgaggactacaccatcgtggaacagtacgaacgcgccgagggccgccactccaccggcggcatggacgagctgtacaagtaacgcgg
ccgccctcgactgtgccttctagttgccagccatctgttgtttgcccctcccccgtgccttccttgaccctggaaggtgccactcccactgtcct
ttcctaataaaatgaggaaattgcatcgcattgtctgagtaggtgtcattctattcgattggtgacagaaaagccccatccttaggcctcctcctt
cctagtctcctgatattgggtctaacccccacctcctgttaggcagattccttatctggtgacacacccccatttcctggagccatctctctcctt
gccagaacctctaaggtttgcttacgatggagccagagaggatcctgggagggagagcttggcagggggtgggagggaaggggggga
tgcgtgacctgcccggttctcagtggccaccctgcgctaccctctcccagaacctgagctgctctgacgcggctgtctggtgcgtttcactg
atcctggtgctgcagcttccttacacttcccaagaggagaagcagtttggaaaaacaaaatcagaataagttggtcctgagttctaactttggc
tcttcacctttctagtccccaatttatattgttcctccgtgcgtcagttttacctgtgagataaggccagtagccagccccgtcctggcag
SEQ ID NO: 16 800 bp plasmid with incorporated mCherry transgene.
tgctttctctgacctgcattctctcccctgggcctgtgccgctttctgtctgcagcttgtggcctgggtcacctctacggctggcccagatccttc
cctgccgcctccttcaggttccgtcttcctccactccctcttccccttgctctctgctgtgttgctgcccaaggatgctctttccggagcacttcct
tctcggcgctgcaccacgtgatgtcctctgagcggatcctccccgtgtctgggtcctctccgggcatctctcctccctcacccaaccccatgc
cgtcttcactcgctgggttcccttttccttctccttctggggcctgtgccatctctcgtttcttaggatggccttctccgacggatgtctcccttgcg
tcccgcctccccttcttgtaggcctgcatcatcaccgtttttctggacaaccccaaagtaccccgtctccctggctttagccacctctccatcct
cttgctttctttgcctggacaccccgttctcctgtggattcgggtcacctctcactcctttcatttgggcagctcccctaccccccttacctctcta
gtctgtgctagctcttccagccccctgtcatggcatcttccaggggtccgagagctcagctagtcttcttcctccaacccgggcccctatgtcc
acttcaggacagcatgtttgctgcctccagggatcctgtgtccccgagctgggaccaccttatattcccagggccggttaatgtggctctggt
tctgggtacttttatctgtcccctccaccccacagtggggccactagggacagcgatcgggtacatcgatcgcaggcgcaatcttcgcatttc
ttttttccaggtgagcaagggcgaggaggataacatggccatcatcaaggagttcatgcgcttcaaggtgcacatggagggctccgtgaac
ggccacgagttcgagatcgagggcgagggcgagggccgcccctacgagggcacccagaccgccaagctgaaggtgaccaagggtg
gccccctgcccttcgcctgggacatcctgtcccctcagttcatgtacggctccaaggcctacgtgaagcaccccgccgacatccccgacta
cttgaagctgtccttccccgagggcttcaagtgggagcgcgtgatgaacttcgaggacggcggcgtggtgaccgtgacccaggactcctc
cctgcaggacggcgagttcatctacaaggtgaagctgcgcggcaccaacttcccctccgacggccccgtaatgcagaagaagaccatgg
gctgggaggcctcctccgagcggatgtaccccgaggacggcgccctgaagggcgagatcaagcagaggctgaagctgaaggacggc
ggccactacgacgctgaggtcaagaccacctacaaggccaagaagcccgtgcagctgcccggcgcctacaacgtcaacatcaagttgg
acatcacctcccacaacgaggactacaccatcgtggaacagtacgaacgcgccgagggccgccactccaccggcggcatggacgagc
tgtacaagtaacgcggccgccctcgactgtgccttctagttgccagccatctgttgtttgcccctcccccgtgccttccttgaccctggaaggt
gccactcccactgtcctttcctaataaaatgaggaaattgcatcgcattgtctgagtaggtgtcattctattcgattggtgacagaaaagcccca
tccttaggcctcctccttcctagtctcctgatattgggtctaacccccacctcctgttaggcagattccttatctggtgacacacccccatttcctg
gagccatctctctccttgccagaacctctaaggtttgcttacgatggagccagagaggatcctgggagggagagcttggcagggggtggg
agggaagggggggatgcgtgacctgcccggttctcagtggccaccctgcgctaccctctcccagaacctgagctgctctgacgcggctgt
ctggtgcgtttcactgatcctggtgctgcagcttccttacacttcccaagaggagaagcagtttggaaaaacaaaatcagaataagttggtcct
gagttctaactttggctcttcacctttctagtccccaatttatattgttcctccgtgcgtcagttttacctgtgagataaggccagtagccagcccc
gtcctggcagggctgtggtgaggaggggggtgtccgtgtggaaaactccctttgtgagaatggtgcgtcctaggtgttcaccaggtcgtgg
ccgcctctactccctttctctttctccatccttctttccttaaagagtccccagtgctatctgggacatattcctccgcccagagcagggtcccgc
ttccctaaggccctgctctgggcttctgggtttgagtccttggcaagcccaggagaggcgctcaggcttccctgtcccccttcctcgtccacc
atctcatgcccctggctctcctgccccttccctacaggggttcctggctctgctcttcagactgagccccgttcccctgcatccccgttcccct
gcatcccccttcccctgcatcccccagaggccccaggccacctacttggcctggaccccacgagaggccaccccagccctgtctaccag
gctgccttttgggtggattctcctccaactgtggggtgactgcttgg
SEQ ID NO: 17 (crRNA)
GGGGCCACTAGGGACAGGAT
SEQ ID NO: 18 scFV:
atgctgctgctggtgacctctctgctgctgtgcgagctgccacacccagccttcctgctgatcccagacatccagatgacacagagcccca
gctccctgagcgcctccgtgggcgacagagtgaccatcacatgtagggcctctgagagcgtggataactatggcatcagcttcatgaattg
gtttcagcagaagcctggcggcgccccaaagctgctgatctacgcagccagcatgcagggctccggcgtgccctctcggttctccggctc
tggcagcggcaccgacttcaccctgacaatctctagcctgcagccagacgatttcgccacatactattgccagcagagcaaggaggtgcc
ctggacctttggccagggcacaaaggtggagatcaagggctccacctctggcagcggcaagcctggcagcggagagggctccacaaa
gggacaggtgcagctggtgcagtccggagccgaggtgaagaagccaggctcctctgtgaaggtgtcttgtaaggccagcggctatacctt
cacagactacaacatgcactgggtgcgccaggcaccaggacagggcctggagtggatcggctacatctatccttacaacggcggcaccg
gctataatcagaagtttaagtccaaggccaccatcacagccgatgagtctaccaatacagcctacatggagctgagcagcctgcggtccga
ggacacagccgtgtactattgcgcccggggcagacccgctatggactattggggccagggcaccctggtgacagtgtctag
SEQ ID NO: 19 IgG4-Hinge:
gagagcaagtacggaccaccttgcccaccatgtcctgcaccagagttcctgggaggaccttccgtgttcctgtttcctccaaagccaaagg
acaccctgatgatcagccggaccccagaggtgacatgcgtggtggtggacgtgagccaggaggaccccgaggtgcagttcaactggta
cgtggatggcgtggaggtgcacaatgccaagaccaagccaagagaggagcagtttaactccacctatagggtggtgtctgtgctgacagt
gctgcaccaggactggctgaacggcaaggagtacaagtgcaaggtgtccaataagggcctgccttcctctatcgagaagaccatctctaa
ggcaaagggacagccaagggagccacaggtgtatacactgccccctagccaggaggagatgaccaagaaccaggtgtccctgacatgt
ctggtgaagggcttttacccttctgacatcgccgtggagtgggagagcaatggccagccagagaacaattataagaccacaccacccgtg
ctggactctgatggcagcttctttctgtacagccgcctgaccgtggataagtcccggtggcaggagggcaacgtgttctcctgctctgtgatg
cacgaggccctgcacaatcactacacacagaagagcctgtccctgtctctgggcaag
SEQ ID NO: 20 CD28:
Atgttttgggtgctggtggtggtgggaggcgtgctggcctgttattccctgctggtgaccgtggccttcatcatcttttgggtgcgctccaagc
ggagccggggcggacactctgactacatgaacatgaccccacggagacccggacctacaaggaagcactatcagccctacgcccctcc
acgggacttcgcagcatatcgcagc
SEQ ID NO: 21 CD3z:
Cgggtgaagtttagcagatccgccgatgcaccagcatatcagcagggacagaatcagctgtacaacgagctgaatctgggcaggcgcg
aggagtacgacgtgctggataagaggcggggccgggaccccgagatgggaggcaagcccaggcgcaagaaccctcaggagggcct
gtataatgagctgcagaaggacaagatggccgaggcctacagcgagatcggcatgaagggagagcggagaaggggcaagggacacg
atggcctgtatcagggcctgtccaccgccacaaaggacacctacgatgcactgcacatgcaggccctgccacctcggtga
SEQ ID NO: 22 CD33CAR-Gen2-Cloned in ssAAV BackBone (FIG. 16):
cctgcaggcagctgcgcgctcgctcgctcactgaggccgcccgggcaaagcccgggcgtcgggcgacctttggtcgcccggcctcagt
gagcgagcgagcgcgcagagagggagtggccaactccatcactaggggttcctgcggccggcgcgccgctgcaccacgtgatgtcct
ctgagcggatcctccccgtgtctgggtcctctccgggcatctctcctccctcacccaaccccatgccgtcttcactcgctgggttcccttttcct
tctccttctggggcctgtgccatctctcgtttcttaggatggccttctccgacggatgtctcccttgcgtcccgcctccccttcttgtaggcctgc
atcatcaccgtttttctggacaaccccaaagtaccccgtctccctggctttagccacctctccatcctcttgctttctttgcctggacaccccgttc
tcctgtggattcgggtcacctctcactcctttcatttgggcagctcccctaccccccttacctctctagtctgtgctagctcttccagccccctgtc
atggcatcttccaggggtccgagagctcagctagtcttcttcctccaacccgggcccctatgtccacttcaggacagcatgtttgctgcctcc
agggatcctgtgtccccgagctgggaccaccttatattcccagggccggttaatgtggctctggttctgggtacttttatctgtcccctccacc
ccacagtggggccactagggacagcgatcgggtacatcgatcacgagactagcctcgagaagcttgatatcgaattccacggggttgga
cgcgtcttaattaaggatccaaggtcaggaacagagaaacaggagaatatgggccaaacaggatatctgtggtaagcagttcctgccccg
gctcagggccaagaacagttggaacagcagaatatgggccaaacaggatatctgtggtaagcagttcctgccccggctcagggccaaga
acagatggtccccagatgcggtcccgccctcagcagtttctagagaaccatcagatgtttccagggtgccccaaggacctgaaatgaccct
gtgccttatttgaactaaccaatcagttcgcttctcgcttctgttcgcgcgcttctgctccccgagctctatataagcagagctcgtttagtgaac
cgtcagatcgcctggagacgccatccacgctgttttgacctccatagaagacaccgactctagaggatcgatcccccgggctgcaggaatt
caagcgagaagacaagggcagaaagcaccgccaccatgctgctgctggtgacctctctgctgctgtgcgagctgccacacccagccttc
ctgctgatcccagacatccagatgacacagagccccagctccctgagcgcctccgtgggcgacagagtgaccatcacatgtagggcctct
gagagcgtggataactatggcatcagcttcatgaattggtttcagcagaagcctggcggcgccccaaagctgctgatctacgcagccagc
atgcagggctccggcgtgccctctcggttctccggctctggcagcggcaccgacttcaccctgacaatctctagcctgcagccagacgatt
tcgccacatactattgccagcagagcaaggaggtgccctggacctttggccagggcacaaaggtggagatcaagggctccacctctggc
agcggcaagcctggcagcggagagggctccacaaagggacaggtgcagctggtgcagtccggagccgaggtgaagaagccaggctc
ctctgtgaaggtgtcttgtaaggccagcggctataccttcacagactacaacatgcactgggtgcgccaggcaccaggacagggcctgga
gtggatcggctacatctatccttacaacggcggcaccggctataatcagaagtttaagtccaaggccaccatcacagccgatgagtctacca
atacagcctacatggagctgagcagcctgcggtccgaggacacagccgtgtactattgcgcccggggcagacccgctatggactattgg
ggccagggcaccctggtgacagtgtctagcgagagcaagtacggaccaccttgcccaccatgtcctgcaccagagttcctgggaggacc
ttccgtgttcctgtttcctccaaagccaaaggacaccctgatgatcagccggaccccagaggtgacatgcgtggtggtggacgtgagccag
gaggaccccgaggtgcagttcaactggtacgtggatggcgtggaggtgcacaatgccaagaccaagccaagagaggagcagtttaact
ccacctatagggtggtgtctgtgctgacagtgctgcaccaggactggctgaacggcaaggagtacaagtgcaaggtgtccaataagggcc
tgccttcctctatcgagaagaccatctctaaggcaaagggacagccaagggagccacaggtgtatacactgccccctagccaggaggag
atgaccaagaaccaggtgtccctgacatgtctggtgaagggcttttacccttctgacatcgccgtggagtgggagagcaatggccagccag
agaacaattataagaccacaccacccgtgctggactctgatggcagcttctttctgtacagccgcctgaccgtggataagtcccggtggcag
gagggcaacgtgttctcctgctctgtgatgcacgaggccctgcacaatcactacacacagaagagcctgtccctgtctctgggcaagatgtt
ttgggtgctggtggtggtgggaggcgtgctggcctgttattccctgctggtgaccgtggccttcatcatcttttgggtgcgctccaagcggag
ccggggcggacactctgactacatgaacatgaccccacggagacccggacctacaaggaagcactatcagccctacgcccctccacgg
gacttcgcagcatatcgcagccgggtgaagtttagcagatccgccgatgcaccagcatatcagcagggacagaatcagctgtacaacga
gctgaatctgggcaggcgcgaggagtacgacgtgctggataagaggcggggccgggaccccgagatgggaggcaagcccaggcgc
aagaaccctcaggagggcctgtataatgagctgcagaaggacaagatggccgaggcctacagcgagatcggcatgaagggagagcgg
agaaggggcaagggacacgatggcctgtatcagggcctgtccaccgccacaaaggacacctacgatgcactgcacatgcaggccctgc
cacctcggtgaaagtaacgccctcgactgtgccttctagttgccagccatctgttgtttgcccctcccccgtgccttccttgaccctggaaggt
gccactcccactgtcctttcctaataaaatgaggaaattgcatcgcattgtctgagtaggtgtcattctattcgattggtgacagaaaagcccca
tccttaggcctcctccttcctagtctcctgatattgggtctaacccccacctcctgttaggcagattccttatctggtgacacacccccatttcctg
gagccatctctctccttgccagaacctctaaggtttgcttacgatggagccagagaggatcctgggagggagagcttggcagggggtggg
agggaagggggggatgcgtgacctgcccggttctcagtggccaccctgcgctaccctctcccagaacctgagctgctctgacgcggctgt
ctggtgcgtttcactgatcctggtgctgcagcttccttacacttcccaagaggagaagcagtttggaaaaacaaaatcagaataagttggtcct
gagttctaactttggctcttcacctttctagtccccaatttatattgttcctccgtgcgtcagttttacctgtgagataaggccagtagccagcccc
gtcctggcagggctgtggtgaggaggggggtgtccgtgtggaaaactccctttgtgagaatggtgcgtcctaggtgttcaccaggtcgtgg
ccgcctctactccctttctgcggccgcaggaacccctagtgatggagttggccactccctctctgcgcgctcgctcgctcactgaggccgg
gcgaccaaaggtcgcccgacgcccgggctttgcccgggcggcctcagtgagcgagcgagcgcgcagctgcctgcaggggcgcctga
tgcggtattttctccttacgcatctgtgcggtatttcacaccgcatacgtcaaagcaaccatagtacgcgccctgtagcggcgcattaagcgc
ggcgggtgtggtggttacgcgcagcgtgaccgctacacttgccagcgccctagcgcccgctcctttcgctttcttcccttcctttctcgccac
gttcgccggctttccccgtcaagctctaaatcgggggctccctttagggttccgatttagtgctttacggcacctcgaccccaaaaaacttgat
ttgggtgatggttcacgtagtgggccatcgccctgatagacggtttttcgccctttgacgttggagtccacgttctttaatagtggactcttgttc
caaactggaacaacactcaaccctatctcgggctattcttttgatttataagggattttgccgatttcggcctattggttaaaaaatgagctgattt
aacaaaaatttaacgcgaattttaacaaaatattaacgtttacaattttatggtgcactctcagtacaatctgctctgatgccgcatagttaagcca
gccccgacacccgccaacacccgctgacgcgccctgacgggcttgtctgctcccggcatccgcttacagacaagctgtgaccgtctccg
ggagctgcatgtgtcagaggttttcaccgtcatcaccgaaacgcgcgagacgaaagggcctcgtgatacgcctatttttataggttaatgtca
tgataataatggtttcttagacgtcaggtggcacttttcggggaaatgtgcgcggaacccctatttgtttatttttctaaatacattcaaatatgtat
ccgctcatgagacaataaccctgataaatgcttcaataatattgaaaaaggaagagtatgagtattcaacatttccgtgtcgcccttattcccttt
tttgcggcattttgccttcctgtttttgctcacccagaaacgctggtgaaagtaaaagatgctgaagatcagttgggtgcacgagtgggttacat
cgaactggatctcaacagcggtaagatccttgagagttttcgccccgaagaacgttttccaatgatgagcacttttaaagttctgctatgtggc
gcggtattatcccgtattgacgccgggcaagagcaactcggtcgccgcatacactattctcagaatgacttggttgagtactcaccagtcac
agaaaagcatcttacggatggcatgacagtaagagaattatgcagtgctgccataaccatgagtgataacactgcggccaacttacttctga
caacgatcggaggaccgaaggagctaaccgcttttttgcacaacatgggggatcatgtaactcgccttgatcgttgggaaccggagctgaa
tgaagccataccaaacgacgagcgtgacaccacgatgcctgtagcaatggcaacaacgttgcgcaaactattaactggcgaactacttact
ctagcttcccggcaacaattaatagactggatggaggcggataaagttgcaggaccacttctgcgctcggcccttccggctggctggtttatt
gctgataaatctggagccggtgagcgtgggtctcgcggtatcattgcagcactggggccagatggtaagccctcccgtatcgtagttatcta
cacgacggggagtcaggcaactatggatgaacgaaatagacagatcgctgagataggtgcctcactgattaagcattggtaactgtcaga
ccaagtttactcatatatactttagattgatttaaaacttcatttttaatttaaaaggatctaggtgaagatcctttttgataatctcatgaccaaaatc
ccttaacgtgagttttcgttccactgagcgtcagaccccgtagaaaagatcaaaggatcttcttgagatcctttttttctgcgcgtaatctgctgc
ttgcaaacaaaaaaaccaccgctaccagcggtggtttgtttgccggatcaagagctaccaactctttttccgaaggtaactggcttcagcaga
gcgcagataccaaatactgtccttctagtgtagccgtagttaggccaccacttcaagaactctgtagcaccgcctacatacctcgctctgcta
atcctgttaccagtggctgctgccagtggcgataagtcgtgtcttaccgggttggactcaagacgatagttaccggataaggcgcagcggtc
gggctgaacggggggttcgtgcacacagcccagcttggagcgaacgacctacaccgaactgagatacctacagcgtgagctatgagaa
agcgccacgcttcccgaagggagaaaggcggacaggtatccggtaagcggcagggtcggaacaggagagcgcacgagggagcttcc
agggggaaacgcctggtatctttatagtcctgtcgggtttcgccacctctgacttgagcgtcgatttttgtgatgctcgtcaggggggcggag
cctatggaaaaacgccagcaacgcggcctttttacggttcctggccttttgctggccttttgctcacatgt
The ITR1 sequence corresponds to nucleic acid position 1-141 of SEQ ID NO: 22;
the MND-CD33CAR-gen2 construct corresponds to nucleic acid position 156-4118 of SEQ ID NO: 22;
the left 600 bp homology arm AAVS1 corresponds to nucleic acid position 156-759 of SEQ ID NO: 22;
the MND promoter corresponds to nucleic acid position 783-1322 of SEQ ID NO: 22;
the sequence encoding CD33 CAR gen2 corresponds to nucleic acid position 1329-3362 of SEQ ID NO: 22;
the sequence encoding scFV-CD33 corresponds to nucleic acid position 1329-2128 of SEQ ID NO: 22;
the sequence encoding IgG-hingeCD4 corresponds to nucleic acid position 2130-2816 of SEQ ID NO: 22;
the sequence encoding CD28 corresponds to nucleic acid position 2814-3023 of SEQ ID NO: 22;
the sequence encoding CD3zeta corresponds to nucleic acid position 3024-3362 of SEQ ID NO: 22;
the BGHPA corresponds to nucleic acid position 3372-3518 of SEQ ID NO: 22;
the BGH poly corresponds to nucleic acid position 3378-3489 of SEQ ID NO: 22;
the right 600 bp homology arm AAVS1 corresponds to nucleic acid position 3519-4118 of SEQ ID NO: 22;
ITR2 sequence corresponds to nucleic acid position 4127-4267 of SEQ ID NO: 22.
SEQ ID NO: 23 CD33CAR-Gen4v2 (FIG. 17):
cctgcaggcagctgcgcgctcgctcgctcactgaggccgcccgggcaaagcccgggcgtcgggcgacctttggtcgcccggcctcagt
gagcgagcgagcgcgcagagagggagtggccaactccatcactaggggttcctgcggccggcgcgccgctgcaccacgtgatgtcct
ctgagcggatcctccccgtgtctgggtcctctccgggcatctctcctccctcacccaaccccatgccgtcttcactcgctgggttcccttttcct
tctccttctggggcctgtgccatctctcgtttcttaggatggccttctccgacggatgtctcccttgcgtcccgcctccccttcttgtaggcctgc
atcatcaccgtttttctggacaaccccaaagtaccccgtctccctggctttagccacctctccatcctcttgctttctttgcctggacaccccgttc
tcctgtggattcgggtcacctctcactcctttcatttgggcagctcccctaccccccttacctctctagtctgtgctagctcttccagccccctgtc
atggcatcttccaggggtccgagagctcagctagtcttcttcctccaacccgggcccctatgtccacttcaggacagcatgtttgctgcctcc
agggatcctgtgtccccgagctgggaccaccttatattcccagggccggttaatgtggctctggttctgggtacttttatctgtcccctccacc
ccacagtggggccactagggacagcgatcgggtacatcgatcacgagactagcctcgagaagcttgatatcgaattccacggggttgga
cgcgtcttaattaaggatccaaggtcaggaacagagaaacaggagaatatgggccaaacaggatatctgtggtaagcagttcctgccccg
gctcagggccaagaacagttggaacagcagaatatgggccaaacaggatatctgtggtaagcagttcctgccccggctcagggccaaga
acagatggtccccagatgcggtcccgccctcagcagtttctagagaaccatcagatgtttccagggtgccccaaggacctgaaatgaccct
gtgccttatttgaactaaccaatcagttcgcttctcgcttctgttcgcgcgcttctgctccccgagctctatataagcagagctcgtttagtgaac
cgtcagatcgcctggagacgccatccacgctgttttgacctccatagaagacaccgactctagaggatcgatcccccgggctgcaggaatt
caagcgagaagacaagggcagaaagcaccgccaccatgctgctgctggtgacctccctgctgctgtgcgagctgccacaccctgcctttc
tgctgatcccagacatccagatgacacagagccccagctccctgtctgccagcgtgggcgacagagtgaccatcacatgtagggcctccg
agtctgtggataactatggcatcagctttatgaattggttccagcagaagccaggaggcgcccctaagctgctgatctacgcagcctccatg
cagggctctggcgtgcccagccgctttagcggctccggctctggcaccgatttcaccctgacaatctctagcctgcagccagacgattttgc
cacatactattgccagcagtccaaggaggtgccctggaccttcggccagggcacaaaggtggagatcaagggcagcacctccggctctg
gcaagcctggctccggagagggctctacaaagggacaggtgcagctggtgcagagcggagccgaggtgaagaagccaggctcctctg
tgaaggtgagctgtaaggcctccggctatacctttacagactacaacatgcactgggtgagacaggcaccaggacagggcctggagtgg
atcggctacatctatccttacaacggcggcaccggctataatcagaagttcaagagcaaggccaccatcacagccgatgagtccaccaata
cagcctacatggagctgagcagcctgaggagcgaggacacagccgtgtactattgcgccagaggcaggcctgctatggactattggggc
cagggcaccctggtgacagtgtctagcgagtccaagtacggaccaccttgcccaccatgtccagcaccagagtttctgggaggacctagc
gtgtttctgttccctccaaagccaaaggacaccctgatgatcagcagaacccccgaggtgacatgcgtggtggtggacgtgtcccaggag
gaccccgaggtgcagtttaactggtacgtggatggcgtggaggtgcacaatgccaagaccaagcctagagaggagcagttcaactccac
ctatagggtggtgtctgtgctgacagtgctgcaccaggactggctgaacggcaaggagtacaagtgcaaggtgtctaataagggcctgcc
atcctctatcgagaagaccatcagcaaggccaagggccagcctagggagccacaggtgtatacactgcccccttcccaggaggagatga
ccaagaaccaggtgtctctgacatgtctggtgaagggcttctacccatccgacatcgccgtggagtgggagtctaatggccagcccgaga
acaattataagaccacaccacccgtgctggactctgatggcagcttctttctgtactctcgcctgaccgtggataagagccggtggcaggag
ggcaacgtgtttagctgctccgtgatgcacgaggccctgcacaatcactacacacagaagtctctgagcctgtccctgggcaagagcaac
ctgttcgtggcctcctggatcgccgtgatgatcatctttcgcatcggcatggccgtggccatcttctgctgtttctttttcccatccggaggctct
ggaggaggctccggctggcggagaaagcggaaggagaagcagagcgagacctcccctaaggagtttctgacaatctatgaggacgtg
aaggatctgaagaccaggcgcaatcacgagcaggagcagaccttcccaggaggaggctctacaatctacagcatgatccagtcccaga
gcagcgccccaaccagccaggagccagcctatacactgtactctctgatccagcctagccggaagtctggcagccgcaagcggaacca
ctccccatctttcaattctaccatctatgaagtgatcggcaagagccagcctaaggcccagaacccagccagactgtccaggaaggagctg
gagaattttgacgtgtactctggaggcagcggaggaggctctggccgcgtgaagttcagccggtccgccgatgccccagcctataagca
gggccagaaccagctgtacaacgagctgaatctgggccggagagaggagtacgacgtgctggataagaggcggggccgggaccccg
agatgggaggcaagccccggagaaagaaccctcaggagggcctgtataatgagctgcagaaggacaagatggccgaggcctactccg
agatcggcatgaagggagagaggcgccggggcaagggacacgatggcctgtatcagggcctgagcaccgccacaaaggacacctac
gatgccctgcacatgcaggccctgcctccacggtgatgaaagtaacgccctcgactgtgccttctagttgccagccatctgttgtttgcccct
cccccgtgccttccttgaccctggaaggtgccactcccactgtcctttcctaataaaatgaggaaattgcatcgcattgtctgagtaggtgtcat
tctattcgattggtgacagaaaagccccatccttaggcctcctccttcctagtctcctgatattgggtctaacccccacctcctgttaggcagatt
ccttatctggtgacacacccccatttcctggagccatctctctccttgccagaacctctaaggtttgcttacgatggagccagagaggatcctg
ggagggagagcttggcagggggtgggagggaagggggggatgcgtgacctgcccggttctcagtggccaccctgcgctaccctctcc
cagaacctgagctgctctgacgcggctgtctggtgcgtttcactgatcctggtgctgcagcttccttacacttcccaagaggagaagcagttt
ggaaaaacaaaatcagaataagttggtcctgagttctaactttggctcttcacctttctagtccccaatttatattgttcctccgtgcgtcagtttta
cctgtgagataaggccagtagccagccccgtcctggcagggctgtggtgaggaggggggtgtccgtgtggaaaactccctttgtgagaat
ggtgcgtcctaggtgttcaccaggtcgtggccgcctctactccctttctgcggccgcaggaacccctagtgatggagttggccactccctct
ctgcgcgctcgctcgctcactgaggccgggcgaccaaaggtcgcccgacgcccgggctttgcccgggcggcctcagtgagcgagcga
gcgcgcagctgcctgcaggggcgcctgatgcggtattttctccttacgcatctgtgcggtatttcacaccgcatacgtcaaagcaaccatagt
acgcgccctgtagcggcgcattaagcgcggcgggtgtggtggttacgcgcagcgtgaccgctacacttgccagcgccctagcgcccgct
cctttcgctttcttcccttcctttctcgccacgttcgccggctttccccgtcaagctctaaatcgggggctccctttagggttccgatttagtgcttt
acggcacctcgaccccaaaaaacttgatttgggtgatggttcacgtagtgggccatcgccctgatagacggtttttcgccctttgacgttgga
gtccacgttctttaatagtggactcttgttccaaactggaacaacactcaaccctatctcgggctattcttttgatttataagggattttgccgattt
cggcctattggttaaaaaatgagctgatttaacaaaaatttaacgcgaattttaacaaaatattaacgtttacaattttatggtgcactctcagtac
aatctgctctgatgccgcatagttaagccagccccgacacccgccaacacccgctgacgcgccctgacgggcttgtctgctcccggcatc
cgcttacagacaagctgtgaccgtctccgggagctgcatgtgtcagaggttttcaccgtcatcaccgaaacgcgcgagacgaaagggcct
cgtgatacgcctatttttataggttaatgtcatgataataatggtttcttagacgtcaggtggcacttttcggggaaatgtgcgcggaacccctat
ttgtttatttttctaaatacattcaaatatgtatccgctcatgagacaataaccctgataaatgcttcaataatattgaaaaaggaagagtatgagta
ttcaacatttccgtgtcgcccttattcccttttttgcggcattttgccttcctgtttttgctcacccagaaacgctggtgaaagtaaaagatgctgaa
gatcagttgggtgcacgagtgggttacatcgaactggatctcaacagcggtaagatccttgagagttttcgccccgaagaacgttttccaatg
atgagcacttttaaagttctgctatgtggcgcggtattatcccgtattgacgccgggcaagagcaactcggtcgccgcatacactattctcag
aatgacttggttgagtactcaccagtcacagaaaagcatcttacggatggcatgacagtaagagaattatgcagtgctgccataaccatgag
tgataacactgcggccaacttacttctgacaacgatcggaggaccgaaggagctaaccgcttttttgcacaacatgggggatcatgtaactc
gccttgatcgttgggaaccggagctgaatgaagccataccaaacgacgagcgtgacaccacgatgcctgtagcaatggcaacaacgttg
cgcaaactattaactggcgaactacttactctagcttcccggcaacaattaatagactggatggaggcggataaagttgcaggaccacttctg
cgctcggcccttccggctggctggtttattgctgataaatctggagccggtgagcgtgggtctcgcggtatcattgcagcactggggccaga
tggtaagccctcccgtatcgtagttatctacacgacggggagtcaggcaactatggatgaacgaaatagacagatcgctgagataggtgcc
tcactgattaagcattggtaactgtcagaccaagtttactcatatatactttagattgatttaaaacttcatttttaatttaaaaggatctaggtgaag
atcctttttgataatctcatgaccaaaatcccttaacgtgagttttcgttccactgagcgtcagaccccgtagaaaagatcaaaggatcttcttga
gatcctttttttctgcgcgtaatctgctgcttgcaaacaaaaaaaccaccgctaccagcggtggtttgtttgccggatcaagagctaccaactct
ttttccgaaggtaactggcttcagcagagcgcagataccaaatactgtccttctagtgtagccgtagttaggccaccacttcaagaactctgta
gcaccgcctacatacctcgctctgctaatcctgttaccagtggctgctgccagtggcgataagtcgtgtcttaccgggttggactcaagacga
tagttaccggataaggcgcagcggtcgggctgaacggggggttcgtgcacacagcccagcttggagcgaacgacctacaccgaactga
gatacctacagcgtgagctatgagaaagcgccacgcttcccgaagggagaaaggcggacaggtatccggtaagcggcagggtcggaa
caggag agcgcacgagggagcttccagggggaaacgcctggtatctttatagtcctgtcgggtttcgccacctctgacttgagcgtcgattt
ttgtgatgctcgtcaggggggcggagcctatggaaaaacgccagcaacgcggcctttttacggttcctggccttttgctggccttttgctcac
atgt
The ITR1 sequence corresponds to nucleic acid position 1-141 of SEQ ID NO: 23; the MND-
CD33CAR-gen2 construct corresponds to nucleic acid position 156-4415 of SEQ ID NO: 23;
the left 600 bp homology arm AAVS1 corresponds to nucleic acid position 156-759 of SEQ ID
NO: 23; the MND promoter corresponds to nucleic acid position 783-1322 of SEQ ID NO: 23;
the sequence encoding CD33 CAR gen2 corresponds to nucleic acid position 1329-3659 of SEQ
ID NO: 23; the sequence encoding scFV-CD33 corresponds to nucleic acid position 1329-2129
of SEQ ID NO: 23; the sequence encoding IgG-hingeCD4 corresponds to nucleic acid position
2130-2816 of SEQ ID NO: 23; the sequence encoding NKG2D TM corresponds to nucleic acid
position 2817-2909 of SEQ ID NO: 23; the sequence encoding 2B4 corresponds to nucleic acid
position 2934-3293 of SEQ ID NO: 23; the sequence encoding CD3zeta corresponds to nucleic
acid position 3318-3659; the BGHPA corresponds to nucleic acid position 3669-3815 of SEQ
ID NO: 23; the BGH poly corresponds to nucleic acid position 3675-3786 of SEQ ID NO: 23;
the right 600 bp homology arm AAVS1 corresponds to nucleic acid position 3816-4415 of SEQ
ID NO: 23; ITR2 sequence corresponds to nucleic acid position 4424-4564 of SEQ ID NO: 23.
SEQ ID NO: 24 NKG2D Transmembrane domain:
Agcaacctgttcgtggcctcctggatcgccgtgatgatcatctttcgcatcggcatggccgtggccatcttctgctgtttctttttcccatcc
SEQ ID NO: 25 Linker:
Ggaggctctggaggaggctccggc
SEQ ID NO: 26 2B4:
Tggcggagaaagcggaaggagaagcagagcgagacctcccctaaggagtttctgacaatctatgaggacgtgaaggatctgaagacc
aggcgcaatcacgagcaggagcagaccttcccaggaggaggctctacaatctacagcatgatccagtcccagagcagcgccccaacca
gccaggagccagcctatacactgtactctctgatccagcctagccggaagtctggcagccgcaagcggaaccactccccatctttcaattct
accatctatgaagtgatcggcaagagccagcctaaggcccagaacccagccagactgtccaggaaggagctggagaattttgacgtgta
ctct
SEQ ID NO: 27 Linker:
Ggaggcagcggaggaggctctggc
SEQ ID NO: 28 CD3z:
Cgcgtgaagttcagccggtccgccgatgccccagcctataagcagggccagaaccagctgtacaacgagctgaatctgggccggaga
gaggagtacgacgtgctggataagaggcggggccgggaccccgagatgggaggcaagccccggagaaagaaccctcaggagggcc
tgtataatgagctgcagaaggacaagatggccgaggcctactccgagatcggcatgaagggagagaggcgccggggcaagggacac
gatggcctgtatcagggcctgagcaccgccacaaaggacacctacgatgccctgcacatgcaggccctgcctccacggtgatga
SEQ ID NO: 29, anti-CD33 ScFv.
atgctgctgctggtgacctccctgctgctgtgcgagctgccacaccctgcctttctgctgatcccagacatccagatgacacagagccccag
ctccctgtctgccagcgtgggcgacagagtgaccatcacatgtagggcctccgagtctgtggataactatggcatcagctttatgaattggtt
ccagcagaagccaggaggcgcccctaagctgctgatctacgcagcctccatgcagggctctggcgtgcccagccgctttagcggctccg
gctctggcaccgatttcaccctgacaatctctagcctgcagccagacgattttgccacatactattgccagcagtccaaggaggtgccctgg
accttcggccagggcacaaaggtggagatcaagggcagcacctccggctctggcaagcctggctccggagagggctctacaaaggga
caggtgcagctggtgcagagcggagccgaggtgaagaagccaggctcctctgtgaaggtgagctgtaaggcctccggctatacctttac
agactacaacatgcactgggtgagacaggcaccaggacagggcctggagtggatcggctacatctatccttacaacggcggcaccggct
ataatcagaagttcaagagcaaggccaccatcacagccgatgagtccaccaatacagcctacatggagctgagcagcctgaggagcgag
gacacagccgtgtactattgcgccagaggcaggcctgctatggactattggggccagggcaccctggtgacagtgtctagc
SEQ ID NO: 30, MND promoter
atcgatcacgagactagcctcgagaagcttgatatcgaattccacggggttggacgcgtcttaattaaggatccaaggtcaggaacagaga
aacaggagaatatgggccaaacaggatatctgtggtaagcagttcctgccccggctcagggccaagaacagttggaacagcagaatatg
ggccaaacaggatatctgtggtaagcagttcctgccccggctcagggccaagaacagatggtccccagatgcggtcccgccctcagcag
tttctagagaaccatcagatgtttccagggtgccccaaggacctgaaatgaccctgtgccttatttgaactaaccaatcagttcgcttctcgctt
ctgttcgcgcgcttctgctccccgagctctatataagcagagctcgtttagtgaaccgtcagatcgcctggagacgccatccacgctgttttg
acctccatagaagacaccgactctagaggatcgatcccccgggctgcaggaattcaagcgagaagacaagggcagaaagcacc
SEQ ID NO: 31, 600 bp, LHA, AAVS1 (gen4v2 and gen2)
gctgcaccacgtgatgtcctctgagcggatcctccccgtgtctgggtcctctccgggcatctctcctccctcacccaaccccatgccgtcttc
actcgctgggttcccttttccttctccttctggggcctgtgccatctctcgtttcttaggatggccttctccgacggatgtctcccttgcgtcccgc
ctccccttcttgtaggcctgcatcatcaccgtttttctggacaaccccaaagtaccccgtctccctggctttagccacctctccatcctcttgcttt
ctttgcctggacaccccgttctcctgtggattcgggtcacctctcactcctttcatttgggcagctcccctaccccccttacctctctagtctgtg
ctagctcttccagccccctgtcatggcatcttccaggggtccgagagctcagctagtcttcttcctccaacccgggcccctatgtccacttcag
gacagcatgtttgctgcctccagggatcctgtgtccccgagctgggaccaccttatattcccagggccggttaatgtggctctggttctgggt
acttttatctgtcccctccaccccacagtggggc
SEQ ID NO: 32, 600 bp, RHA, AAVS1 (gen4v2 and gen2)
Gattggtgacagaaaagccccatccttaggcctcctccttcctagtctcctgatattgggtctaacccccacctcctgttaggcagattccttat
ctggtgacacacccccatttcctggagccatctctctccttgccagaacctctaaggtttgcttacgatggagccagagaggatcctgggag
ggagagcttggcagggggtgggagggaagggggggatgcgtgacctgcccggttctcagtggccaccctgcgctaccctctcccagaa
cctgagctgctctgacgcggctgtctggtgcgtttcactgatcctggtgctgcagcttccttacacttcccaagaggagaagcagtttggaaa
aacaaaatcagaataagttggtcctgagttctaactttggctcttcacctttctagtccccaatttatattgttcctccgtgcgtcagttttacctgtg
agataaggccagtagccagccccgtcctggcagggctgtggtgaggaggggggtgtccgtgtggaaaactccctttgtgagaatggtgc
gtcctaggtgttcaccaggtcgtggccgcctctactccctttct
SEQ ID NO: 33, gRNA sequence that targets AAVS1
GGGGCCACTAGGGACAGGAT
SEQ ID NO: 34.
TTCTCCTGTGGATTCGGGTCAC
SEQ ID NO: 35.
CTCTCTGGCTCCATCGTAAGCA
SEQ ID NO: 36.
TCCTGGGCAAACAGCATAA
SEQ ID NO: 37.
GAGCTGCAGAAGGACAAGAT
SEQ ID NO: 38.
CTCTGTGTCATCTGGATGTCTG
SEQ ID NO: 39.
CTTTGAGCTCTACTGGCTTCTG
SEQ ID NO: 40.
TCCTGGGCAAACAGCATAA
SEQ ID NO: 41.
CTTTGAGCTCTACTGGCTTCTG
SEQ ID NO: 42.
GCGAGTGAAGACGGCATG
SEQ ID NO: 43.
GTCTGTGCTAGCTCTTCCAG
SEQ ID NO: 44.
GCGATGTCAGAAGGGTAAA
SEQ ID NO: 45.
GGCGGACACTCTGACTACAT
SEQ ID NO: 46
GGCATGGGGTTGGGTGAGGGAGGAGAGATGCCCGGAGAGGACCCAGACACGGGGA
GGATCCGCTCAGAGGACATCACGTGGTGCAGCGGCGCGCCGGCCGCAGAAAGGGA
GTAGAGGCGGCCACGACCTGGTGAACACCTAGGACGCACCATTCTCACAAAGGGAG
TTTTCCACACGGACACCCCCCTCCTCACCACAGCCCTGCCAGGACGGGGCTGGCTAC
TGGCCTTATCTC
SEQ ID NO: 47
GCGAGTGAAGACGGCATGGGGTTGGGTGAGGGAGGAGAGATGCCCGGAGAGGACC
CAGACACGGGGAGGATCCGCTCAGAGGACATCACGTGGTGCAGCGGCGCGCCGGC
CGCAGGAAGGGAGTAGAGGCGGCCACGACCTGGTGAACACCTAGGACGCACCATT
CTCACAAAGGGAGTTTTCCACACGGACACCCCCCTCCTCACCACAGCCCTGCCAGG
ACGGGGCTGGCTACTGGCCTTA
SEQ ID NO: 48
GCGAGTGAAGACGGCATGGGGTTGGGTGAGGGAGGAGAGATGCCCGGAGAGGACC
CAGACACGGGGAGGATCCGCTCAGAGGACATCACGTGGTGCAGCGGCGCGCAGAG
AGGGAGTGGCCAACTCCATCACTAGGGGTTCCTGCGGCCGCAGAAAGGGAGTAGAG
GCGGCCACGACCTGGTGAACACCTAGGACGCACCATTCTCACAAAGGGAGTTTTCC
ACACGGA
SEQ ID NO: 49
GCGAGTGAAGACGGCATGGGGTTGGGTGAGGGAGGAGAGATGCCCGGAGAGGACC
CAGACACGGGGAGGATCCGCTCAGAGGACATCACGTGGTGCAGCGGCCGCAGAAA
GGGAGTAGAGGCGGCCACGACCTGGTGAACACCTAGGACGCACCATTCTCACAAAG
GGAGTTTTCCACACGGACACCCCCCTCCTCACCACAGCCCTGCCAGGACGGGGCTG
GCTACTGGCCTT
SEQ ID NO: 50 (PAMgPAMg mCherry construct, FIG. 22)
CCAATCCTGTCCCTAGTGGCCCCCACTAGGGACAGCGATCGGGTACATCGATCGCA
GGCGCAATCTTCGCATTTCTTTTTTCCAGGTGAGCAAGGGCGAGGAGGATAACATG
GCCATCATCAAGGAGTTCATGCGCTTCAAGGTGCACATGGAGGGCTCCGTGAACGG
CCACGAGTTCGAGATCGAGGGCGAGGGCGAGGGCCGCCCCTACGAGGGCACCCAG
ACCGCCAAGCTGAAGGTGACCAAGGGTGGCCCCCTGCCCTTCGCCTGGGACATCCT
GTCCCCTCAGTTCATGTACGGCTCCAAGGCCTACGTGAAGCACCCCGCCGACATCCC
CGACTACTTGAAGCTGTCCTTCCCCGAGGGCTTCAAGTGGGAGCGCGTGATGAACTT
CGAGGACGGCGGCGTGGTGACCGTGACCCAGGACTCCTCCCTGCAGGACGGCGAGT
TCATCTACAAGGTGAAGCTGCGCGGCACCAACTTCCCCTCCGACGGCCCCGTAATGC
AGAAGAAGACCATGGGCTGGGAGGCCTCCTCCGAGCGGATGTACCCCGAGGACGG
CGCCCTGAAGGGCGAGATCAAGCAGAGGCTGAAGCTGAAGGACGGCGGCCACTAC
GACGCTGAGGTCAAGACCACCTACAAGGCCAAGAAGCCCGTGCAGCTGCCCGGCGC
CTACAACGTCAACATCAAGTTGGACATCACCTCCCACAACGAGGACTACACCATCG
TGGAACAGTACGAACGCGCCGAGGGCCGCCACTCCACCGGCGGCATGGACGAGCTG
TACAAGTAACGCGGCCGCCCTCGACTGTGCCTTCTAGTTGCCAGCCATCTGTTGTTT
GCCCCTCCCCCGTGCCTTCCTTGACCCTGGAAGGTGCCACTCCCACTGTCCTTTCCTA
ATAAAATGAGGAAATTGCATCGCATTGTCTGAGTAGGTGTCATTCTATTCCCAATCC
TGTCCCTAGTGGCCCC
The first PAM sequence corresponds to nucleic acid position 1-3 of SEQ ID NO: 50; the first
sequence encoding crRNA corresponds to nucleic acid position 4-23 of SEQ ID NO: 50; the
splice acceptor sequence corresponds to nucleic acid position 47-85 of SEQ ID NO: 50;
mCherry codon (optimized) corresponds to nucleic acid position 86-793 of SEQ ID NO: 50; the
BGHpA sequence corresponds to nucleic acid position 803-949 of SEQ ID NO: 50; the second
PAM sequence corresponds to nucleic acid position 950-952 of SEQ ID NO: 50; the second
sequence encoding crRNA corresponds to nucleic acid position 953-972 of SEQ ID NO: 50.
SEQ ID NO: 51 (PAMgRNA mCherry construct sequence, FIG. 21)
CCAATCCTGTCCCTAGTGGCCCCCACTAGGGACAGCGATCGGGTACATCGATCGCA
GGCGCAATCTTCGCATTTCTTTTTTCCAGGTGAGCAAGGGCGAGGAGGATAACATG
GCCATCATCAAGGAGTTCATGCGCTTCAAGGTGCACATGGAGGGCTCCGTGAACGG
CCACGAGTTCGAGATCGAGGGCGAGGGCGAGGGCCGCCCCTACGAGGGCACCCAG
ACCGCCAAGCTGAAGGTGACCAAGGGTGGCCCCCTGCCCTTCGCCTGGGACATCCT
GTCCCCTCAGTTCATGTACGGCTCCAAGGCCTACGTGAAGCACCCCGCCGACATCCC
CGACTACTTGAAGCTGTCCTTCCCCGAGGGCTTCAAGTGGGAGCGCGTGATGAACTT
CGAGGACGGCGGCGTGGTGACCGTGACCCAGGACTCCTCCCTGCAGGACGGCGAGT
TCATCTACAAGGTGAAGCTGCGCGGCACCAACTTCCCCTCCGACGGCCCCGTAATGC
AGAAGAAGACCATGGGCTGGGAGGCCTCCTCCGAGCGGATGTACCCCGAGGACGG
CGCCCTGAAGGGCGAGATCAAGCAGAGGCTGAAGCTGAAGGACGGCGGCCACTAC
GACGCTGAGGTCAAGACCACCTACAAGGCCAAGAAGCCCGTGCAGCTGCCCGGCGC
CTACAACGTCAACATCAAGTTGGACATCACCTCCCACAACGAGGACTACACCATCG
TGGAACAGTACGAACGCGCCGAGGGCCGCCACTCCACCGGCGGCATGGACGAGCTG
TACAAGTAACGCGGCCGCCCTCGACTGTGCCTTCTAGTTGCCAGCCATCTGTTGTTT
GCCCCTCCCCCGTGCCTTCCTTGACCCTGGAAGGTGCCACTCCCACTGTCCTTTCCTA
ATAAAATGAGGAAATTGCATCGCATTGTCTGAGTAGGTGTCATTCTATTC
The PAM sequence corresponds to nucleic acid position 1-3 of SEQ ID NO: 51;
the sequence encoding crRNA corresponds to nucleic acid position 4-23 of SEQ ID NO: 51;
the splice acceptor corresponds to nucleic acid position 47-85 of SEQ ID NO: 51;
the mCherry codon (optimized) corresponds to nucleic acid position 86-793 of SEQ ID NO: 51;
the BGHpA sequence corresponds to nucleic acid position 803-949 of SEQ ID NO: 51.
SEQ ID NO: 52.
Cccctccaccccacagtggggccactagggacaggattggtgacagaaaagccccatccttaggc
SEQ ID NO: 53
Cccctccaccccacagtggggccactagggacag
SEQ ID NO: 54
Attggtgacagaaaagccccatccttaggc
SEQ ID NO: 55
Cccctccaccccacagtggggccactaggga
SEQ ID NO: 56
Cccctccaccccac
SEQ ID NO: 57
Cccctccaccccacagtggggccac
SEQ ID NO: 58
gattggtgacagaaaagccccatccttaggc

1. A plasmid for use with clustered regularly interspaced short palindromic repeat (CRISPR)/CRISPR-associated 9 (Cas9) integration systems wherein the plasmid comprises in order a left homology arm, a polynucleotide sequence encoding a chimeric antigen receptor (CAR) polypeptide, and a right homology arm; wherein the left and right homology arms are each 1000 bp in length or less.

2. The plasmid of claim 1, wherein the CAR polypeptide comprises a transmembrane domain, a co-stimulatory domain, a CD3ζ signaling domain, and a single-chain variable fragment (scFV) that specifically binds to a receptor on a target cell, wherein the receptor comprises CD33, wherein the transmembrane domain of the CAR polypeptide comprises a CD4 transmembrane domain, a CD8 transmembrane domain, a CD28 transmembrane domain, a CD3ζ transmembrane domain, or an NKG2D transmembrane domain, and wherein the co-stimulatory domain of the CAR polypeptide comprises a 2B4 domain, a CD28 co-stimulatory domain, a 4-1 BB co-stimulatory domain, or any combination thereof.

3-15. (canceled)

16. An Adeno-associated viral (AAV) vector comprising the plasmid of claim 1.

17-21. (canceled)

22. A modified cell comprising the AAV vector of claim 16, wherein the modified cell is a natural killer (NK) cell or NK T cell.

23-24. (canceled)

25. A method of treating a cancer in a subject comprising administering to a subject with a cancer the modified cell of claim 22.

26-50. (canceled)

51. A method of creating a chimeric antigen receptor (CAR) natural killer (NK) cell or a CAR NK T cell comprising
a) obtaining a ribonucleoprotein (RNP) complex comprising a class 2 CRISPR/Cas endonuclease (Cas9) complexed with a corresponding CRISPR/Cas guide RNA and an AAV vector comprising a plasmid comprising a polynucleotide sequence encoding a chimeric antigen receptor (CAR) polypeptide; wherein the polynucleotide sequence is flanked by homology arms; and wherein the homology arms are 1000 bp in length or less; and
b) introducing the polynucleotide sequence encoding the CAR polypeptide and the RNP complex into an NK cell or an NK T cell; wherein the polynucleotide sequence encoding the CAR polypeptide is introduced into the NK cell or NK T cell via infection with the AAV into the NK cell or NK T cell; wherein the RNP complex hybridizes to a target sequence within the genomic DNA of the NK cell or NK T cell and the DNA repair enzymes of the NK cell or NK T cell insert the polynucleotide sequence encoding the CAR polypeptide into the host genome at the target sequence within the genomic DNA of the cell thereby creating a CAR NK cell or CAR NK T cell.

52. The method of claim 51, wherein the NK cells or NK T cells are primary or expanded NK cells or NK T cells.

53. The method of claim 52, wherein the primary NK cells or NK T cells are incubated for about 4 to 10 days in the presence of IL-2 prior to infection or wherein the primary NK cells or NK T cells are expanded for about 4 to 10 days in the presence of irradiated feeder cells, plasma membrane particles, or exosomes prior to infection.

54. (canceled)

55. The method of claim 53, wherein the irradiated feeder cells, plasma membrane particles, or exosomes express membrane bound 4-1BBL, membrane-bound IL-21, or membrane-bound IL-15 or any combination thereof.

56. The method of claim 51, further comprising expanding the CAR NK cell or CAR NK T cell with IL-2 or with irradiated feeder cells, plasma membrane particles, or exosomes following infection, wherein the irradiated feeder cells, plasma membrane particles, or exosomes express membrane bound 4-1BBL, membrane-bound IL-21, or membrane-bound IL-15 or any combination thereof.

57. (canceled)

58. The method of claim 51, wherein the NK cell or NK T cell is infected with about 5 to 500K MOI of the AAV.

59. The method of claim 51, wherein the RNP complex is introduced into the NK cell or NK T cell via electroporation.

60. The method of claim 51, wherein the RNP complex is introduced into the NK cell or NK T cell via transfection; and wherein the RNP complex is encoded on the same or a different AAV.

61. The method of claim 51, wherein the CAR polypeptide comprises a transmembrane domain, a co-stimulatory domain, a CD3ζ signaling domain, and a single-chain variable fragment (scFV) that specifically binds to a receptor on a target cell.

62. The method of claim 61, wherein the receptor comprises CD33.

63. (canceled)

64. The method of claim 61, wherein the transmembrane domain of the CAR polypeptide comprises a CD4 transmembrane domain, a CD8 transmembrane domain, a CD28 transmembrane domain, a CD3ζ transmembrane domain, or a NKG2D transmembrane domain and/or wherein the co-stimulatory domain of the CAR polypeptide comprises a 2B4 domain, a CD28 co-stimulatory domain, a 4-1 BB co-stimulatory domain, or any combination thereof.

65. (canceled)

66. (canceled)

67. The method of claim 51, wherein the homology arms are each 600 bp in length.

68. (canceled)

69. The method of claim 51, wherein the homology arms specifically hybridize to the Adeno-Associated Virus Integration Site 1 (AAVS1) of chromosome 19 of humans.

70. The method of claim 51, wherein the plasmid further comprises a murine leukemia virus-derived (MND) promoter.

71. The method of claim 51, wherein the serotype of the AAV comprises AAV6.

72. The method of claim 51, wherein the vector is a single stranded AAV (ssAAV) or a self-complimentary AAV (scAAV).

73. The method of claim 51, wherein the vector comprises a sequence at least 90% identical to SEQ ID NO: 22 or SEQ ID NO: 23 or a fragment thereof.

74-115. (canceled)

116. A method of genetically modifying a natural killer (NK) cell or NK T cell comprising
a) obtaining a ribonucleoprotein (RNP) complex comprising a class 2 CRISPR/Cas endonuclease (Cas9) complexed with a corresponding CRISPR/Cas guide RNA and an AAV vector comprising a plasmid comprising a polynucleotide sequence encoding a chimeric antigen receptor (CAR); wherein the polynucleotide sequence is adjacent to one PAM and one polynucleotide sequence encoding crRNA or flanked by two PAMs and two polynucleotide sequences encoding crRNAs; and
b) introducing the polynucleotide sequence encoding the CAR polypeptide and the RNP complex into the NK cell or NK T cell; wherein the polynucleotide sequence encoding the CAR polypeptide is introduced into the cell via infection with the Adeno-associated virus (AAV) into a target cell; wherein in the ribonucleoprotein (RNP) complex hybridizes to a target sequence within the genomic DNA of the cell, and the cell's DNA repair enzymes insert the polynucleotide sequence encoding the chimeric antigen receptor (CAR) into the host genome at the target sequence, thereby creating a modified cell.

117. The method of claim 116, wherein the plasmid comprises in order one PAM sequence and one polynucleotide sequence encoding crRNAs, the polynucleotide sequence encoding the CAR polypeptide, and one PAM sequence and one polynucleotide sequence encoding crRNA.


SEQUENCE LISTING (58 sequences)

SEQ ID NO: 1 - DNA, 30 bp, Artificial Sequence
  Synthetic Sequence
gattggtgacagaaaagccccatccttagg

SEQ ID NO: 2 - DNA, 30 bp, Artificial Sequence
  Synthetic Sequence
ttatctgtcccctccaccccacagtggggc

SEQ ID NO: 3 - DNA, 300 bp, Artificial Sequence
  Synthetic Sequence
gattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtc

SEQ ID NO: 4 - DNA, 300 bp, Artificial Sequence
  Synthetic Sequence
gttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggc

SEQ ID NO: 5 - DNA, 500 bp, Artificial Sequence
  Synthetic Sequence
gattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcag

SEQ ID NO: 6 - DNA, 500 bp, Artificial Sequence
  Synthetic Sequence
tcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggc

SEQ ID NO: 7 - DNA, 1000 bp, Artificial Sequence
  Synthetic Sequence
gattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgttcccctgcatcccccttcccctgcatcccccagaggccccaggccacctacttggcctggaccccacgagaggccaccccagccctgtctaccaggctgccttttgggtggattctcctccaactgtggggtgactgcttgg

SEQ ID NO: 8 - DNA, 804 bp, Artificial Sequence
  Synthetic Sequence
tgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggc

SEQ ID NO: 9 - DNA, 23 bp, Artificial Sequence
  Synthetic Sequence
ccaatcctgtccctagtggcccc

SEQ ID NO: 10 - DNA, 39 bp, Artificial Sequence
  Synthetic Sequence
atcgatcgcaggcgcaatcttcgcatttcttttttccag

SEQ ID NO: 11 - DNA, 147 bp, Artificial Sequence
  Synthetic Sequence
cctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattc

SEQ ID NO: 12 - DNA, 708 bp, Artificial Sequence
  Synthetic Sequence
gtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 13 - DNA, 986 bp, Artificial Sequence
  Artificial Sequence
ttatctgtcccctccaccccacagtggggccactagggacagcgatcgggtacatcgatcgcaggcgcaatcttcgcatttcttttttccaggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacgcggccgccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcgattggtgacagaaaagccccatccttagg

SEQ ID NO: 14 - DNA, 1526 bp, Artificial Sequence
  Synthetic Sequence
gttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacagcgatcgggtacatcgatcgcaggcgcaatcttcgcatttcttttttccaggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacgcggccgccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcgattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtc

SEQ ID NO: 15 - DNA, 1926 bp, Artificial Sequence
  Synthetic Sequence
tcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacagcgatcgggtacatcgatcgcaggcgcaatcttcgcatttcttttttccaggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacgcggccgccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcgattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcag

SEQ ID NO: 16 - DNA, 2730 bp, Artificial Sequence
  Synthetic Sequence
tgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacagcgatcgggtacatcgatcgcaggcgcaatcttcgcatttcttttttccaggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacgcggccgccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcgattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgttcccctgcatcccccttcccctgcatcccccagaggccccaggccacctacttggcctggaccccacgagaggccaccccagccctgtctaccaggctgccttttgggtggattctcctccaactgtggggtgactgcttgg

SEQ ID NO: 17 - DNA, 20 bp, Artificial Sequence
  Synthetic Sequence
ggggccactagggacaggat

SEQ ID NO: 18 - DNA, 800 bp, Artificial Sequence
  Synthetic Sequence
atgctgctgctggtgacctctctgctgctgtgcgagctgccacacccagccttcctgctgatcccagacatccagatgacacagagccccagctccctgagcgcctccgtgggcgacagagtgaccatcacatgtagggcctctgagagcgtggataactatggcatcagcttcatgaattggtttcagcagaagcctggcggcgccccaaagctgctgatctacgcagccagcatgcagggctccggcgtgccctctcggttctccggctctggcagcggcaccgacttcaccctgacaatctctagcctgcagccagacgatttcgccacatactattgccagcagagcaaggaggtgccctggacctttggccagggcacaaaggtggagatcaagggctccacctctggcagcggcaagcctggcagcggagagggctccacaaagggacaggtgcagctggtgcagtccggagccgaggtgaagaagccaggctcctctgtgaaggtgtcttgtaaggccagcggctataccttcacagactacaacatgcactgggtgcgccaggcaccaggacagggcctggagtggatcggctacatctatccttacaacggcggcaccggctataatcagaagtttaagtccaaggccaccatcacagccgatgagtctaccaatacagcctacatggagctgagcagcctgcggtccgaggacacagccgtgtactattgcgcccggggcagacccgctatggactattggggccagggcaccctggtgacagtgtctag

SEQ ID NO: 19 - DNA, 687 bp, Artificial Sequence
  Synthetic Sequence
gagagcaagtacggaccaccttgcccaccatgtcctgcaccagagttcctgggaggaccttccgtgttcctgtttcctccaaagccaaaggacaccctgatgatcagccggaccccagaggtgacatgcgtggtggtggacgtgagccaggaggaccccgaggtgcagttcaactggtacgtggatggcgtggaggtgcacaatgccaagaccaagccaagagaggagcagtttaactccacctatagggtggtgtctgtgctgacagtgctgcaccaggactggctgaacggcaaggagtacaagtgcaaggtgtccaataagggcctgccttcctctatcgagaagaccatctctaaggcaaagggacagccaagggagccacaggtgtatacactgccccctagccaggaggagatgaccaagaaccaggtgtccctgacatgtctggtgaagggcttttacccttctgacatcgccgtggagtgggagagcaatggccagccagagaacaattataagaccacaccacccgtgctggactctgatggcagcttctttctgtacagccgcctgaccgtggataagtcccggtggcaggagggcaacgtgttctcctgctctgtgatgcacgaggccctgcacaatcactacacacagaagagcctgtccctgtctctgggcaag

SEQ ID NO: 20 - DNA, 207 bp, Artificial Sequence
  Synthetic Sequence
atgttttgggtgctggtggtggtgggaggcgtgctggcctgttattccctgctggtgaccgtggccttcatcatcttttgggtgcgctccaagcggagccggggcggacactctgactacatgaacatgaccccacggagacccggacctacaaggaagcactatcagccctacgcccctccacgggacttcgcagcatatcgcagc

SEQ ID NO: 21 - DNA, 339 bp, Artificial Sequence
  Synthetic Sequence
cgggtgaagtttagcagatccgccgatgcaccagcatatcagcagggacagaatcagctgtacaacgagctgaatctgggcaggcgcgaggagtacgacgtgctggataagaggcggggccgggaccccgagatgggaggcaagcccaggcgcaagaaccctcaggagggcctgtataatgagctgcagaaggacaagatggccgaggcctacagcgagatcggcatgaagggagagcggagaaggggcaagggacacgatggcctgtatcagggcctgtccaccgccacaaaggacacctacgatgcactgcacatgcaggccctgccacctcggtga

SEQ ID NO: 22 - DNA, 6864 bp, Artificial Sequence
  Synthetic Sequence
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccggcgcgccgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacagcgatcgggtacatcgatcacgagactagcctcgagaagcttgatatcgaattccacggggttggacgcgtcttaattaaggatccaaggtcaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatcgatcccccgggctgcaggaattcaagcgagaagacaagggcagaaagcaccgccaccatgctgctgctggtgacctctctgctgctgtgcgagctgccacacccagccttcctgctgatcccagacatccagatgacacagagccccagctccctgagcgcctccgtgggcgacagagtgaccatcacatgtagggcctctgagagcgtggataactatggcatcagcttcatgaattggtttcagcagaagcctggcggcgccccaaagctgctgatctacgcagccagcatgcagggctccggcgtgccctctcggttctccggctctggcagcggcaccgacttcaccctgacaatctctagcctgcagccagacgatttcgccacatactattgccagcagagcaaggaggtgccctggacctttggccagggcacaaaggtggagatcaagggctccacctctggcagcggcaagcctggcagcggagagggctccacaaagggacaggtgcagctggtgcagtccggagccgaggtgaagaagccaggctcctctgtgaaggtgtcttgtaaggccagcggctataccttcacagactacaacatgcactgggtgcgccaggcaccaggacagggcctggagtggatcggctacatctatccttacaacggcggcaccggctataatcagaagtttaagtccaaggccaccatcacagccgatgagtctaccaatacagcctacatggagctgagcagcctgcggtccgaggacacagccgtgtactattgcgcccggggcagacccgctatggactattggggccagggcaccctggtgacagtgtctagcgagagcaagtacggaccaccttgcccaccatgtcctgcaccagagttcctgggaggaccttccgtgttcctgtttcctccaaagccaaaggacaccctgatgatcagccggaccccagaggtgacatgcgtggtggtggacgtgagccaggaggaccccgaggtgcagttcaactggtacgtggatggcgtggaggtgcacaatgccaagaccaagccaagagaggagcagtttaactccacctatagggtggtgtctgtgctgacagtgctgcaccaggactggctgaacggcaaggagtacaagtgcaaggtgtccaataagggcctgccttcctctatcgagaagaccatctctaaggcaaagggacagccaagggagccacaggtgtatacactgccccctagccaggaggagatgaccaagaaccaggtgtccctgacatgtctggtgaagggcttttacccttctgacatcgccgtggagtgggagagcaatggccagccagagaacaattataagaccacaccacccgtgctggactctgatggcagcttctttctgtacagccgcctgaccgtggataagtcccggtggcaggagggcaacgtgttctcctgctctgtgatgcacgaggccctgcacaatcactacacacagaagagcctgtccctgtctctgggcaagatgttttgggtgctggtggtggtgggaggcgtgctggcctgttattccctgctggtgaccgtggccttcatcatcttttgggtgcgctccaagcggagccggggcggacactctgactacatgaacatgaccccacggagacccggacctacaaggaagcactatcagccctacgcccctccacgggacttcgcagcatatcgcagccgggtgaagtttagcagatccgccgatgcaccagcatatcagcagggacagaatcagctgtacaacgagctgaatctgggcaggcgcgaggagtacgacgtgctggataagaggcggggccgggaccccgagatgggaggcaagcccaggcgcaagaaccctcaggagggcctgtataatgagctgcagaaggacaagatggccgaggcctacagcgagatcggcatgaagggagagcggagaaggggcaagggacacgatggcctgtatcagggcctgtccaccgccacaaaggacacctacgatgcactgcacatgcaggccctgccacctcggtgaaagtaacgccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcgattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 23 - DNA, 7161 bp, Artificial Sequence
  Synthetic Sequence
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccggcgcgccgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacagcgatcgggtacatcgatcacgagactagcctcgagaagcttgatatcgaattccacggggttggacgcgtcttaattaaggatccaaggtcaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatcgatcccccgggctgcaggaattcaagcgagaagacaagggcagaaagcaccgccaccatgctgctgctggtgacctccctgctgctgtgcgagctgccacaccctgcctttctgctgatcccagacatccagatgacacagagccccagctccctgtctgccagcgtgggcgacagagtgaccatcacatgtagggcctccgagtctgtggataactatggcatcagctttatgaattggttccagcagaagccaggaggcgcccctaagctgctgatctacgcagcctccatgcagggctctggcgtgcccagccgctttagcggctccggctctggcaccgatttcaccctgacaatctctagcctgcagccagacgattttgccacatactattgccagcagtccaaggaggtgccctggaccttcggccagggcacaaaggtggagatcaagggcagcacctccggctctggcaagcctggctccggagagggctctacaaagggacaggtgcagctggtgcagagcggagccgaggtgaagaagccaggctcctctgtgaaggtgagctgtaaggcctccggctatacctttacagactacaacatgcactgggtgagacaggcaccaggacagggcctggagtggatcggctacatctatccttacaacggcggcaccggctataatcagaagttcaagagcaaggccaccatcacagccgatgagtccaccaatacagcctacatggagctgagcagcctgaggagcgaggacacagccgtgtactattgcgccagaggcaggcctgctatggactattggggccagggcaccctggtgacagtgtctagcgagtccaagtacggaccaccttgcccaccatgtccagcaccagagtttctgggaggacctagcgtgtttctgttccctccaaagccaaaggacaccctgatgatcagcagaacccccgaggtgacatgcgtggtggtggacgtgtcccaggaggaccccgaggtgcagtttaactggtacgtggatggcgtggaggtgcacaatgccaagaccaagcctagagaggagcagttcaactccacctatagggtggtgtctgtgctgacagtgctgcaccaggactggctgaacggcaaggagtacaagtgcaaggtgtctaataagggcctgccatcctctatcgagaagaccatcagcaaggccaagggccagcctagggagccacaggtgtatacactgcccccttcccaggaggagatgaccaagaaccaggtgtctctgacatgtctggtgaagggcttctacccatccgacatcgccgtggagtgggagtctaatggccagcccgagaacaattataagaccacaccacccgtgctggactctgatggcagcttctttctgtactctcgcctgaccgtggataagagccggtggcaggagggcaacgtgtttagctgctccgtgatgcacgaggccctgcacaatcactacacacagaagtctctgagcctgtccctgggcaagagcaacctgttcgtggcctcctggatcgccgtgatgatcatctttcgcatcggcatggccgtggccatcttctgctgtttctttttcccatccggaggctctggaggaggctccggctggcggagaaagcggaaggagaagcagagcgagacctcccctaaggagtttctgacaatctatgaggacgtgaaggatctgaagaccaggcgcaatcacgagcaggagcagaccttcccaggaggaggctctacaatctacagcatgatccagtcccagagcagcgccccaaccagccaggagccagcctatacactgtactctctgatccagcctagccggaagtctggcagccgcaagcggaaccactccccatctttcaattctaccatctatgaagtgatcggcaagagccagcctaaggcccagaacccagccagactgtccaggaaggagctggagaattttgacgtgtactctggaggcagcggaggaggctctggccgcgtgaagttcagccggtccgccgatgccccagcctataagcagggccagaaccagctgtacaacgagctgaatctgggccggagagaggagtacgacgtgctggataagaggcggggccgggaccccgagatgggaggcaagccccggagaaagaaccctcaggagggcctgtataatgagctgcagaaggacaagatggccgaggcctactccgagatcggcatgaagggagagaggcgccggggcaagggacacgatggcctgtatcagggcctgagcaccgccacaaaggacacctacgatgccctgcacatgcaggccctgcctccacggtgatgaaagtaacgccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcgattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 24 - DNA, 93 bp, Artificial Sequence
  Synthetic Sequence
agcaacctgttcgtggcctcctggatcgccgtgatgatcatctttcgcatcggcatggccgtggccatcttctgctgtttctttttcccatcc

SEQ ID NO: 25 - DNA, 24 bp, Artificial Sequence
  Synthetic Sequence
ggaggctctggaggaggctccggc

SEQ ID NO: 26 - DNA, 360 bp, Artificial Sequence
  Synthetic Sequence
tggcggagaaagcggaaggagaagcagagcgagacctcccctaaggagtttctgacaatctatgaggacgtgaaggatctgaagaccaggcgcaatcacgagcaggagcagaccttcccaggaggaggctctacaatctacagcatgatccagtcccagagcagcgccccaaccagccaggagccagcctatacactgtactctctgatccagcctagccggaagtctggcagccgcaagcggaaccactccccatctttcaattctaccatctatgaagtgatcggcaagagccagcctaaggcccagaacccagccagactgtccaggaaggagctggagaattttgacgtgtactct

SEQ ID NO: 27 - DNA, 24 bp, Artificial Sequence
  Synthetic Sequence
ggaggcagcggaggaggctctggc

SEQ ID NO: 28 - DNA, 342 bp, Artificial Sequence
  Synthetic Sequence
cgcgtgaagttcagccggtccgccgatgccccagcctataagcagggccagaaccagctgtacaacgagctgaatctgggccggagagaggagtacgacgtgctggataagaggcggggccgggaccccgagatgggaggcaagccccggagaaagaaccctcaggagggcctgtataatgagctgcagaaggacaagatggccgaggcctactccgagatcggcatgaagggagagaggcgccggggcaagggacacgatggcctgtatcagggcctgagcaccgccacaaaggacacctacgatgccctgcacatgcaggccctgcctccacggtgatga

SEQ ID NO: 29 - DNA, 801 bp, Artificial Sequence
  Synthetic Sequence
atgctgctgctggtgacctccctgctgctgtgcgagctgccacaccctgcctttctgctgatcccagacatccagatgacacagagccccagctccctgtctgccagcgtgggcgacagagtgaccatcacatgtagggcctccgagtctgtggataactatggcatcagctttatgaattggttccagcagaagccaggaggcgcccctaagctgctgatctacgcagcctccatgcagggctctggcgtgcccagccgctttagcggctccggctctggcaccgatttcaccctgacaatctctagcctgcagccagacgattttgccacatactattgccagcagtccaaggaggtgccctggaccttcggccagggcacaaaggtggagatcaagggcagcacctccggctctggcaagcctggctccggagagggctctacaaagggacaggtgcagctggtgcagagcggagccgaggtgaagaagccaggctcctctgtgaaggtgagctgtaaggcctccggctatacctttacagactacaacatgcactgggtgagacaggcaccaggacagggcctggagtggatcggctacatctatccttacaacggcggcaccggctataatcagaagttcaagagcaaggccaccatcacagccgatgagtccaccaatacagcctacatggagctgagcagcctgaggagcgaggacacagccgtgtactattgcgccagaggcaggcctgctatggactattggggccagggcaccctggtgacagtgtctagc

SEQ ID NO: 30 - DNA, 540 bp, Artificial Sequence
  Synthetic Sequence
atcgatcacgagactagcctcgagaagcttgatatcgaattccacggggttggacgcgtcttaattaaggatccaaggtcaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatcgatcccccgggctgcaggaattcaagcgagaagacaagggcagaaagcacc

SEQ ID NO: 31 - DNA, 604 bp, Artificial Sequence
  Synthetic Sequence
gctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggc

SEQ ID NO: 32 - DNA, 600 bp, Artificial Sequence
  Synthetic Sequence
gattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttct

SEQ ID NO: 33 - DNA, 20 bp, Artificial Sequence
  Synthetic Sequence
ggggccactagggacaggat

SEQ ID NO: 34 - DNA, 22 bp, Artificial Sequence
  Synthetic Sequence
ttctcctgtggattcgggtcac

SEQ ID NO: 35 - DNA, 22 bp, Artificial Sequence
  Synthetic Sequence
ctctctggctccatcgtaagca

SEQ ID NO: 36 - DNA, 19 bp, Artificial Sequence
  Synthetic Sequence
tcctgggcaaacagcataa

SEQ ID NO: 37 - DNA, 20 bp, Artificial Sequence
  Synthetic Sequence
gagctgcagaaggacaagat

SEQ ID NO: 38 - DNA, 22 bp, Artificial Sequence
  Synthetic Sequence
ctctgtgtcatctggatgtctg

SEQ ID NO: 39 - DNA, 22 bp, Artificial Sequence
  Synthetic Sequence
ctttgagctctactggcttctg

SEQ ID NO: 40 - DNA, 19 bp, Artificial Sequence
  Synthetic Sequence
tcctgggcaaacagcataa

SEQ ID NO: 41 - DNA, 22 bp, Artificial Sequence
  Synthetic Sequence
ctttgagctctactggcttctg

SEQ ID NO: 42 - DNA, 18 bp, Artificial Sequence
  Synthetic Sequence
gcgagtgaagacggcatg

SEQ ID NO: 43 - DNA, 20 bp, Artificial Sequence
  Synthetic Sequence
gtctgtgctagctcttccag

SEQ ID NO: 44 - DNA, 19 bp, Artificial Sequence
  Synthetic Sequence
gcgatgtcagaagggtaaa

SEQ ID NO: 45 - DNA, 20 bp, Artificial Sequence
  Synthetic Sequence
ggcggacactctgactacat

SEQ ID NO: 46 - DNA, 235 bp, Artificial Sequence
  Synthetic Sequence
ggcatggggttgggtgagggaggagagatgcccggagaggacccagacacggggaggatccgctcagaggacatcacgtggtgcagcggcgcgccggccgcagaaagggagtagaggcggccacgacctggtgaacacctaggacgcaccattctcacaaagggagttttccacacggacacccccctcctcaccacagccctgccaggacggggctggctactggccttatctc

SEQ ID NO: 47 - DNA, 243 bp, Artificial Sequence
  Synthetic Sequence
gcgagtgaagacggcatggggttgggtgagggaggagagatgcccggagaggacccagacacggggaggatccgctcagaggacatcacgtggtgcagcggcgcgccggccgcaggaagggagtagaggcggccacgacctggtgaacacctaggacgcaccattctcacaaagggagttttccacacggacacccccctcctcaccacagccctgccaggacggggctggctactggcctta

SEQ ID NO: 48 - DNA, 229 bp, Artificial Sequence
  Synthetic Sequence
gcgagtgaagacggcatggggttgggtgagggaggagagatgcccggagaggacccagacacggggaggatccgctcagaggacatcacgtggtgcagcggcgcgcagagagggagtggccaactccatcactaggggttcctgcggccgcagaaagggagtagaggcggccacgacctggtgaacacctaggacgcaccattctcacaaagggagttttccacacgga

SEQ ID NO: 49 - DNA, 234 bp, Artificial Sequence
  Synthetic Sequence
gcgagtgaagacggcatggggttgggtgagggaggagagatgcccggagaggacccagacacggggaggatccgctcagaggacatcacgtggtgcagcggccgcagaaagggagtagaggcggccacgacctggtgaacacctaggacgcaccattctcacaaagggagttttccacacggacacccccctcctcaccacagccctgccaggacggggctggctactggcctt

SEQ ID NO: 50 - DNA, 972 bp, Artificial Sequence
  Synthetic Sequence
ccaatcctgtccctagtggcccccactagggacagcgatcgggtacatcgatcgcaggcgcaatcttcgcatttcttttttccaggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacgcggccgccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcccaatcctgtccctagtggcccc

SEQ ID NO: 51 - DNA, 949 bp, Artificial Sequence
  Synthetic Sequence
ccaatcctgtccctagtggcccccactagggacagcgatcgggtacatcgatcgcaggcgcaatcttcgcatttcttttttccaggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacgcggccgccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattc

SEQ ID NO: 52 - DNA, 65 bp, Artificial Sequence
  Synthetic Sequence
cccctccaccccacagtggggccactagggacaggattggtgacagaaaagccccatccttaggc

SEQ ID NO: 53 - DNA, 34 bp, Artificial Sequence
  Synthetic Sequence
cccctccaccccacagtggggccactagggacag

SEQ ID NO: 54 - DNA, 30 bp, Artificial Sequence
  Synthetic Sequence
attggtgacagaaaagccccatccttaggc

SEQ ID NO: 55 - DNA, 31 bp, Artificial Sequence
  Synthetic Sequence
cccctccaccccacagtggggccactaggga

SEQ ID NO: 56 - DNA, 14 bp, Artificial Sequence
  Synthetic Sequence
cccctccaccccac

SEQ ID NO: 57 - DNA, 25 bp, Artificial Sequence
  Synthetic Sequence
cccctccaccccacagtggggccac

SEQ ID NO: 58 - DNA, 31 bp, Artificial Sequence
  Synthetic Sequence
gattggtgacagaaaagccccatccttaggc